(19) United States
(12) Patent Application Publication
(10) Pub. No.: US 2014/0356962 A1
(43) Pub. Date: Dec. 4, 2014

(54) NOVEL ATTENUATED POLIOVIRUS: PV-1 MONO-CRE-X

(71) Applicant: THE RESEARCH FOUNDATION FOR THE STATE UNIVERSITY OF NEW YORK, Albany, NY (US)

(72) Inventors: Eckard Wimmer, East Setauket, NY (US); Jeronimo Cello, Port Jefferson, NY (US); Ying Liu, Port Jefferson, NY (US)

(21) Appl. No.: 14/365,854

(22) PCT Filed: Dec. 14, 2012
(86) PCT No.: PCT/US12/69868

§ 371 Date: Jun. 16, 2014

Related U.S. Application Data
(60) Provisional application No. 61/576,706, filed on Dec. 16, 2011.

(51) Int. Cl. C12N 7/00

(52) U.S. Cl. CPC C12N 7/00; C12N 2770/32321; C12N 2770/32361; C12N 2770/32334
USPC 435/471; 435/236; 536/23.2

(57) ABSTRACT

A novel and stable attenuated poliovirus is produced by engineering an indigenous replication element (cre), into the 5′ non-translated genomic region (with inactivation of the native ere element located in the coding region of 2C (mono-crePV), and replacing the nucleic acid sequence of all or part of the capsid coding region (PI) with a substitute PI coding region having reduced codon pair bias. The stably attenuated poliovirus is effective for vaccines and immunization.

### FIELD OF THE INVENTION

The present invention relates to novel attenuated polioviruses. The attenuated polioviruses exhibit excellent growth phenotypes in tissue culture while stably maintaining extremely low neurovirulence. Accordingly, the attenuated viruses are preferred for use in vaccines.

### CROSS-REFERENCE TO RELATED APPLICATIONS

This application claims priority to U.S. Application No. 61/576,706, filed Dec. 16, 2011, which is incorporated herein by reference in its entirety.

### BACKGROUND OF THE INVENTION

The poliovirus (“PV”) genome contains a hairpin structure, called the cis acting replication element (cre), which is essential for genome replication. The cre element is located in the coding sequence of the 2C ATPase protein (within the P2 domain). Specific mutations in this loop lead to a non-replicating phenotype. A PV genome with an inactivated cre in P2 can be revived by inserting a synthetic cre elsewhere into the PV genome, such as the 5′ non-translated region (5′NTR). Insertion of the synthetic cre between the cloverleaf and the IRES of the 5′NTR, yielded a variant, termed PV-1(mono-cre), that replicated in HeLa cells with wild type kinetics. In transgenic mice that express CD155, the poliovirus receptor, PV-1(mono-cre) is attenuated, having a LD₅₀ of greater than 10⁷ particles. Nevertheless, PV-1(mono-cre) grows well in Vero and MRC5 cells at 33° C. and 37° C. The virus is resistant to loss of attenuation as the cre element must be maintained for viral replication.

The frequencies with which codons occur next to one another in the open reading frames of human mRNAs are not what is statistically expected from the frequency with which each of the codons alone are used. There are codon pairs in each gene of the human genome that are overrepresented and codon pairs that are underrepresented. Utilizing whole genome chemical synthesis we have shown that an excess of underrepresented codon pairs attenuates expression of a viral gene. Furthermore, codon pair deoptimization (increasing underrepresented codon pairs relative to over represented codon pairs) in one or several ORFs of a virus can lead to profound attenuation.

We have previously constructed a codon pair deoptimized poliovirus genome, PV-X. In that PV genome, the 759 nt “X” segment (the first third of the capsid domain) contains 181 nucleotide changes as compared to the wt PV genome. This design of the PV genome is not only attenuated, but has the advantage that the attenuated phenotype will not revert to full virulence because of the large number of mutations in X. In the context of the entire segment, a single nucleotide reversion confers only a very small difference, if any, in phenotype.

### SUMMARY OF THE INVENTION

The invention provides attenuated polioviruses that exhibit excellent growth phenotypes in tissue culture while stably maintaining extremely low neurovirulence. The invention provides an attenuated poliovirus genome comprising a single active cis acting replication element (cre) located in the spacer region of the 5′-NTR between the cloverleaf and internal ribosome entry site (IRES), and a poliovirus protein encoding sequence having a codon pair bias less than the codon pair bias of the parent poliovirus protein encoding sequence from which it is derived. In an embodiment of the invention, the active cre element is inserted at nucleotide 102/103. In an embodiment of the invention, the cre element is positioned as in SEQ ID NO:1.

In an embodiment of the invention, the protein encoding sequence has a codon pair bias at least about 0.05 less, or at least about 0.1 less, at least about 0.2 less than the codon pair bias of the parent protein encoding sequence. In an embodiment of the invention, the protein encoding sequence has a codon pair bias of about −0.05 or less, or about −0.1 or less, or about −0.3 or less, or about −0.4 or less. In an embodiment of the invention, the protein encoding sequence is less than 90% identical or less than 80% identical to the protein encoding sequence of the parent virus.

In an embodiment of the invention, while codons are exchanged within the sequence, the resulting protein encoding sequence and the parent protein encoding sequence encode the same protein. In an embodiment of the invention, the protein sequence is that of a natural isolate of the protein. In an embodiment of the invention, the encoded protein is of strain Mahoney.

In certain embodiments, the attenuated poliovirus genome encodes a protein that differs from a natural isolate by about 10 amino acids or fewer or by about 20 amino acids or fewer. In certain embodiments, the attenuated poliovirus genome encodes a protein that differs from the parental protein from which it is derived by about 10 amino acids or fewer or by about 20 amino acids or fewer. In an embodiment of the invention, the attenuated poliovirus genome comprises the nucleotide sequence of PV-Min (SEQ ID NO:2) from nucleotide 755 to nucleotide 1514. In another embodiment, the attenuated poliovirus genome comprises SEQ ID NO:3.

The invention provides an attenuated poliovirus comprising any one of the abovementioned poliovirus genomes. The invention further provides vaccine compositions comprising such attenuated polioviruses, as well as a method for eliciting an immune response in a subject by administering to the subject an effective dose of the vaccine compositions.

The invention provides a method of making an attenuated poliovirus genome, which comprises preparing a nucleic acid sequence which comprises a cis acting replication element (cre) located in the spacer region of the 5′-NTR between the cloverleaf and internal ribosome entry site (IRES), and a poliovirus protein encoding nucleotide sequence having a codon pair bias less than the codon pair bias of the parent poliovirus protein encoding sequence from which it is derived. According to the invention, the reduced codon-pair bias protein encoding sequence is made by rearranging the codons of the parent poliovirus nucleotide sequence. In an embodiment of the invention, the rearranged sequence encodes the identical protein as the parent poliovirus nucleotide sequence. The invention further provides for introducing the poliovirus into an appropriate host cell and culturing the host cell to produce the poliovirus. The invention also provides a kit comprising such recombinant polioviruses and instructional material for their use.

### DESCRIPTION OF THE FIGURES

FIG. 1. Genome structure of wild type PV, and the parental attenuated PV strains. The genomic structures of poliovirus type 1 Mahoney (“PV(M)”) and chimeric viruses used are illustrated. IRES sequences of PVM are shown in black. The cre element in the 2C coding region is indicated as an stem-loop. In the mono-cre strain, the native cre(2C) was inactivated through mutations (Toyoda, H., et al., Cancer Res Mar. 15, 2007, 67; 2857) in its conserved sequence as indicated by “X”. A second copy of wild type cre was inserted between the cloverleaf and the IRES. In the strain designated PV-X, part of the P1 coding region (nt 755-nt 1513, indicated as gray bar) contains under-represented codon pairs (Coleman, J. R., et al., Science, Jun. 27, 2008, 320(5884):1784-7).

FIG. 2. Growth phenotypes of PVM and PV-1(mono cre-X). The genomic structures of PVM and PV-1(mono cre-X) are illustrated on the left. RNA transcripts were transfected into HeLa (R19) cells and the viruses obtained at CPE, if any, were titered by a plaque assay.

FIG. 3. One step growth curve for Mono-cre-X virus in HeLa (R19), MRC5 and 293T cells. Cells were infected at a MOI of 0.5 and incubated at 33° C., 37° C. and 39.5° C. The virus titer was determined by plaque assay on monolayers of HeLa (R19) cells.

FIG. 4. Genomic organization of PV(M) poliovirus, mono-crePV and dual-crePV. The single-stranded RNA is covalently linked to the viral-encoded protein VPg at the 5′ end of the non-translated region (5′-NTR). The 5′-NTR consists of two cis-acting domains, the cloverleaf and the internal ribosomal entry site (IRES), which are separated by a spacer region. The IRES controls translation of the polyprotein (open box), consisting of structural (P1) region and nonstructural regions (P2 and P3), specifying the replication proteins. Within the 2C^{ATPase} coding region, the cis replication element (cre) is indicated. The 3′-NTR contains a heteropolymeric region and is polyadenylylated. RNA replication requires all three structural elements, cloverleaf, cre and the 3′-NTR. The duplicated cre was inserted into the spacer between cloverleaf and IRES (dual-crePV). The native cre in 2C^{ATPase} was inactivated by mutation as indicated by an X at the stem loop (mono-crePV).

FIG. 5. (A) Determining codon pair bias of human and viral ORFs. Each dot represents the average codon-pair score per codon pair for one ORF plotted against its length. Codon pair bias (CPB) was calculated for 14,795 annotated human genes. Under-represented codon pairs yield negative scores. CPB is plotted for various poliovirus P1 constructs, represented by symbols with arrows. The figure illustrates that the bulk of human genes clusters around 0.1. CPB is shown for PV(M)-wt (labeled “WT”) (−0.02), customized synthetic poliovirus capsids PV-Max (+0.25), PV-Min (−0.48), and PV(M)-wt:PV-Min chimera capsids PV-Min⁷⁵⁵⁻²⁴⁷⁰ (=“PV-MinXY”) (−0.31) and PV-Min²⁴⁷⁰⁻³³⁸⁶ (=“PV-MinZ”) (−0.20). (B) The structures of the various chimeric, partly synthetic poliovirus constructs are depicted. PV-Min X is also referred to herein as PV-X.

### DETAILED DESCRIPTION

The invention provides highly attenuated polioviruses that are suitable for vaccines and for the treatment or amelioration of human solid tumors, such as neuroblastoma in children. Attenuated polioviruses of the invention comprise two features that modulate attenuation. One feature is insertion of the hairpin structure known as the cis acting replication element (cre) into the 5′ nontranslated (5′NTR) of the poliovirus genome. The second is reduction of codon pair bias in protein encoding portions of the poliovirus genome.

According to the invention, poliovirus isolates can be stably attenuated, and replicative properties enhanced. Such poliovirus can be naturally occurring isolates, or derivatives thereof. Poliovirus type 1 (Mahoney) (PV 1(M)) is exemplified herein. Other non-limiting examples of neurovirulent poliovirus include P3/Leon/37 (from which the attenuated Sabin vaccine is derived) and neurovirulent derivatives of those P3/Leon/37 and Mahoney. For example, non-attenuating mutations present in attenuated poliovirus (such as Sabin) have been distinguished in the art from those that cause attenuation. Further examples are poliovirus isolates from individuals who chronically excrete poliovirus of vaccine-origin.

Cre Insertion into 5′ NTR

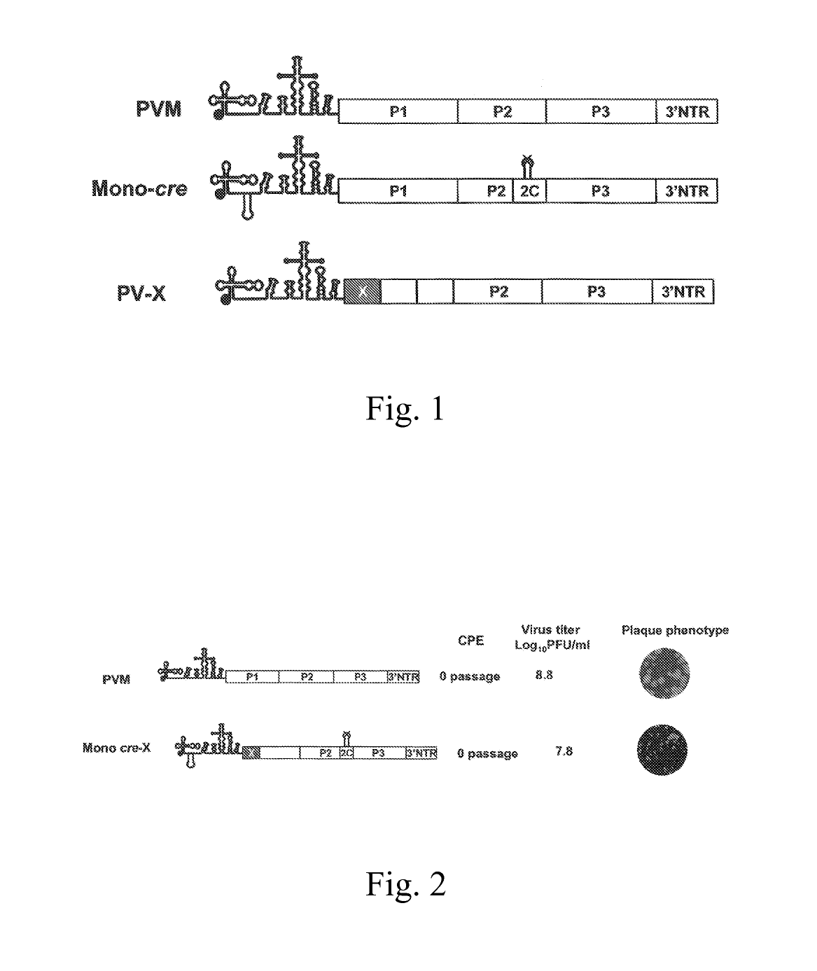

A stable attenuated phenotype is generated when the spacer region between cloverleaf and IRES of the poliovirus genome is interrupted by an essential RNA replication element that the virus cannot afford to delete. Such an element is the cre, a stem-loop structure mapping to the coding region of viral protein 2C^{ATPase} in native poliovirus (FIG. 1, top). According to the invention, a single active cre element is provided in the 5′-NTR of the poliovirus genome at a position which results in viral attenuation, and wherein any mutation of the element that would revert the attenuation results in inactivation of the cre element such that the poliovirus becomes non-viable. According to the invention, an active cre element is inserted into the spacer region of the 5′-NTR between the cloverleaf and the internal ribosome entry site (IRES). In a particular embodiment, the cre element is inserted into the spacer region at nucleotides 102/103. Such reengineering of a cre element is described in U.S. Pat. No. 8,066,983, which is incorporated herein by reference.

It will be appreciated that the stability of attenuation depends on the cre element located in the 5′-NTR being the only active cre element. Accordingly, the native cre element, located in the 2C coding region of the poliovirus genome, is inactivated. Typically, the sequence of the native cre element, which is in a coding region, is mutated to inactivate the cre element, but not alter the amino acids encoded by the nucleotides of the cre element. However, mutations that result in conservative amino acid substitutions are allowable. A conservative amino acid substitution is a substitution with an amino acid having generally similar properties (e.g., acidic, basic, aromatic, size, positively or negatively charged, polarity, non-polarity) such that the substitutions do not substantially alter peptide, polypeptide or protein characteristics (e.g., charge, isoelectric point, affinity, avidity, conformation, and solubility) or activity. Typical substitutions that may be performed for such conservative amino acid substitution may be among the groups of amino acids as follows:

glycine (G), alanine (A), valine (V), leucine (L) and isoleucine (I);

aspartic acid (D) and glutamic acid (E);

alanine (A), serine (S) and threonine (T);

histidine (H), lysine (K) and arginine (R):

asparagine (N) and glutamine (Q);

phenylalanine (F), tyrosine (Y) and tryptophan (W).

According to the invention, a cre element is inserted into the 5′-NTR between the cloverleaf and the internal ribosome entry site (IRES) such that an attenuated virus results. As exemplified herein, a cre element is inserted into an NheI site created at nucleotide 102/103 in the 5′-NTR of PV1(M) (see SEQ ID NO:1), but need not be so precisely located. Attenuation may be determined, for example, by plaque assay or other techniques that are known in the art for measuring virus replication. cre element have been identified in the genomes of several picornaviruses, including poliovirus types 1 and 3, human rhinovirus (e.g., HRV2 and HRV14), cardioviruses. The cre elements are predicted to form hairpin structures with a conserved sequence of about 14 nucleotides at the loop portion of the hairpin. In an embodiment of the invention, the cre element is from the poliovirus type 1 designated PV1(M).

As exemplified herein, the replicative properties of an attenuated poliovirus can be enhanced by passage, in vitro, and in vivo. As demonstrated herein, mutations occur in attenuated viruses of the invention during passage, but are not observed to occur in the cre element engineered into the 5′-NTR. Accordingly, viral attenuation is not overcome. Rather, the mutations provide for enhancement of replication properties that are beneficial for oncolytic treatment of tumors. Further, such mutations are readily obtainable. Accordingly, the invention provides a stably attenuated poliovirus containing a single active cre regulatory element in the 5′-NTR.

Codon Pair Bias

Most amino acids are encoded by more than one codon. For instance, alanine is encoded by GCU, GCC, GCA, and GCG. Three amino acids (Leu, Ser, and Arg) are encoded by six different codons, while only Trp and Met have unique codons. “Synonymous” codons are codons that encode the same amino acid. Thus, for example, CUU, CUC, CUA, CUG, UUA, and UUG are synonymous codons that code for Leu. Synonymous codons are not used with equal frequency. In general, the most frequently used codons in a particular organism are those for which the cognate tRNA is abundant, and the use of these codons enhances the rate and/or accuracy of protein translation. Conversely, tRNAs for the rarely used codons are found at relatively low levels, and the use of rare codons is thought to reduce translation rate and/or accuracy. Thus, to replace a given codon in a nucleic acid by a synonymous but less frequently used codon is to substitute a “deoptimized” codon into the nucleic acid.

TABLE 1
Codon usage in Homo sapiens (source: http://www.
kazusa.or.jp/codon/)

| Amino Acid | Codon | Number | /1000 | Fraction |
|---|---|---|---|---|
| Gly | GGG | 636457.00 | 16.45 | 0.25 |
| Gly | GGA | 637120.00 | 16.47 | 0.25 |
| Gly | GGT | 416131.00 | 10.76 | 0.16 |
| Gly | GGC | 862557.00 | 22.29 | 0.34 |
| Glu | GAG | 1532589.00 | 39.61 | 0.58 |
| Glu | GAA | 1116000.00 | 28.84 | 0.42 |
| Asp | GAT | 842504.00 | 21.78 | 0.46 |
| Asp | GAC | 973377.00 | 25.16 | 0.54 |
| Val | GTG | 1091853.00 | 28.22 | 0.46 |
| Val | GTA | 273515.00 | 7.07 | 0.12 |
| Val | GTT | 426252.00 | 11.02 | 0.18 |
| Val | GTC | 562086.00 | 14.53 | 0.24 |
| Ala | GCG | 286975.00 | 7.42 | 0.11 |
| Ala | GCA | 614754.00 | 15.89 | 0.23 |
| Ala | GCT | 715079.00 | 18.48 | 0.27 |
| Ala | GCC | 1079491.00 | 27.90 | 0.40 |
| Arg | AGG | 461676.00 | 11.93 | 0.21 |
| Arg | AGA | 466435.00 | 12.06 | 0.21 |
| Ser | AGT | 469641.00 | 12.14 | 0.15 |
| Ser | AGC | 753597.00 | 19.48 | 0.24 |
| Lys | AAG | 1236148.00 | 31.95 | 0.57 |
| Lys | AAA | 940312.00 | 24.30 | 0.43 |
| Asn | AAT | 653566.00 | 16.89 | 0.47 |
| Asn | AAC | 739007.00 | 19.10 | 0.53 |
| Met | ATG | 853648.00 | 22.06 | 1.00 |
| Ile | ATA | 288118.00 | 7.45 | 0.17 |
| Ile | ATT | 615699.00 | 15.91 | 0.36 |
| Ile | ATC | 808306.00 | 20.89 | 0.47 |
| Thr | ACG | 234532.00 | 6.06 | 0.11 |
| Thr | ACA | 580580.00 | 15.01 | 0.28 |
| Thr | ACT | 506277.00 | 13.09 | 0.25 |
| Thr | ACC | 732313.00 | 18.93 | 0.36 |
| Trp | TGG | 510256.00 | 13.19 | 1.00 |
| End | TGA | 59528.00 | 1.54 | 0.47 |
| Cys | TGT | 407020.00 | 10.52 | 0.45 |
| Cys | TGC | 487907.00 | 12.61 | 0.55 |
| End | TAG | 30104.00 | 0.78 | 0.24 |
| End | TAA | 38222.00 | 0.99 | 0.30 |
| Tyr | TAT | 470083.00 | 12.15 | 0.44 |
| Tyr | TAC | 592163.00 | 15.30 | 0.56 |
| Leu | TTG | 498920.00 | 12.89 | 0.13 |
| Leu | TTA | 294684.00 | 7.62 | 0.08 |
| Phe | TTT | 676381.00 | 17.48 | 0.46 |
| Phe | TTC | 789374.00 | 20.40 | 0.54 |
| Ser | TCG | 171428.00 | 4.43 | 0.05 |
| Ser | TCA | 471469.00 | 12.19 | 0.15 |
| Ser | TCT | 585967.00 | 15.14 | 0.19 |
| Ser | TCC | 684663.00 | 17.70 | 0.22 |
| Arg | CGG | 443753.00 | 11.47 | 0.20 |
| Arg | CGA | 239573.00 | 6.19 | 0.11 |
| Arg | CGT | 176691.00 | 4.57 | 0.08 |
| Arg | CGC | 405748.00 | 10.49 | 0.18 |
| Gln | CAG | 1323614.00 | 34.21 | 0.74 |
| Gln | CAA | 473648.00 | 12.24 | 0.26 |
| His | CAT | 419726.00 | 10.85 | 0.42 |
| His | CAC | 583620.00 | 15.08 | 0.58 |
| Leu | CTG | 1539118.00 | 39.78 | 0.40 |
| Leu | CTA | 276799.00 | 7.15 | 0.07 |
| Leu | CTT | 508151.00 | 13.13 | 0.13 |
| Leu | CTC | 759527.00 | 19.63 | 0.20 |
| Pro | CCG | 268884.00 | 6.95 | 0.11 |
| Pro | CCA | 653281.00 | 16.88 | 0.28 |
| Pro | CCT | 676401.00 | 17.48 | 0.29 |
| Pro | CCC | 767793.00 | 19.84 | 0.32 |

In addition, a given organism has a preference for the nearest codon neighbor of a given codon, referred to as bias in codon pair utilization. A change of codon pair bias can influence the rate of protein synthesis and production of a protein. Importantly, a gene can be designed with underrepresented codon pairs that does not make use of different codons (no change in codon bias) and/or does not change the amino acid sequence of the encoded protein. Thus, replacement of a given codon pair by a less frequently used codon pair that encodes the same amino acids is to deoptimize codon pair usage.

Codon pair bias may be illustrated by considering the amino acid pair Ala-Glu, which can be encoded by 8 different codon pairs. If no factors other than the frequency of each individual codon are responsible for the frequency of the codon pair, the expected frequency of each of the 8 encodings can be calculated by multiplying the frequencies of the two relevant codons. For example, by this calculation the codon pair GCA-GAA would be expected to occur at a frequency of 0.097 out of all Ala-Glu coding pairs (0.23×0.42; based on the frequencies in Table 1). In order to relate the expected (hypothetical) frequency of each codon pair to the actually observed frequency in the human genome the Consensus CDS (CCDS) database of consistently annotated human coding regions, containing a total of 14,795 human genes, was used. This set of genes is the most comprehensive representation of human coding sequences. Using this set of genes the frequencies of codon usage were re-calculated by dividing the number of occurrences of a codon by the number of all synonymous codons coding for the same amino acid. As expected the frequencies correlated closely with previously published ones such as the ones given in Table 1. Slight frequency variations are possibly due to an oversampling effect in the data provided by the codon usage database at Kazusa DNA Research Institute (http://www.kazusa.or.jp/codon/codon.html) where 84949 human coding sequences were included in the calculation (far more than the actual number of human genes). The codon frequencies thus calculated were then used to calculate the expected codon-pair frequencies by first multiplying the frequencies of the two relevant codons with each other (see Table 2 expected frequency), and then multiplying this result with the observed frequency (in the entire CCDS data set) with which the amino acid pair encoded by the codon pair in question occurs. In the example of codon pair GCA-GAA, this second calculation gives an expected frequency of 0.098 (compared to 0.97 in the first calculation using the Kazusa dataset). Finally, the actual codon pair frequencies as observed in a set of 14,795 human genes was determined by counting the total number of occurrences of each codon pair in the set and dividing it by the number of all synonymous coding pairs in the set coding for the same amino acid pair (Table 3; observed frequency). Frequency and observed/expected values for the complete set of 3721 (61²) codon pairs, based on the set of 14,795 human genes, are provided herewith as Supplemental Table 1.

TABLE 2
Codon Pair Scores Exemplified by the Amino Acid
Pair Ala-Glu

| amino acid | codon | expected | observed | obs/exp |
|---|---|---|---|---|
| pair | pair | frequency | frequency | ratio |
| AE | GCAGAA | 0.098 | 0.163 | 1.65 |
| AE | GCAGAG | 0.132 | 0.198 | 1.51 |
| AE | GCCGAA | 0.171 | 0.031 | 0.18 |
| AE | GCCGAG | 0.229 | 0.142 | 0.62 |
| AE | GCGGAA | 0.046 | 0.027 | 0.57 |
| AE | GCGGAG | 0.062 | 0.089 | 1.44 |
| AE | GCTGAA | 0.112 | 0.145 | 1.29 |
| AE | GCTGAG | 0.150 | 0.206 | 1.37 |
| Total | | 1.000 | 1.000 | |

If the ratio of observed frequency/expected frequency of the codon pair is greater than one the codon pair is said to be overrepresented. If the ratio is smaller than one, it is said to be underrepresented. In the example the codon pair GCA-GAA is overrepresented 1.65 fold while the coding pair GCC-GAA is more than 5-fold underrepresented.

Many other codon pairs show very strong bias; some pairs are under-represented, while other pairs are over-represented. For instance, the codon pairs GCCGAA (AlaGlu) and GATCTG (AspLeu) are three- to six-fold under-represented (the preferred pairs being GCAGAG and GACCTG, respectively), while the codon pairs GCCAAG (AlaLys) and AATGAA (AsnGlu) are about two-fold over-represented. It is noteworthy that codon pair bias has nothing to do with the frequency of pairs of amino acids, nor with the frequency of individual codons. For instance, the under-represented pair GATCTG (AspLeu) happens to use the most frequent Leu codon, (CTG).

As discussed more fully below, codon pair bias takes into account the score for each codon pair in a coding sequence averaged over the entire length of the coding sequence. According to the invention, codon pair bias is determined by

$$CPB = {\sum }_{i = 1}^{k} \frac{CPSi}{k - 1} .$$

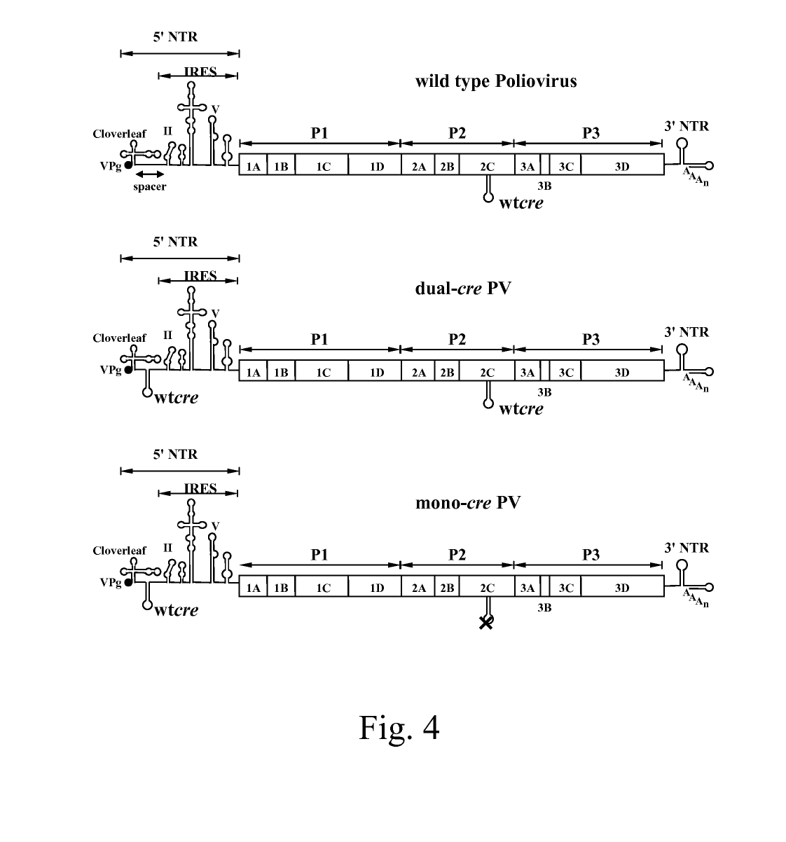

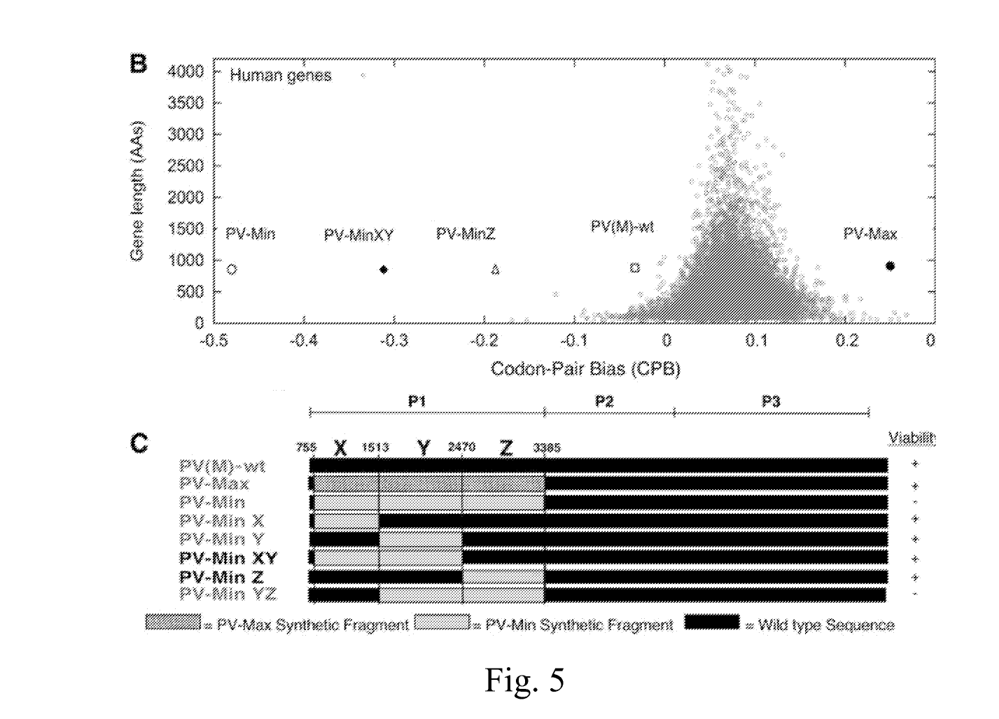

Accordingly, similar codon pair bias for a coding sequence can be obtained, for example, by minimized codon pair scores over a subsequence or moderately diminished codon pair scores over the full length of the coding sequence. For example, FIG. 4A shows intermediate codon pair reductions for P1 sequences that contain the “XY” or “Z” fragment of PV-Min. The same codon-pair bias reduction reached by PV-MinZ could be accomplished by exchanging codons within the whole P1 sequence, but with less extreme reductions per codon pair. Attenuation of poliovirus by reduction of codon pair bias is disclosed by PCT/US/2008/058952, which is incorporated herein by reference.

Calculation of Codon Pair Bias.

Every individual codon pair of the possible non-“STOP” containing codon pairs (e.g., GTT-GCT) carries an assigned “codon pair score,” or “CPS” that is specific for a given “training set” of genes. The CPS of a given codon pair is defined as the log ratio of the observed number of occurrences over the number that would have been expected in this set of genes (in this example the human genome). Determining the actual number of occurrences of a particular codon pair (or in other words the likelihood of a particular amino acid pair being encoded by a particular codon pair) is simply a matter of counting the actual number of occurrences of a codon pair in a particular set of coding sequences. Determining the expected number, however, requires additional calculations. The expected number is calculated so as to be independent of both amino acid frequency and codon bias similarly to Gutman and Hatfield. That is, the expected frequency is calculated based on the relative proportion of the number of times an amino acid is encoded by a specific codon. A positive CPS value signifies that the given codon pair is statistically over-represented, and a negative CPS indicates the pair is statistically under-represented in a genome.

To perform these calculations within the human context, the Consensus CDS (CCDS) database of annotated human coding regions, containing a total of 14,795 genes, was used. This data set provided codon and codon pair, and thus amino acid and amino-acid pair frequencies on a genomic scale.

The paradigm of Federov et al. (2002), was used to further enhanced the approach of Gutman and Hatfield (1989). This allowed calculation of the expected frequency of a given codon pair independent of codon frequency and non-random associations of neighboring codons encoding a particular amino acid pair.

$$S ( {P}_{ij} ) = ln ( \frac{{N}_{O} ( {P}_{ij} )}{{N}_{E} ( {P}_{ij} )} ) = ln ( \frac{{N}_{O} ( {P}_{ij} )}{F ( {C}_{i} ) F ( {C}_{j} ) {N}_{O} ( {X}_{ij} )} )$$

In the calculation, Pᵢⱼ is a codon pair occurring with a frequency of N_{O}(Pᵢⱼ) in its synonymous group. Cᵢ and Cⱼ are the two codons comprising Pᵢⱼ, occuring with frequencies F(Cᵢ) and F(Cⱼ) in their synonymous groups respectively. More explicitly, F(Cᵢ) is the frequency that corresponding amino acid Xᵢ is coded by codon Cᵢ throughout all coding regions and F(Cᵢ)=N_{O}(Cᵢ)/N_{O}(Xᵢ), where N_{O}(Cᵢ) and N_{O}(Xᵢ) are the observed number of occurrences of codon Cᵢ and amino acid Xᵢ respectively. F(Cⱼ) is calculated accordingly. Further, N_{O}(Xᵢⱼ) is the number of occurrences of amino acid pair Xᵢⱼ throughout all coding regions. The codon pair bias score S(Pᵢⱼ) of Pᵢⱼ was calculated as the log-odds ratio of the observed frequency Nₒ(Pᵢⱼ) over the expected number of occurrences of Nₑ(Pᵢⱼ).

Using the formula above, it was then determined whether individual codon pairs in individual coding sequences are over- or under-represented when compared to the corresponding genomic Nₑ(Pᵢⱼ) values that were calculated by using the entire human CCDS data set. This calculation resulted in positive S(Pᵢⱼ) score values for over-represented and negative values for under-represented codon pairs in the human coding regions (FIG. 4).

The “combined” codon pair bias of an individual coding sequence was calculated by averaging all codon pair scores according to the following formula:

$$S ( {P}_{ij} ) = {\sum }_{l = 1}^{k} \frac{S ( Pij ) l}{k - 1} .$$

The codon pair bias of an entire coding region is thus calculated by adding all of the individual codon pair scores comprising the region and dividing this sum by the length of the coding sequence.

The attenuated PV may be derived from poliovirus type 1 (Mahoney; “PV(M)”), poliovirus type 2 (Lansing), poliovirus type 3 (Leon), monovalent oral poliovirus vaccine (OPV) virus, or trivalent OPV virus. In certain embodiments, the attenuated poliovirus comprises all or part of the capsid coding region (the P1 region from nucleotide 755 to nucleotide 3385) of PV-Min (SEQ ID NO:1), which was redesigned from PV(M) to introduce the largest possible number of rarely used codon pairs. In certain embodiments, the attenuated poliovirus comprises nucleotides of PV-Min from nucleotides 755-1513 (e.g., PV-Min X), from nucleotides 755-2470 (e.g., PV-Min XY), from nucleotides 1513-3385 (e.g., PV-Min YZ), from nucleotides 2470-3385 (e.g., PV-Min Z), or from nucleotides 1513-2470 (e.g., PV-Min Y). The nomenclature reflects a poliovirus genome in which portions of the PV coding region are substituted with nucleotides of PV-Min. The invention is not limited to the abovementioned portions.

It will be understood that many nucleotide sequences can be generated by shuffling synonymous codons within a coding sequence. For example, starting from any particular protein encoding nucleotide sequence, a large number of nucleotide sequences can be generated that encode the same protein sequence and have reduced codon pair bias by shuffling the synonymous codons existing in that sequence. Accordingly, attenuated viruses of the invention include those which comprise nucleotide sequences specifically exemplified herein, as well as other nucleotide sequences with reduced codon pair bias that encode the same PV proteins.

The invention also encompasses variation in poliovirus protein sequences, including amino acid sequence variation among poliovirus isolates, as well as amino acid changes that may be introduced, in vaccine development. Such changes may or may not be related to attenuation or replicative fitness of the virus. It will be appreciated that such codon substitutions can raise or lower codon pair score, but that the effect relates to the codon pairs that are created in and removed from the sequence, rather than to the frequency of the codon that is substituted. Thus, in a nucleotide sequence in which synonymous codons are shuffled, there may also exist codon substitutions. Even so, codon pair bias can be calculated for the sequence, and reduced codon pair bias reflects virus attenuation. In an embodiment of the invention, a poliovirus protein encoding sequence having a reduced codon pair bias encodes a protein that differs from a natural isolate by 10 amino acids or fewer. In another embodiment of the invention, a poliovirus protein encoding sequence having a reduced codon pair bias encodes a protein that differs from a natural isolate by 20 amino acids or fewer. In another embodiment of the invention, a poliovirus protein encoding sequence is designed, which has a reduced codon pair bias and encodes a designed protein that differs from an initial protein sequence by 10 amino acids or fewer. In another embodiment of the invention, a poliovirus protein encoding sequence is designed, which has a reduced codon pair bias and encodes a designed protein that differs from an initial protein sequence by 20 amino acids or fewer. In certain embodiments, the substitutions are conservative substitutions, as set forth above.

Vaccine Compositions

The present invention provides a vaccine composition for inducing a protective immune response in a subject comprising any of the attenuated viruses described herein and a pharmaceutically acceptable carrier.

It should be understood that an attenuated virus of the invention, where used to elicit a protective immune response in a subject or to prevent a subject from becoming afflicted with a virus-associated disease, is administered to the subject in the form of a composition additionally comprising a pharmaceutically acceptable carrier. Pharmaceutically acceptable carriers are well known to those skilled in the art and include, but are not limited to, one or more of 0.01-0.1M and preferably 0.05M phosphate buffer, phosphate-buffered saline (PBS), or 0.9% saline. Such carriers also include aqueous or non-aqueous solutions, suspensions, and emulsions. Aqueous carriers include water, alcoholic/aqueous solutions, emulsions or suspensions, saline and buffered media. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Solid compositions may comprise nontoxic solid carriers such as, for example, glucose, sucrose, mannitol, sorbitol, lactose, starch, magnesium stearate, cellulose or cellulose derivatives, sodium carbonate and magnesium carbonate. For administration in an aerosol, such as for pulmonary and/or intranasal delivery, an agent or composition is preferably formulated with a nontoxic surfactant, for example, esters or partial esters of C6 to C22 fatty acids or natural glycerides, and a propellant. Additional carriers such as lecithin may be included to facilitate intranasal delivery. Pharmaceutically acceptable carriers can further comprise minor amounts of auxiliary substances such as wetting or emulsifying agents, preservatives and other additives, such as, for example, antimicrobials, antioxidants and chelating agents, which enhance the shelf life and/or effectiveness of the active ingredients. The instant compositions can, as is well known in the art, be formulated so as to provide quick, sustained or delayed release of the active ingredient after administration to a subject.

In various embodiments of the instant vaccine composition, the attenuated virus (i) does not substantially alter the synthesis and processing of viral proteins in an infected cell; (ii) produces similar amounts of virions per infected cell as wt virus; and/or (iii) exhibits substantially lower virion-specific infectivity than wt virus. In further embodiments, the attenuated virus induces a substantially similar immune response in a host animal as the corresponding wt virus.

This invention also provides a modified host cell line specially isolated or engineered to be permissive for an attenuated virus that is inviable in a wild type host cell. Since the attenuated virus cannot grow in normal (wild type) host cells, it is absolutely dependent on the specific helper cell line for growth. This provides a very high level of safety for the generation of virus for vaccine production. Various embodiments of the instant modified cell line permit the growth of an attenuated virus, wherein the genome of said cell line has been altered to increase the number of genes encoding rare tRNAs.

In addition, the present invention provides a method for eliciting a protective immune response in a subject comprising administering to the subject a prophylactically or therapeutically effective dose of any of the vaccine compositions described herein. This invention also provides a method for preventing a subject from becoming afflicted with a virus-associated disease comprising administering to the subject a prophylactically effective dose of any of the instant vaccine compositions. In embodiments of the above methods, the subject has been exposed to a pathogenic virus. “Exposed” to a pathogenic virus means contact with the virus such that infection could result.

The invention further provides a method for delaying the onset, or slowing the rate of progression, of a virus-associated disease in a virus-infected subject comprising administering to the subject a therapeutically effective dose of any of the instant vaccine compositions.

As used herein, “administering” means delivering using any of the various methods and delivery systems known to those skilled in the art. Administering can be performed, for example, intraperitoneally, intracerebrally, intravenously, orally, transmucosally, subcutaneously, transdermally, intradermally, intramuscularly, topically, parenterally, via implant, intrathecally, intralymphatically, intralesionally, pericardially, or epidurally. An agent or composition may also be administered in an aerosol, such as for pulmonary and/or intranasal delivery. Administering may be performed, for example, once, a plurality of times, and/or over one or more extended periods.

Eliciting a protective immune response in a subject can be accomplished, for example, by administering a primary dose of a vaccine to a subject, followed after a suitable period of time by one or more subsequent administrations of the vaccine. A suitable period of time between administrations of the vaccine may readily be determined by one skilled in the art, and is usually on the order of several weeks to months. The present invention is not limited, however, to any particular method, route or frequency of administration.

A “subject” means any animal or artificially modified animal. Animals include, but are not limited to, humans, non-human primates, cows, horses, sheep, pigs, dogs, cats, rabbits, ferrets, rodents such as mice, rats and guinea pigs, and birds. Artificially modified animals include, but are not limited to, SCID mice with human immune systems, and CD155tg transgenic mice expressing the human poliovirus receptor CD155. In a preferred embodiment, the subject is a human. Preferred embodiments of birds are domesticated poultry species, including, but not limited to, chickens, turkeys, ducks, and geese.

A “prophylactically effective dose” is any amount of a vaccine that, when administered to a subject prone to viral infection or prone to affliction with a virus-associated disorder, induces in the subject an immune response that protects the subject from becoming infected by the virus or afflicted with the disorder. “Protecting” the subject means either reducing the likelihood of the subject's becoming infected with the virus, or lessening the likelihood of the disorder's onset in the subject, by at least two-fold, preferably at least ten-fold. For example, if a subject has a 1% chance of becoming infected with a virus, a two-fold reduction in the likelihood of the subject becoming infected with the virus would result in the subject having a 0.5% chance of becoming infected with the virus. Most preferably, a “prophylactically effective dose” induces in the subject an immune response that completely prevents the subject from becoming infected by the virus or prevents the onset of the disorder in the subject entirely.

As used herein, a “therapeutically effective dose” is any amount of a vaccine that, when administered to a subject afflicted with a disorder against which the vaccine is effective, induces in the subject an immune response that causes the subject to experience a reduction, remission or regression of the disorder and/or its symptoms. In preferred embodiments, recurrence of the disorder and/or its symptoms is prevented. In other preferred embodiments, the subject is cured of the disorder and/or its symptoms.

Certain embodiments of any of the instant immunization and therapeutic methods further comprise administering to the subject at least one adjuvant. An “adjuvant” shall mean any agent suitable for enhancing the immunogenicity of an antigen and boosting an immune response in a subject. Numerous adjuvants, including particulate adjuvants, suitable for use with both protein- and nucleic acid-based vaccines, and methods of combining adjuvants with antigens, are well known to those skilled in the art. Suitable adjuvants for nucleic acid based vaccines include, but are not limited to, Quil A, imiquimod, resiquimod, and interleukin-12 delivered in purified protein or nucleic acid form. Adjuvants suitable for use with protein immunization include, but are not limited to, alum, Freund's incomplete adjuvant (FIA), saponin, Quil A, and QS-21.

The invention also provides a kit for immunization of a subject with an attenuated virus of the invention. The kit comprises the attenuated virus, a pharmaceutically acceptable carrier, an applicator, and an instructional material for the use thereof. In further embodiments, the attenuated virus may be one or more poliovirus, one or more rhinovirus, one or more influenza virus, etc. More than one virus may be prefered where it is desirable to immunize a host against a number of different isolates of a particuler virus. The invention includes other embodiments of kits that are known to those skilled in the art. The instructions can provide any information that is useful for directing the administration of the attenuated viruses.

Throughout this application, various publications, reference texts, textbooks, technical manuals, patents, and patent applications have been referred to. The teachings and disclosures of these publications, patents, patent applications and other documents in their entireties are hereby incorporated by reference into this application to more fully describe the state of the art to which the present invention pertains. However, the citation of a reference herein should not be construed as an acknowledgement that such reference is prior art to the present invention.

It is to be understood and expected that variations in the principles of invention herein disclosed can be made by one skilled in the art and it is intended that such modifications are to be included within the scope of the present invention. The following Examples further illustrate the invention, but should not be construed to limit the scope of the invention in any way. Detailed descriptions of conventional methods, such as those employed in the construction of recombinant plasmids, transfection of host cells with viral constructs, polymerase chain reaction (PCR), and immunological techniques can be obtained from numerous publications, including Sambrook et al. (1989) and Coligan et al. (1994). All references mentioned herein are incorporated in their entirety by reference into this application.

### Example 1

### Specific Infectivity and Attenuation of Poliovirus and Recombinant Vaccine Candidates

Specific infectivity and attenuation were compared for recombinant poliovirus vaccine candidates and PVM wild type poliovirus. Viruses were grown on HeLa cells. Virus particle concentration was determined by optical density (1 OD₂₆₀=9.4×10¹² particles/ml). Plaque forming units/ml were determined by a HeLa (R19) plaque assay, and the ratio of virus particles to PFUs were calculated. Relative specific infectivity was determined by normalizing particles/PFU to the wild type value. Viruses were administered to CD155 transgenic mice (which express the PV receptor) by intracerebral injection, and the dose that caused paralysis in 50% of the mice (PLD₅₀) was determined.

Table 3 shows that the engineering of the cre element in the gnome of wt PV decreases dramatically the neurovirulance and relative specific infectivity of the virus. Remarkably, the characterization of PV 1-X shows that the deoptimization of the first one-third of the PV genome does not confer a neuroattenuated phenotype, but reduces the relative specific infectivity of the deoptimized virus.

Table 3 shows that PV1-Mono-cre-X produced about three-fold more virus particles per plaque forming unit than either attenuated parent. In addition, the relative specific infectivity was significantly decreased relative to the parent attenuated viruses. This result indicates that there is a cooperative effect between the cre element and deoptimized P1 on decreasing the relative specific infectivity of highly neuroattenuated PV1-Mono-cre-X virus.

TABLE 3
Specific Infectivity and Attenuation of Recombinant Vaccines.

| | | | | | Relative | |
|---|---|---|---|---|---|---|
| | Absorbance | Purified | Purified | Purified | specific | PLD₅₀ |
| Virus | at 260 nm | particles/ml | PFUs/ml | particles/PFU | infectivity | (PFUs) |
| PVM | 0.956 | 8.97 × 10¹² | 6.0 × 10¹⁰ | 150 | 1.00 | 6.7 × 10¹ |
| PV1-Mono-cre | 0.530 | 4.987 × 10¹² | 8.25 × 10⁹ | 604 | 0.25 | >10⁸ |
| PV1-Xₘᵢₙ | 1.776 | 1.67 × 10¹³ | 3.0 × 10¹⁰ | 556 | 0.27 | 5.0 × 10¹ |
| PV1-Mono-cre-X | 0.667 | 6.27 × 10¹² | 3.6 × 10⁹ | 1739 | 0.084 | >10⁸ |

### Example 2

### Viruses with Reversion Mutations are Neuroattenuated

Neuroattenuation of poliovirus typically results from mutations at a small number of positions. For example, a point mutation within the IBES of Sabin poliovirus vaccine strains is a determinant of the attenuation phenotype. Consequently, such attenuated polioviruses frequently revert to a fully neurovirulent wild type phenotype. Upon repeated passages in neuronal cells, PV1-Mono-cre virus (Ld₅₀>10⁸) and PV1-Mono-cre-X virus (Ld₅₀>10⁸) show an A₁₃₃G mutation, which increase the neuropathogenicity in mice, although the mutated viruses are still attenuated compared with wt poliovirus (LD₅₀=10^{1.9}). The LD₅₀ of A₁₃₃G PV1-Mono-cre virus and A₁₃₃G PV1-Mono-cre-X virus is 10^{4.5} and 10^{5.6}, respectively.

The relative specific infectivity of PV1-Mono-cre virus and PV1-Mono-cre-X virus is 0.25 and 0.084, respectively. Meanwhile, the relative specific infectivity of A₁₃₃G PV1-Mono-cre virus and A₁₃₃G PV1-Mono-cre-X virus is 0.44 and 0.25, respectively. These data indicate that A₁₃₃G mutations also correlate with an increase in the specific infectivity of mutated viruses, although still lower than the wt PV1 (relative specific infectivity=1).

Altogether, these results indicate that if reversion may occur, in the revertant virus, the deoptimization of PV1-Mono-cre virus ameliorates the effect of A133G reversion since A₁₃₃G PV1-Mono-cre-X virus is 1 log₁₀ more attenuated than A₁₃₃G PV1-Mono-cre virus and the relative specific infectivity of mutated deoptimized virus is lower than the A₁₃₃G PV-Mono-cre virus. More important, our data imply that if a reversion happens, the revertant virus of PV1-mono-cre-X will be still highly neuroattenuated, in contrast to what has been observed with Sabin strains which revert to a fully wild type neurovirulence phenotype.

### Example 3

### Construction of Plasmids and DNA Manipulation

The neurovirulent poliovirus type 1 (Mahoney) was the strain used in the laboratory (Cello, 2002). The poliovirus cDNA sequence was that used by Cello et al. (2002) for cDNA synthesis (plasmid pT7PVM) (van der Werf, et al., 1986, Proc Natl Acad Sci USA 83:2330-4). “pT7PVM cre(2C^{ATPase}) mutant” is a full-length poliovirus cDNA clone in which the native cre element in the 2C^{ATPase} coding region was inactivated by introducing three mutations at nt 4462 (G to A), 4465 (C to U), and 4472 (A to C) (Yin, et al., 2003, J. Virol. 77:5152-66; Paul, 2003, In: Semler B L, Wimmer E, editors. Molecular biology of picornaviruses. Washington (DC): ASM Press; 2002. p. 227-46; Rieder, et al., 2000, J. Virol. 74:10371-80). Dual-cre PV is a derivative of pT7PVM carrying two active cre elements; one at nt 102/103 of the 5′-NTR at which a new Nhe I restriction site was created. The second cre element is in the 2C^{ATPase} coding region (FIG. 1A). Mono-crePV has the active cre in the spacer region whereas the native cre in the 2C^{ATPase} coding region has been inactivated (FIG. 1A). FIG. 4 shows the structure of the mono-crePV genome: The single-stranded RNA is covalently linked to the viral-encoded protein VPg at the 5′ end of the non-translated region (5′-NTR). The 5′-NTR consists of two cis-acting domains, the cloverleaf and the internal ribosomal entry site (IRES), which are separated by a spacer region. The IRES controls translation of the polyprotein (open box), consisting of structural (P1) region and nonstructural regions (P2 and P3), specifying the replication proteins. Within the 2C^{ATPase} coding region, the cis replication element (cre) is indicated. The 3′-NTR contains a heteropolymeric region and is polyadenylylated. RNA replication requires all three structural elements, cloverleaf, cre and the 3′-NTR. The native cre in 2C^{ATPase} was inactivated by mutation as indicated by an X (mono-crePV).

### Example 4

### In Vitro Transcription, Transfection and One-Step Growth Curves

All plasmids were linearized with DraI. RNAs were synthesized with phage T7 RNA polymerase, and the RNA transcripts were transfected into HeLa cell monolayers by the DEAE-dextran method as described previously (van der Werf, 1986). The incubation time was up to 2 days and virus titers were determined by a plaque assay (Pincus, et al., 1986, J. Virol. 57: 638-46.). One-step growth curves in HeLa, MRC5, and 293T cells were carried out as follows. Cell monolayers (1×10⁶ cells) were infected at a multiplicity of infection (MOI) of 10. The plates were incubated at 33° C., 37° C. or 39.5° C., as indicated, and the cells were harvested at 0, 2, 4, 6, 8, 12, 24, 48, and 72 h post infection. The plates were subjected to three consecutive freeze-thaw cycles, and the viral titers of the supernatants were determined by plaque assay on HeLa cell monolayers, as describe before (Pincus, 1986).

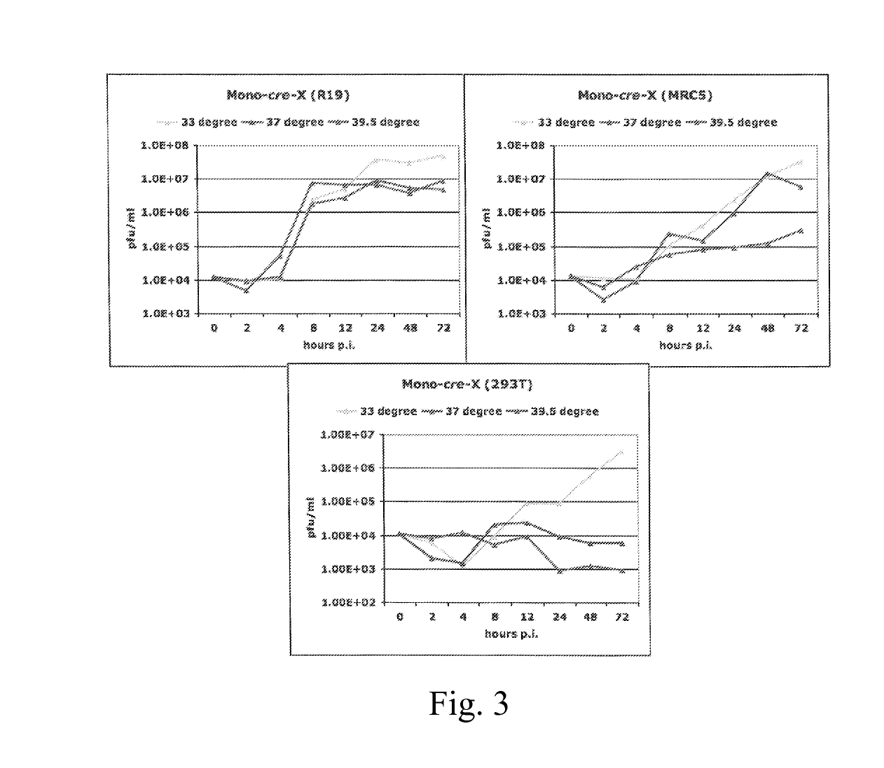

Results are shown in FIG. 3. Mono-cre-X replicated efficiently at all three temperatures in HeLa cells. High titers of Mono-cre-X were also attained at 33° C. and 37° C. using MRC5 but replication was reduced at 39.5° C. In 293T cells, replication was strongly restricted at 37° C. and 39.5° C.

SUPPLEMENTAL TABLE 1
Expected and Observed Codon Pair Frequencies and
Codon Pair Scores for 14,795 Human Genes

| amino | | | | | |
|---|---|---|---|---|---|
| acid | codon | | | observed / | |
| pair | pair | expected | observed | expected | CPS |
| AA | GCGGCG | 630.04 | 2870 | 4.555 | 1.516 |
| AA | GCGGCC | 2330.20 | 4032 | 1.730 | 0.548 |
| AA | GCTGCT | 3727.41 | 5562 | 1.492 | 0.400 |
| AA | GCAGCA | 2856.40 | 4196 | 1.469 | 0.385 |
| AA | GCAGCT | 3262.97 | 4711 | 1.444 | 0.367 |
| AA | GCTGCA | 3262.97 | 4357 | 1.335 | 0.289 |
| AA | GCTGCC | 5667.77 | 7014 | 1.238 | 0.213 |
| AA | GCAGCC | 4961.56 | 6033 | 1.216 | 0.196 |
| AA | GCAGCG | 1341.51 | 1420 | 1.059 | 0.057 |
| AA | GCTGCG | 1532.46 | 1533 | 1.000 | 0.000 |
| AA | GCGGCT | 1532.46 | 1472 | 0.961 | −0.040 |
| AA | GCCGCG | 2330.20 | 2042 | 0.876 | −0.132 |
| AA | GCGGCA | 1341.51 | 1142 | 0.851 | −0.161 |
| AA | GCCGCC | 8618.21 | 5141 | 0.597 | −0.517 |
| AA | GCCGCT | 5667.77 | 1378 | 0.243 | −1.414 |
| AA | GCCGCA | 4961.56 | 1122 | 0.226 | −1.487 |
| AC | GCCTGC | 2333.61 | 3975 | 1.703 | 0.533 |
| AC | GCCTGT | 1965.56 | 2436 | 1.239 | 0.215 |
| AC | GCGTGC | 630.96 | 560 | 0.888 | −0.119 |
| AC | GCTTGT | 1292.65 | 1142 | 0.883 | −0.124 |
| AC | GCATGT | 1131.59 | 881 | 0.779 | −0.250 |
| AC | GCGTGT | 531.45 | 322 | 0.606 | −0.501 |
| AC | GCTTGC | 1534.70 | 894 | 0.583 | −0.540 |
| AC | GCATGC | 1343.47 | 554 | 0.412 | −0.886 |
| AD | GCAGAT | 2373.33 | 4215 | 1.776 | 0.574 |
| AD | GCTGAT | 2711.15 | 3887 | 1.434 | 0.360 |
| AD | GCTGAC | 3062.55 | 4374 | 1.428 | 0.356 |
| AD | GCGGAC | 1259.11 | 1625 | 1.291 | 0.255 |
| AD | GCAGAC | 2680.95 | 3395 | 1.266 | 0.236 |
| AD | GCGGAT | 1114.64 | 839 | 0.753 | −0.284 |
| AD | GCCGAC | 4656.80 | 2726 | 0.585 | −0.535 |
| AD | GCCGAT | 4122.47 | 920 | 0.223 | −1.500 |
| AE | GCAGAA | 3517.48 | 5814 | 1.653 | 0.503 |
| AE | GCAGAG | 4703.98 | 7094 | 1.508 | 0.411 |
| AE | GCGGAG | 2209.23 | 3171 | 1.435 | 0.361 |
| AE | GCTGAG | 5373.53 | 7362 | 1.370 | 0.315 |
| AE | GCTGAA | 4018.14 | 5186 | 1.291 | 0.255 |
| AE | GCCGAG | 8170.80 | 5082 | 0.622 | −0.475 |
| AE | GCGGAA | 1651.99 | 949 | 0.574 | −0.554 |
| AE | GCCGAA | 6109.85 | 1097 | 0.180 | −1.717 |
| AF | GCCTTC | 4447.90 | 7382 | 1.660 | 0.507 |
| AF | GCATTT | 2237.22 | 2332 | 1.042 | 0.041 |
| AF | GCTTTT | 2555.66 | 2580 | 1.010 | 0.009 |
| AF | GCCTTT | 3886.04 | 3842 | 0.989 | −0.011 |
| AF | GCTTTC | 2925.16 | 2315 | 0.791 | −0.234 |
| AF | GCGTTC | 1202.63 | 636 | 0.529 | −0.637 |
| AF | GCGTTT | 1050.71 | 518 | 0.493 | −0.707 |
| AF | GCATTC | 2560.68 | 1261 | 0.492 | −0.708 |
| AG | GCGGGC | 1369.64 | 2638 | 1.926 | 0.655 |
| AG | GCGGGG | 986.17 | 1738 | 1.762 | 0.567 |
| AG | GCTGGG | 2398.67 | 3855 | 1.607 | 0.474 |
| AG | GCTGGT | 1590.73 | 2524 | 1.587 | 0.462 |
| AG | GCTGGA | 2457.02 | 3783 | 1.540 | 0.432 |
| AG | GCAGGA | 2150.87 | 3074 | 1.429 | 0.357 |
| AG | GCAGGG | 2099.79 | 2782 | 1.325 | 0.281 |
| AG | GCAGGT | 1392.52 | 1748 | 1.255 | 0.227 |
| AG | GCTGGC | 3331.38 | 3961 | 1.189 | 0.173 |
| AG | GCAGGC | 2916.28 | 3119 | 1.070 | 0.067 |
| AG | GCGGGT | 654.00 | 617 | 0.943 | −0.058 |
| AG | GCGGGA | 1010.16 | 793 | 0.785 | −0.242 |
| AG | GCCGGG | 3647.33 | 2240 | 0.614 | −0.488 |
| AG | GCCGGC | 5065.58 | 2977 | 0.588 | −0.532 |
| AG | GCCGGT | 2418.80 | 581 | 0.240 | −1.426 |
| AG | GCCGGA | 3736.06 | 795 | 0.213 | −1.547 |
| AH | GCGCAC | 748.29 | 983 | 1.314 | 0.273 |
| AH | GCCCAC | 2767.53 | 3465 | 1.252 | 0.225 |
| AH | GCTCAT | 1319.86 | 1471 | 1.115 | 0.108 |
| AH | GCACAT | 1155.40 | 1122 | 0.971 | −0.029 |
| AH | GCCCAT | 2006.93 | 1827 | 0.910 | −0.094 |
| AH | GCTCAC | 1820.07 | 1526 | 0.838 | −0.176 |
| AH | GCACAC | 1593.29 | 1312 | 0.823 | −0.194 |
| AH | GCGCAT | 542.64 | 248 | 0.457 | −0.783 |
| AI | GCCATC | 3894.51 | 7798 | 2.002 | 0.694 |
| AI | GCCATT | 3079.73 | 3761 | 1.221 | 0.200 |
| AI | GCAATA | 815.43 | 924 | 1.133 | 0.125 |
| AI | GCAATT | 1773.02 | 1684 | 0.950 | −0.052 |
| AI | GCCATA | 1416.41 | 1257 | 0.887 | −0.119 |
| AI | GCTATT | 2025.39 | 1709 | 0.844 | −0.170 |
| AI | GCTATA | 931.50 | 771 | 0.828 | −0.189 |
| AI | GCTATC | 2561.23 | 1194 | 0.466 | −0.763 |
| AI | GCGATT | 832.70 | 373 | 0.448 | −0.803 |
| AI | GCAATC | 2242.09 | 984 | 0.439 | −0.824 |
| AI | GCGATA | 382.97 | 149 | 0.389 | −0.944 |
| AI | GCGATC | 1053.00 | 404 | 0.384 | −0.958 |
| AK | GCCAAG | 5767.01 | 9818 | 1.702 | 0.532 |
| AK | GCAAAA | 2563.57 | 3011 | 1.175 | 0.161 |
| AK | GCCAAA | 4452.91 | 4794 | 1.077 | 0.074 |
| AK | GCAAAG | 3320.10 | 3044 | 0.917 | −0.087 |
| AK | GCTAAA | 2928.46 | 2022 | 0.690 | −0.370 |
| AK | GCGAAG | 1559.29 | 765 | 0.491 | −0.712 |
| AK | GCTAAG | 3792.68 | 1725 | 0.455 | −0.788 |
| AK | GCGAAA | 1203.98 | 409 | 0.340 | −1.080 |
| AL | GCGCTG | 2369.16 | 4619 | 1.950 | 0.668 |
| AL | GCGCTC | 1140.05 | 1765 | 1.548 | 0.437 |
| AL | GCTTTG | 1873.51 | 2601 | 1.388 | 0.328 |
| AL | GCCCTG | 8762.30 | 11409 | 1.302 | 0.264 |
| AL | GCCTTG | 2848.79 | 3695 | 1.297 | 0.260 |
| AL | GCTTTA | 1115.24 | 1385 | 1.242 | 0.217 |
| AL | GCCCTC | 4216.45 | 4499 | 1.067 | 0.065 |
| AL | GCTCTT | 1912.07 | 2038 | 1.066 | 0.064 |
| AL | GCATTA | 976.28 | 986 | 1.010 | 0.010 |
| AL | GCTCTA | 1031.16 | 940 | 0.912 | −0.093 |
| AL | GCACTT | 1673.82 | 1444 | 0.863 | −0.148 |
| AL | GCATTG | 1640.07 | 1364 | 0.832 | −0.184 |
| AL | GCACTA | 902.68 | 747 | 0.828 | −0.189 |
| AL | GCGCTA | 423.94 | 342 | 0.807 | −0.215 |
| AL | GCCCTA | 1567.95 | 1228 | 0.783 | −0.244 |
| AL | GCTCTG | 5762.53 | 4505 | 0.782 | −0.246 |
| AL | GCCCTT | 2907.42 | 2230 | 0.767 | −0.265 |
| AL | GCTCTC | 2772.95 | 2036 | 0.734 | −0.309 |
| AL | GCCTTA | 1695.80 | 1205 | 0.711 | −0.342 |
| AL | GCACTG | 5044.51 | 3522 | 0.698 | −0.359 |
| AL | GCGTTG | 770.26 | 476 | 0.618 | −0.481 |
| AL | GCGCTT | 786.11 | 459 | 0.584 | −0.538 |
| AL | GCACTC | 2427.43 | 1415 | 0.583 | −0.540 |
| AL | GCGTTA | 458.51 | 169 | 0.369 | −0.998 |
| AM | GCCATG | 4236.47 | 6521 | 1.539 | 0.431 |
| AM | GCAATG | 2438.96 | 1900 | 0.779 | −0.250 |
| AM | GCTATG | 2786.11 | 1561 | 0.560 | −0.579 |
| AM | GCGATG | 1145.46 | 625 | 0.546 | −0.606 |
| AN | GCCAAC | 3190.28 | 5452 | 1.709 | 0.536 |
| AN | GCAAAT | 1667.60 | 2282 | 1.368 | 0.314 |
| AN | GCCAAT | 2896.62 | 3122 | 1.078 | 0.075 |
| AN | GCAAAC | 1836.66 | 1512 | 0.823 | −0.195 |
| AN | GCTAAT | 1904.97 | 1356 | 0.712 | −0.340 |
| AN | GCTAAC | 2098.09 | 925 | 0.441 | −0.819 |
| AN | GCGAAC | 862.59 | 331 | 0.384 | −0.958 |
| AN | GCGAAT | 783.19 | 260 | 0.332 | −1.103 |
| AP | GCGCCG | 406.74 | 1172 | 2.881 | 1.058 |
| AP | GCGCCC | 1122.56 | 2271 | 2.023 | 0.705 |
| AP | GCCCCG | 1504.34 | 2335 | 1.552 | 0.440 |
| AP | GCTCCA | 2360.19 | 2463 | 1.044 | 0.043 |
| AP | GCTCCT | 2445.47 | 2548 | 1.042 | 0.041 |
| AP | GCCCCC | 4151.78 | 3957 | 0.953 | −0.048 |
| AP | GCACCT | 2140.76 | 2028 | 0.947 | −0.054 |
| AP | GCCCCA | 3588.82 | 3371 | 0.939 | −0.063 |
| AP | GCACCA | 2066.10 | 1831 | 0.886 | −0.121 |
| AP | GCACCC | 2390.20 | 2111 | 0.883 | −0.124 |
| AP | GCCCCT | 3718.49 | 3269 | 0.879 | −0.129 |
| AP | GCTCCC | 2730.42 | 2384 | 0.873 | −0.136 |
| AP | GCTCCG | 989.33 | 773 | 0.781 | −0.247 |
| AP | GCGCCT | 1005.41 | 778 | 0.774 | −0.256 |
| AP | GCACCG | 866.06 | 571 | 0.659 | −0.417 |
| AP | GCGCCA | 970.35 | 595 | 0.613 | −0.489 |
| AQ | GCCCAG | 7143.67 | 9550 | 1.337 | 0.290 |
| AQ | GCGCAG | 1931.51 | 2101 | 1.088 | 0.084 |
| AQ | GCACAA | 1472.79 | 1416 | 0.961 | −0.039 |
| AQ | GCTCAA | 1682.42 | 1522 | 0.905 | −0.100 |
| AQ | GCTCAG | 4698.04 | 4141 | 0.881 | −0.126 |
| AQ | GCACAG | 4112.65 | 3374 | 0.820 | −0.198 |
| AQ | GCCCAA | 2558.23 | 1943 | 0.760 | −0.275 |
| AQ | GCGCAA | 691.70 | 244 | 0.353 | −1.042 |
| AR | GCGCGC | 580.17 | 1255 | 2.163 | 0.772 |
| AR | GCGCGG | 634.54 | 1175 | 1.852 | 0.616 |
| AR | GCCCGG | 2346.82 | 3946 | 1.681 | 0.520 |
| AR | GCCCGC | 2145.76 | 3135 | 1.461 | 0.379 |
| AR | GCCAGG | 2323.57 | 3242 | 1.395 | 0.333 |
| AR | GCAAGA | 1362.59 | 1559 | 1.144 | 0.135 |
| AR | GCTCGA | 836.64 | 943 | 1.127 | 0.120 |
| AR | GCCCGA | 1272.16 | 1418 | 1.115 | 0.109 |
| AR | GCCCGT | 918.67 | 935 | 1.018 | 0.018 |
| AR | GCTCGT | 604.17 | 595 | 0.985 | −0.015 |
| AR | GCCAGA | 2366.81 | 2219 | 0.938 | −0.064 |
| AR | GCTCGG | 1543.39 | 1295 | 0.839 | −0.175 |
| AR | GCGCGT | 248.39 | 205 | 0.825 | −0.192 |
| AR | GCAAGG | 1337.69 | 1089 | 0.814 | −0.206 |
| AR | GCGAGG | 628.25 | 486 | 0.774 | −0.257 |
| AR | GCACGA | 732.39 | 533 | 0.728 | −0.318 |
| AR | GCTCGC | 1411.16 | 941 | 0.667 | −0.405 |
| AR | GCGCGA | 343.97 | 226 | 0.657 | −0.420 |
| AR | GCACGT | 528.89 | 338 | 0.639 | −0.448 |
| AR | GCACGG | 1351.08 | 859 | 0.636 | −0.453 |
| AR | GCACGC | 1235.33 | 619 | 0.501 | −0.691 |
| AR | GCTAGA | 1556.53 | 714 | 0.459 | −0.779 |
| AR | GCGAGA | 639.94 | 263 | 0.411 | −0.889 |
| AR | GCTAGG | 1528.10 | 487 | 0.319 | −1.144 |
| AS | GCCTCG | 963.41 | 1977 | 2.052 | 0.719 |
| AS | GCGTCG | 260.49 | 465 | 1.785 | 0.579 |
| AS | GCCAGC | 4127.58 | 6466 | 1.567 | 0.449 |
| AS | GCCTCC | 3643.21 | 5443 | 1.494 | 0.401 |
| AS | GCTTCT | 2084.25 | 2488 | 1.194 | 0.177 |
| AS | GCCAGT | 2604.12 | 3085 | 1.185 | 0.169 |
| AS | GCATCT | 1824.55 | 2154 | 1.181 | 0.166 |
| AS | GCTTCA | 1684.99 | 1932 | 1.147 | 0.137 |
| AS | GCGTCC | 985.05 | 1079 | 1.095 | 0.091 |
| AS | GCATCA | 1475.04 | 1531 | 1.038 | 0.037 |
| AS | GCCTCT | 3169.23 | 3235 | 1.021 | 0.021 |
| AS | GCCTCA | 2562.14 | 2514 | 0.981 | −0.019 |
| AS | GCTTCC | 2395.96 | 2295 | 0.958 | −0.043 |
| AS | GCAAGT | 1499.21 | 1307 | 0.872 | −0.137 |
| AS | GCTTCG | 633.59 | 516 | 0.814 | −0.205 |
| AS | GCATCC | 2097.42 | 1658 | 0.790 | −0.235 |
| AS | GCATCG | 554.64 | 403 | 0.727 | −0.319 |
| AS | GCGTCT | 856.90 | 521 | 0.608 | −0.498 |
| AS | GCGAGC | 1116.02 | 595 | 0.533 | −0.629 |
| AS | GCGTCA | 692.75 | 319 | 0.460 | −0.775 |
| AS | GCAAGC | 2376.27 | 1080 | 0.454 | −0.789 |
| AS | GCTAGT | 1712.60 | 737 | 0.430 | −0.843 |
| AS | GCGAGT | 704.10 | 265 | 0.376 | −0.977 |
| AS | GCTAGC | 2714.51 | 673 | 0.248 | −1.395 |
| AT | GCCACG | 1262.40 | 2478 | 1.963 | 0.674 |
| AT | GCCACC | 3842.98 | 6598 | 1.717 | 0.541 |
| AT | GCCACA | 3111.04 | 4031 | 1.296 | 0.259 |
| AT | GCCACT | 2751.18 | 3205 | 1.165 | 0.153 |
| AT | GCAACA | 1791.05 | 1761 | 0.983 | −0.017 |
| AT | GCGACG | 341.33 | 329 | 0.964 | −0.037 |
| AT | GCAACT | 1583.87 | 1509 | 0.953 | −0.048 |
| AT | GCTACT | 1809.31 | 1395 | 0.771 | −0.260 |
| AT | GCTACA | 2045.98 | 1528 | 0.747 | −0.292 |
| AT | GCGACC | 1039.07 | 601 | 0.578 | −0.547 |
| AT | GCAACC | 2212.43 | 1259 | 0.569 | −0.564 |
| AT | GCTACC | 2527.34 | 1364 | 0.540 | −0.617 |
| AT | GCAACG | 726.77 | 384 | 0.528 | −0.638 |
| AT | GCTACG | 830.22 | 363 | 0.437 | −0.827 |
| AT | GCGACT | 743.87 | 308 | 0.414 | −0.882 |
| AT | GCGACA | 841.17 | 347 | 0.413 | −0.885 |
| AV | GCTGTT | 1736.99 | 3025 | 1.742 | 0.555 |
| AV | GCTGTG | 4399.56 | 7279 | 1.654 | 0.503 |
| AV | GCTGTA | 1127.89 | 1750 | 1.552 | 0.439 |
| AV | GCTGTC | 2223.90 | 3351 | 1.507 | 0.410 |
| AV | GCAGTA | 987.35 | 1401 | 1.419 | 0.350 |
| AV | GCGGTG | 1808.80 | 2487 | 1.375 | 0.318 |
| AV | GCAGTT | 1520.56 | 2087 | 1.373 | 0.317 |
| AV | GCAGTG | 3851.36 | 4349 | 1.129 | 0.122 |
| AV | GCGGTC | 914.32 | 883 | 0.966 | −0.035 |
| AV | GCAGTC | 1946.80 | 1806 | 0.928 | −0.075 |
| AV | GCCGTG | 6689.81 | 4322 | 0.646 | −0.437 |
| AV | GCGGTT | 714.13 | 423 | 0.592 | −0.524 |
| AV | GCGGTA | 463.71 | 270 | 0.582 | −0.541 |
| AV | GCCGTC | 3381.59 | 1798 | 0.532 | −0.632 |
| AV | GCCGTT | 2641.21 | 563 | 0.213 | −1.546 |
| AV | GCCGTA | 1715.03 | 329 | 0.192 | −1.651 |
| AW | GCCTGG | 2528.22 | 3848 | 1.522 | 0.420 |
| AW | GCGTGG | 683.58 | 558 | 0.816 | −0.203 |
| AW | GCTTGG | 1662.69 | 1066 | 0.641 | −0.445 |
| AW | GCATGG | 1455.51 | 858 | 0.589 | −0.529 |
| AY | GCCTAC | 2643.77 | 4073 | 1.541 | 0.432 |
| AY | GCCTAT | 2148.26 | 2457 | 1.144 | 0.134 |
| AY | GCTTAT | 1412.81 | 1478 | 1.046 | 0.045 |
| AY | GCATAT | 1236.77 | 1244 | 1.006 | 0.006 |
| AY | GCTTAC | 1738.68 | 1139 | 0.655 | −0.423 |
| AY | GCGTAC | 714.83 | 429 | 0.600 | −0.511 |
| AY | GCATAC | 1522.04 | 868 | 0.570 | −0.562 |
| AY | GCGTAT | 580.85 | 310 | 0.534 | −0.628 |
| CA | TGTGCT | 1164.04 | 2021 | 1.736 | 0.552 |
| CA | TGTGCC | 1769.99 | 2992 | 1.690 | 0.525 |
| CA | TGTGCA | 1019.00 | 1708 | 1.676 | 0.517 |
| CA | TGTGCG | 478.57 | 477 | 0.997 | −0.003 |
| CA | TGCGCG | 568.18 | 502 | 0.884 | −0.124 |
| CA | TGCGCC | 2101.42 | 1313 | 0.625 | −0.470 |
| CA | TGCGCT | 1382.00 | 368 | 0.266 | −1.323 |
| CA | TGCGCA | 1209.80 | 312 | 0.258 | −1.355 |
| CC | TGCTGC | 1534.17 | 2610 | 1.701 | 0.531 |
| CC | TGCTGT | 1292.21 | 1571 | 1.216 | 0.195 |
| CC | TGTTGT | 1088.41 | 529 | 0.486 | −0.721 |
| CC | TGTTGC | 1292.21 | 497 | 0.385 | −0.956 |
| CD | TGTGAC | 1920.20 | 3470 | 1.807 | 0.592 |
| CD | TGTGAT | 1699.87 | 2853 | 1.678 | 0.518 |
| CD | TGCGAC | 2279.75 | 1134 | 0.497 | −0.698 |
| CD | TGCGAT | 2018.17 | 461 | 0.228 | −1.477 |
| CE | TGTGAA | 1901.69 | 3636 | 1.912 | 0.648 |
| CE | TGTGAG | 2543.16 | 3935 | 1.547 | 0.437 |
| CE | TGCGAG | 3019.37 | 1709 | 0.566 | −0.569 |
| CE | TGCGAA | 2257.78 | 442 | 0.196 | −1.631 |
| CF | TGCTTC | 1891.74 | 2684 | 1.419 | 0.350 |
| CF | TGCTTT | 1652.78 | 1685 | 1.019 | 0.019 |
| CF | TGTTTT | 1392.11 | 1096 | 0.787 | −0.239 |
| CF | TGTTTC | 1593.38 | 1065 | 0.668 | −0.403 |
| CG | TGTGGG | 1594.78 | 3240 | 2.032 | 0.709 |
| CG | TGTGGA | 1633.57 | 2846 | 1.742 | 0.555 |
| CG | TGTGGT | 1057.61 | 1627 | 1.538 | 0.431 |
| CG | TGTGGC | 2214.90 | 3133 | 1.415 | 0.347 |
| CG | TGCGGG | 1893.40 | 1137 | 0.601 | −0.510 |
| CG | TGCGGC | 2629.63 | 1461 | 0.556 | −0.588 |
| CG | TGCGGT | 1255.64 | 344 | 0.274 | −1.295 |
| CG | TGCGGA | 1939.46 | 431 | 0.222 | −1.504 |
| CH | TGCCAC | 1618.50 | 2144 | 1.325 | 0.281 |
| CH | TGCCAT | 1173.68 | 1253 | 1.068 | 0.065 |
| CH | TGTCAT | 988.58 | 831 | 0.841 | −0.174 |
| CH | TGTCAC | 1363.24 | 916 | 0.672 | −0.398 |
| CI | TGCATC | 1821.04 | 2813 | 1.545 | 0.435 |
| CI | TGCATT | 1440.05 | 1579 | 1.096 | 0.092 |
| CI | TGCATA | 662.30 | 576 | 0.870 | −0.140 |
| CI | TGTATA | 557.84 | 474 | 0.850 | −0.163 |
| CI | TGTATT | 1212.94 | 927 | 0.764 | −0.269 |
| CI | TGTATC | 1533.83 | 859 | 0.560 | −0.580 |
| CK | TGCAAG | 2777.53 | 3348 | 1.205 | 0.187 |
| CK | TGCAAA | 2144.62 | 2441 | 1.138 | 0.129 |
| CK | TGTAAA | 1806.38 | 1770 | 0.980 | −0.020 |
| CK | TGTAAG | 2339.47 | 1509 | 0.645 | −0.438 |
| CL | TGCCTC | 1722.14 | 2468 | 1.433 | 0.360 |
| CL | TGCCTG | 3578.83 | 4525 | 1.264 | 0.235 |
| CL | TGTTTA | 583.38 | 704 | 1.207 | 0.188 |
| CL | TGCCTT | 1187.49 | 1384 | 1.165 | 0.153 |
| CL | TGTTTG | 980.04 | 1079 | 1.101 | 0.096 |
| CL | TGCTTG | 1163.55 | 1179 | 1.013 | 0.013 |
| CL | TGTCTT | 1000.21 | 940 | 0.940 | −0.062 |
| CL | TGCCTA | 640.41 | 585 | 0.913 | −0.090 |
| CL | TGTCTA | 539.40 | 481 | 0.892 | −0.115 |
| CL | TGCTTA | 692.62 | 565 | 0.816 | −0.204 |
| CL | TGTCTC | 1450.53 | 1010 | 0.696 | −0.362 |
| CL | TGTCTG | 3014.39 | 1633 | 0.542 | −0.613 |
| CM | TGCATG | 1518.22 | 1979 | 1.304 | 0.265 |
| CM | TGTATG | 1278.78 | 818 | 0.640 | −0.447 |
| CN | TGCAAC | 1825.04 | 2351 | 1.288 | 0.253 |
| CN | TGCAAT | 1657.05 | 1636 | 0.987 | −0.013 |
| CN | TGTAAT | 1395.71 | 1349 | 0.967 | −0.034 |
| CN | TGTAAC | 1537.20 | 1079 | 0.702 | −0.354 |
| CP | TGCCCG | 687.28 | 978 | 1.423 | 0.353 |
| CP | TGCCCC | 1896.80 | 2279 | 1.201 | 0.184 |
| CP | TGCCCA | 1639.61 | 1728 | 1.054 | 0.053 |
| CP | TGCCCT | 1698.85 | 1690 | 0.995 | −0.005 |
| CP | TGTCCT | 1430.91 | 1333 | 0.932 | −0.071 |
| CP | TGTCCA | 1381.01 | 1263 | 0.915 | −0.089 |
| CP | TGTCCC | 1597.65 | 1369 | 0.857 | −0.154 |
| CP | TGTCCG | 578.88 | 271 | 0.468 | −0.759 |
| CQ | TGCCAG | 3338.89 | 4321 | 1.294 | 0.258 |
| CQ | TGCCAA | 1195.69 | 1319 | 1.103 | 0.098 |
| CQ | TGTCAA | 1007.11 | 905 | 0.899 | −0.107 |
| CQ | TGTCAG | 2812.30 | 1809 | 0.643 | −0.441 |
| CR | TGCCGC | 1031.52 | 1860 | 1.803 | 0.590 |
| CR | TGCCGG | 1128.18 | 1543 | 1.368 | 0.313 |
| CR | TGCAGG | 1117.00 | 1450 | 1.298 | 0.261 |
| CR | TGCCGT | 441.63 | 541 | 1.225 | 0.203 |
| CR | TGCCGA | 611.56 | 742 | 1.213 | 0.193 |
| CR | TGCAGA | 1137.78 | 1252 | 1.100 | 0.096 |
| CR | TGTCGA | 515.11 | 458 | 0.889 | −0.118 |
| CR | TGTCGT | 371.98 | 308 | 0.828 | −0.189 |
| CR | TGTAGA | 958.34 | 570 | 0.595 | −0.520 |
| CR | TGTCGC | 868.83 | 497 | 0.572 | −0.559 |
| CR | TGTCGG | 950.24 | 463 | 0.487 | −0.719 |
| CR | TGTAGG | 940.83 | 389 | 0.413 | −0.883 |
| CS | TGCAGC | 1990.73 | 3150 | 1.582 | 0.459 |
| CS | TGCTCC | 1757.12 | 2397 | 1.364 | 0.311 |
| CS | TGCAGT | 1255.97 | 1701 | 1.354 | 0.303 |
| CS | TGCTCG | 464.65 | 571 | 1.229 | 0.206 |
| CS | TGTTCT | 1287.45 | 1184 | 0.920 | −0.084 |
| CS | TGCTCT | 1528.52 | 1393 | 0.911 | −0.093 |
| CS | TGTTCA | 1040.83 | 932 | 0.895 | −0.110 |
| CS | TGCTCA | 1235.72 | 1079 | 0.873 | −0.136 |
| CS | TGTTCC | 1479.99 | 1102 | 0.745 | −0.295 |
| CS | TGTAGT | 1057.88 | 699 | 0.661 | −0.414 |
| CS | TGTTCG | 391.37 | 192 | 0.491 | −0.712 |
| CS | TGTAGC | 1676.76 | 767 | 0.457 | −0.782 |
| CT | TGCACG | 535.88 | 829 | 1.547 | 0.436 |
| CT | TGCACC | 1631.31 | 2321 | 1.423 | 0.353 |
| CT | TGCACA | 1320.60 | 1508 | 1.142 | 0.133 |
| CT | TGCACT | 1167.85 | 1185 | 1.015 | 0.015 |
| CT | TGTACT | 983.66 | 802 | 0.815 | −0.204 |
| CT | TGTACA | 1112.32 | 830 | 0.746 | −0.293 |
| CT | TGTACC | 1374.02 | 942 | 0.686 | −0.377 |
| CT | TGTACG | 451.36 | 160 | 0.354 | −1.037 |
| CV | TGTGTC | 1064.94 | 1821 | 1.710 | 0.536 |
| CV | TGTGTT | 831.78 | 1383 | 1.663 | 0.508 |
| CV | TGTGTA | 540.10 | 866 | 1.603 | 0.472 |
| CV | TGTGTG | 2106.78 | 3241 | 1.538 | 0.431 |
| CV | TGCGTG | 2501.27 | 1537 | 0.614 | −0.487 |
| CV | TGCGTC | 1264.35 | 734 | 0.581 | −0.544 |
| CV | TGCGTT | 987.53 | 219 | 0.222 | −1.506 |
| CV | TGCGTA | 641.24 | 137 | 0.214 | −1.543 |
| CW | TGCTGG | 1275.05 | 1842 | 1.445 | 0.368 |
| CW | TGTTGG | 1073.95 | 507 | 0.472 | −0.751 |
| CY | TGCTAC | 1379.34 | 1995 | 1.446 | 0.369 |
| CY | TGCTAT | 1120.82 | 1170 | 1.044 | 0.043 |
| CY | TGTTCT | 944.05 | 653 | 0.692 | −0.369 |
| CY | TGTTCC | 1161.80 | 788 | 0.678 | −0.388 |
| DA | GATGCT | 2675.13 | 5292 | 1.978 | 0.682 |
| DA | GATGCA | 2341.80 | 3898 | 1.665 | 0.510 |
| DA | GATGCC | 4067.71 | 5983 | 1.471 | 0.386 |
| DA | GACGCG | 1242.39 | 1116 | 0.898 | −0.107 |
| DA | GATGCG | 1099.83 | 972 | 0.884 | −0.124 |
| DA | GACGCC | 4594.94 | 2668 | 0.581 | −0.544 |
| DA | GACGCA | 2645.34 | 852 | 0.322 | −1.133 |
| DA | GACGCT | 3021.87 | 908 | 0.300 | −1.202 |
| DC | GACTGC | 2386.86 | 3465 | 1.452 | 0.373 |
| DC | GACTGT | 2010.41 | 2804 | 1.395 | 0.333 |
| DC | GATTGT | 1779.74 | 1163 | 0.653 | −0.425 |
| DC | GATTGC | 2112.99 | 858 | 0.406 | −0.901 |
| DD | GATGAT | 4271.42 | 7846 | 1.837 | 0.608 |
| DD | GATGAC | 4825.06 | 7181 | 1.488 | 0.398 |
| DD | GACGAC | 5450.46 | 2965 | 0.544 | −0.609 |
| DD | GACGAT | 4825.06 | 1380 | 0.286 | −1.252 |
| DE | GATGAA | 5114.33 | 10045 | 1.964 | 0.675 |
| DE | GATGAG | 6839.48 | 9573 | 1.400 | 0.336 |
| DE | GACGAG | 7725.97 | 4498 | 0.582 | −0.541 |
| DE | GACGAA | 5777.22 | 1341 | 0.232 | −1.461 |
| DF | GACTTC | 4696.28 | 6094 | 1.298 | 0.261 |
| DF | GACTTT | 4103.05 | 4250 | 1.036 | 0.035 |
| DF | GATTTT | 3632.26 | 3485 | 0.959 | −0.041 |
| DF | GATTTC | 4157.42 | 2760 | 0.664 | −0.410 |
| DG | GATGGT | 1910.36 | 3443 | 1.802 | 0.589 |
| DG | GATGGA | 2950.72 | 5133 | 1.740 | 0.554 |
| DG | GATGGG | 2880.65 | 4437 | 1.540 | 0.432 |
| DG | GATGGC | 4000.77 | 5419 | 1.354 | 0.303 |
| DG | GACGGC | 4519.33 | 2987 | 0.661 | −0.414 |
| DG | GACGGG | 3254.02 | 1979 | 0.608 | −0.497 |
| DG | GACGGT | 2157.97 | 723 | 0.335 | −1.094 |
| DG | GACGGA | 3333.18 | 886 | 0.266 | −1.325 |
| DH | GACCAC | 2653.74 | 3480 | 1.311 | 0.271 |
| DH | GACCAT | 1924.41 | 2014 | 1.047 | 0.046 |
| DH | GATCAT | 1703.60 | 1623 | 0.953 | −0.048 |
| DH | GATCAC | 2349.25 | 1514 | 0.644 | −0.439 |
| DI | GACATC | 4715.94 | 6532 | 1.385 | 0.326 |
| DI | GACATT | 3729.31 | 4087 | 1.096 | 0.092 |
| DI | GATATT | 3301.40 | 3271 | 0.991 | −0.009 |
| DI | GATATA | 1518.36 | 1495 | 0.985 | −0.016 |
| DI | GACATA | 1715.16 | 1565 | 0.912 | −0.092 |
| DI | GATATC | 4174.83 | 2205 | 0.528 | −0.638 |
| DK | GACAAG | 5562.52 | 7324 | 1.317 | 0.275 |
| DK | GACAAA | 4295.02 | 4794 | 1.116 | 0.110 |
| DK | GATAAA | 3802.20 | 3855 | 1.014 | 0.014 |
| DK | GATAAG | 4924.27 | 2611 | 0.530 | −0.634 |
| DL | GACCTC | 3785.97 | 5029 | 1.328 | 0.284 |
| DL | GACTTG | 2557.95 | 3396 | 1.328 | 0.283 |
| DL | GATTTA | 1347.95 | 1740 | 1.291 | 0.255 |
| DL | GACCTG | 7867.71 | 9796 | 1.245 | 0.219 |
| DL | GATTTG | 2264.44 | 2687 | 1.187 | 0.171 |
| DL | GACCTT | 2610.58 | 2774 | 1.063 | 0.061 |
| DL | GATCTT | 2311.04 | 2416 | 1.045 | 0.044 |
| DL | GACCTA | 1407.87 | 1416 | 1.006 | 0.006 |
| DL | GACTTA | 1522.66 | 1403 | 0.921 | −0.082 |
| DL | GATCTA | 1246.33 | 1020 | 0.818 | −0.200 |
| DL | GATCTC | 3351.56 | 2214 | 0.661 | −0.415 |
| DL | GATCTG | 6964.95 | 3348 | 0.481 | −0.733 |
| DM | GACATG | 4089.63 | 5411 | 1.323 | 0.280 |
| DM | GATATG | 3620.37 | 2299 | 0.635 | −0.454 |
| DN | GACAAC | 3511.00 | 4849 | 1.381 | 0.323 |
| DN | GACAAT | 3187.82 | 3349 | 1.051 | 0.049 |
| DN | GATAAT | 2822.05 | 2549 | 0.903 | −0.102 |
| DN | GATAAC | 3108.14 | 1882 | 0.606 | −0.502 |
| DP | GACCCC | 3732.11 | 5119 | 1.372 | 0.316 |
| DP | GACCCG | 1352.28 | 1692 | 1.251 | 0.224 |
| DP | GACCCT | 3342.62 | 3700 | 1.107 | 0.102 |
| DP | GATCCT | 2959.08 | 3111 | 1.051 | 0.050 |
| DP | GACCCA | 3226.05 | 3205 | 0.993 | −0.007 |
| DP | GATCCA | 2855.89 | 2349 | 0.823 | −0.195 |
| DP | GATCCC | 3303.88 | 2338 | 0.708 | −0.346 |
| DP | GATCCG | 1197.11 | 455 | 0.380 | −0.967 |
| DQ | GACCAG | 5250.37 | 6524 | 1.243 | 0.217 |
| DQ | GACCAA | 1880.22 | 2169 | 1.154 | 0.143 |
| DQ | GATCAA | 1664.48 | 1808 | 1.086 | 0.083 |
| DQ | GATCAG | 4647.93 | 2942 | 0.633 | −0.457 |
| DR | GACCGC | 1807.77 | 2634 | 1.457 | 0.376 |
| DR | GACAGA | 1994.00 | 2869 | 1.439 | 0.364 |
| DR | GACAGG | 1957.57 | 2730 | 1.395 | 0.333 |
| DR | GACCGT | 773.97 | 1029 | 1.330 | 0.285 |
| DR | GACCGG | 1977.16 | 2568 | 1.299 | 0.261 |
| DR | GACCGA | 1071.78 | 1292 | 1.205 | 0.187 |
| DR | GATCGA | 948.80 | 923 | 0.973 | −0.028 |
| DR | GATCGT | 685.16 | 626 | 0.914 | −0.090 |
| DR | GATAGA | 1765.20 | 1123 | 0.636 | −0.452 |
| DR | GATCGG | 1750.30 | 859 | 0.491 | −0.712 |
| DR | GATCGC | 1600.34 | 754 | 0.471 | −0.753 |
| DR | GATAGG | 1732.96 | 658 | 0.380 | −0.968 |
| DS | GACTCG | 918.57 | 1527 | 1.662 | 0.508 |
| DS | GACAGC | 3935.48 | 6143 | 1.561 | 0.445 |
| DS | GACAGT | 2482.92 | 3657 | 1.473 | 0.387 |
| DS | GATTCT | 2675.01 | 2968 | 1.110 | 0.104 |
| DS | GACTCC | 3473.65 | 3800 | 1.094 | 0.090 |
| DS | GATTCA | 2162.59 | 2129 | 0.984 | −0.016 |
| DS | GACTCA | 2442.89 | 2382 | 0.975 | −0.025 |
| DS | GACTCT | 3021.73 | 2910 | 0.963 | −0.038 |
| DS | GATTCC | 3075.07 | 2186 | 0.711 | −0.341 |
| DS | GATAGT | 2198.02 | 1355 | 0.616 | −0.484 |
| DS | GATTCG | 813.17 | 414 | 0.509 | −0.675 |
| DS | GATAGC | 3483.91 | 1212 | 0.348 | −1.056 |
| DT | GACACG | 1110.58 | 1842 | 1.659 | 0.506 |
| DT | GACACC | 3380.79 | 4666 | 1.380 | 0.322 |
| DT | GACACA | 2736.88 | 3538 | 1.293 | 0.257 |
| DT | GACACT | 2420.30 | 2688 | 1.111 | 0.105 |
| DT | GATACT | 2142.59 | 1731 | 0.808 | −0.213 |
| DT | GATACA | 2422.85 | 1788 | 0.738 | −0.304 |
| DT | GATACC | 2992.87 | 1586 | 0.530 | −0.635 |
| DT | GATACG | 983.15 | 351 | 0.357 | −1.030 |
| DV | GATGTT | 1957.96 | 3699 | 1.889 | 0.636 |
| DV | GATGTA | 1271.37 | 2214 | 1.741 | 0.555 |
| DV | GATGTC | 2506.81 | 3869 | 1.543 | 0.434 |
| DV | GATGTG | 4959.23 | 6668 | 1.345 | 0.296 |
| DV | GACGTG | 5602.02 | 3616 | 0.645 | −0.438 |
| DV | GACGTC | 2831.73 | 1654 | 0.584 | −0.538 |
| DV | GACGTT | 2211.73 | 672 | 0.304 | −1.191 |
| DV | GACGTA | 1436.16 | 385 | 0.268 | −1.316 |
| DW | GACTGG | 2619.27 | 3853 | 1.471 | 0.386 |
| DW | GATTGG | 2318.73 | 1085 | 0.468 | −0.759 |
| DY | GACTAC | 3307.71 | 3930 | 1.188 | 0.172 |
| DY | GATTAT | 2379.36 | 2608 | 1.096 | 0.092 |
| DY | GACTAT | 2687.76 | 2853 | 1.061 | 0.060 |
| DY | GATTAC | 2928.18 | 1912 | 0.653 | −0.426 |
| EA | GAGGCG | 2437.29 | 3179 | 1.304 | 0.266 |
| EA | GAAGCA | 3880.59 | 4844 | 1.248 | 0.222 |
| EA | GAAGCT | 4432.94 | 5143 | 1.160 | 0.149 |
| EA | GAGGCC | 9014.27 | 9805 | 1.088 | 0.084 |
| EA | GAGGCT | 5928.25 | 5314 | 0.896 | −0.109 |
| EA | GAGGCA | 5189.57 | 4530 | 0.873 | −0.136 |
| EA | GAAGCC | 6740.57 | 5649 | 0.838 | −0.177 |
| EA | GAAGCG | 1822.52 | 982 | 0.539 | −0.618 |
| EC | GAATGT | 2182.58 | 3541 | 1.622 | 0.484 |
| EC | GAGTGT | 2918.80 | 2792 | 0.957 | −0.044 |
| EC | GAGTGC | 3465.35 | 2987 | 0.862 | −0.149 |
| EC | GAATGC | 2591.27 | 1838 | 0.709 | −0.343 |
| ED | GAAGAT | 6605.82 | 9691 | 1.467 | 0.383 |
| ED | GAGGAC | 9979.09 | 9684 | 0.970 | −0.030 |
| ED | GAAGAC | 7462.02 | 6820 | 0.914 | −0.090 |
| ED | GAGGAT | 8834.07 | 6686 | 0.757 | −0.279 |
| EE | GAAGAA | 10747.11 | 14461 | 1.346 | 0.297 |
| EE | GAGGAG | 19220.31 | 21731 | 1.131 | 0.123 |
| EE | GAAGAG | 14372.29 | 11875 | 0.826 | −0.191 |
| EE | GAGGAA | 14372.29 | 10645 | 0.741 | −0.300 |
| EF | GAATTT | 3136.91 | 4237 | 1.351 | 0.301 |
| EF | GAGTTC | 4801.58 | 4739 | 0.987 | −0.013 |
| EF | GAGTTT | 4195.05 | 4095 | 0.976 | −0.024 |
| EF | GAATTC | 3590.46 | 2653 | 0.739 | −0.303 |
| EG | GAAGGA | 3358.73 | 5032 | 1.498 | 0.404 |
| EG | GAAGGT | 2174.51 | 2839 | 1.306 | 0.267 |
| EG | GAAGGG | 3278.97 | 3559 | 1.085 | 0.082 |
| EG | GAGGGC | 6090.10 | 6505 | 1.068 | 0.066 |
| EG | GAAGGC | 4553.97 | 4340 | 0.953 | −0.048 |
| EG | GAGGGG | 4385.02 | 3795 | 0.865 | −0.145 |
| EG | GAGGGT | 2908.01 | 2378 | 0.818 | −0.201 |
| EG | GAGGGA | 4491.69 | 2793 | 0.622 | −0.475 |
| EH | GAACAT | 2017.28 | 2539 | 1.259 | 0.230 |
| EH | GAGCAC | 3720.16 | 4190 | 1.126 | 0.119 |
| EH | GAGCAT | 2697.74 | 2448 | 0.907 | −0.097 |
| EH | GAACAC | 2781.81 | 2040 | 0.733 | −0.310 |
| EI | GAAATA | 1687.78 | 3007 | 1.782 | 0.578 |
| EI | GAAATT | 3669.78 | 4788 | 1.305 | 0.266 |
| EI | GAGATC | 6206.03 | 6191 | 0.998 | −0.002 |
| EI | GAGATT | 4907.66 | 3978 | 0.811 | −0.210 |
| EI | GAGATA | 2257.09 | 1785 | 0.791 | −0.235 |
| EI | GAAATC | 4640.66 | 3620 | 0.780 | −0.248 |
| EK | GAGAAG | 12729.57 | 15133 | 1.189 | 0.173 |
| EK | GAAAAA | 7349.75 | 7522 | 1.023 | 0.023 |
| EK | GAGAAA | 9828.94 | 9127 | 0.929 | −0.074 |
| EK | GAAAAG | 9518.74 | 7645 | 0.803 | −0.219 |
| EL | GAGCTG | 10945.64 | 15625 | 1.428 | 0.356 |
| EL | GAATTA | 1584.03 | 2256 | 1.424 | 0.354 |
| EL | GAACTA | 1464.61 | 1830 | 1.249 | 0.223 |
| EL | GAACTT | 2715.79 | 3371 | 1.241 | 0.216 |
| EL | GAGCTC | 5267.08 | 5877 | 1.116 | 0.110 |
| EL | GAGCTA | 1958.64 | 2049 | 1.046 | 0.045 |
| EL | GAATTG | 2661.03 | 2335 | 0.877 | −0.131 |
| EL | GAGCTT | 3631.87 | 3084 | 0.849 | −0.164 |
| EL | GAGTTG | 3558.64 | 2719 | 0.764 | −0.269 |
| EL | GAACTC | 3938.54 | 2632 | 0.668 | −0.403 |
| EL | GAGTTA | 2118.35 | 1357 | 0.641 | −0.445 |
| EL | GAACTG | 8184.78 | 4894 | 0.598 | −0.514 |
| EM | GAAATG | 4983.92 | 5010 | 1.005 | 0.005 |
| EM | GAGATG | 6665.08 | 6639 | 0.996 | −0.004 |
| EN | GAAAAT | 4791.73 | 6977 | 1.456 | 0.376 |
| EN | GAGAAC | 7057.70 | 6756 | 0.957 | −0.044 |
| EN | GAAAAC | 5277.51 | 4930 | 0.934 | −0.068 |
| EN | GAGAAT | 6408.07 | 4872 | 0.760 | −0.274 |
| EP | GAGCCG | 1650.94 | 2438 | 1.477 | 0.390 |
| EP | GAGCCC | 4556.38 | 6270 | 1.376 | 0.319 |
| EP | GAGCCT | 4080.86 | 4236 | 1.038 | 0.037 |
| EP | GAGCCA | 3938.55 | 4067 | 1.033 | 0.032 |
| EP | GAACCA | 2945.12 | 2684 | 0.911 | −0.093 |
| EP | GAACCT | 3051.53 | 2547 | 0.835 | −0.181 |
| EP | GAACCC | 3407.10 | 2106 | 0.618 | −0.481 |
| EP | GAACCG | 1234.52 | 517 | 0.419 | −0.870 |
| EQ | GAACAA | 2579.50 | 3396 | 1.317 | 0.275 |
| EQ | GAGCAG | 9632.80 | 11185 | 1.161 | 0.149 |
| EQ | GAGCAA | 3449.61 | 3185 | 0.923 | −0.080 |
| EQ | GAACAG | 7203.08 | 5099 | 0.708 | −0.345 |
| ER | GAAAGA | 2650.27 | 3769 | 1.422 | 0.352 |
| ER | GAGAGG | 3479.50 | 4315 | 1.240 | 0.215 |
| ER | GAGCGG | 3514.32 | 4356 | 1.240 | 0.215 |
| ER | GAGCGC | 3213.23 | 3682 | 1.146 | 0.136 |
| ER | GAAAGG | 2601.85 | 2679 | 1.030 | 0.029 |
| ER | GAGAGA | 3544.25 | 3633 | 1.025 | 0.025 |
| ER | GAGCGT | 1375.70 | 1286 | 0.935 | −0.067 |
| ER | GAACGT | 1028.70 | 894 | 0.869 | −0.140 |
| ER | GAACGA | 1424.52 | 1188 | 0.834 | −0.182 |
| ER | GAGCGA | 1905.04 | 1562 | 0.820 | −0.199 |
| ER | GAACGG | 2627.88 | 1333 | 0.507 | −0.679 |
| ER | GAACGC | 2402.74 | 1071 | 0.446 | −0.808 |
| ES | GAAAGT | 2081.93 | 3138 | 1.507 | 0.410 |
| ES | GAGAGC | 4413.03 | 5786 | 1.311 | 0.271 |
| ES | GAGAGT | 2784.21 | 3237 | 1.163 | 0.151 |
| ES | GAGTCG | 1030.03 | 1174 | 1.140 | 0.131 |
| ES | GAATCT | 2533.73 | 2812 | 1.110 | 0.104 |
| ES | GAATCA | 2048.37 | 2131 | 1.040 | 0.040 |
| ES | GAAAGC | 3299.91 | 2880 | 0.873 | −0.136 |
| ES | GAGTCC | 3895.16 | 3392 | 0.871 | −0.138 |
| ES | GAGTCT | 3388.40 | 2799 | 0.826 | −0.191 |
| ES | GAGTCA | 2739.33 | 2198 | 0.802 | −0.220 |
| ES | GAATCC | 2912.67 | 1943 | 0.667 | −0.405 |
| ES | GAATCG | 770.22 | 407 | 0.528 | −0.638 |
| ET | GAGACG | 1658.42 | 2190 | 1.321 | 0.278 |
| ET | GAAACA | 3056.09 | 3851 | 1.260 | 0.231 |
| ET | GAAACT | 2702.59 | 3224 | 1.193 | 0.176 |
| ET | GAGACC | 5048.51 | 5514 | 1.092 | 0.088 |
| ET | GAGACA | 4086.97 | 3619 | 0.885 | −0.122 |
| ET | GAGACT | 3614.21 | 3028 | 0.838 | −0.177 |
| ET | GAAACC | 3775.11 | 2950 | 0.781 | −0.247 |
| ET | GAAACG | 1240.11 | 806 | 0.650 | −0.431 |
| EV | GAAGTA | 1580.16 | 2675 | 1.693 | 0.526 |
| EV | GAAGTT | 2433.50 | 3724 | 1.530 | 0.425 |
| EV | GAGGTG | 8242.83 | 9074 | 1.101 | 0.096 |
| EV | GAAGTC | 3115.66 | 2860 | 0.918 | −0.086 |
| EV | GAGGTC | 4166.62 | 3741 | 0.898 | −0.108 |
| EV | GAAGTG | 6163.71 | 5122 | 0.831 | −0.185 |
| EV | GAGGTT | 3254.36 | 2359 | 0.725 | −0.322 |
| EV | GAGGTA | 2113.17 | 1515 | 0.717 | −0.333 |
| EW | GAGTGG | 3085.08 | 3238 | 1.050 | 0.048 |
| EW | GAATGG | 2306.92 | 2154 | 0.934 | −0.069 |
| EY | GAATAT | 2307.55 | 3428 | 1.486 | 0.396 |
| EY | GAGTAC | 3797.72 | 3796 | 1.000 | 0.000 |
| EY | GAGTAT | 3085.93 | 2596 | 0.841 | −0.173 |
| EY | GAATAC | 2839.80 | 2211 | 0.779 | −0.250 |
| FA | TTTGCA | 1643.98 | 3299 | 2.007 | 0.696 |
| FA | TTTGCT | 1877.98 | 3746 | 1.995 | 0.690 |
| FA | TTTGCC | 2855.59 | 4348 | 1.523 | 0.420 |
| FA | TTTGCG | 772.10 | 622 | 0.806 | −0.216 |
| FA | TTCGCG | 883.73 | 598 | 0.677 | −0.391 |
| FA | TTCGCC | 3268.46 | 1802 | 0.551 | −0.595 |
| FA | TTCGCT | 2149.50 | 516 | 0.240 | −1.427 |
| FA | TTCGCA | 1881.67 | 402 | 0.214 | −1.543 |
| FC | TTCTGC | 2058.60 | 3045 | 1.479 | 0.391 |
| FC | TTCTGT | 1733.93 | 2055 | 1.185 | 0.170 |
| FC | TTTTGT | 1514.90 | 1159 | 0.765 | −0.268 |
| FC | TTTTGC | 1798.56 | 847 | 0.471 | −0.753 |
| FD | TTTGAT | 2786.65 | 5380 | 1.931 | 0.658 |
| FD | TTTGAC | 3147.84 | 4737 | 1.505 | 0.409 |
| FD | TTCGAC | 3602.96 | 1746 | 0.485 | −0.724 |
| FD | TTCGAT | 3189.55 | 864 | 0.271 | −1.306 |
| FE | TTTGAA | 3016.02 | 6247 | 2.071 | 0.728 |
| FE | TTTGAG | 4033.37 | 6066 | 1.504 | 0.408 |
| FE | TTCGAG | 4616.53 | 2165 | 0.469 | −0.757 |
| FE | TTCGAA | 3452.08 | 640 | 0.185 | −1.685 |
| FF | TTCTTC | 3429.53 | 5168 | 1.507 | 0.410 |
| FF | TTCTTT | 2996.32 | 2989 | 0.998 | −0.002 |
| FF | TTTTTT | 2617.83 | 1937 | 0.740 | −0.301 |
| FF | TTTTTC | 2996.32 | 1946 | 0.649 | −0.432 |
| FG | TTTGGA | 2068.21 | 4271 | 2.065 | 0.725 |
| FG | TTTGGT | 1339.00 | 2552 | 1.906 | 0.645 |
| FG | TTTGGG | 2019.09 | 3449 | 1.708 | 0.535 |
| FG | TTTGGC | 2804.20 | 3462 | 1.235 | 0.211 |
| FG | TTCGGG | 2311.02 | 1292 | 0.559 | −0.581 |
| FG | TTCGGC | 3209.64 | 1648 | 0.513 | −0.667 |
| FG | TTCGGT | 1532.60 | 419 | 0.273 | −1.297 |
| FG | TTCGGA | 2367.24 | 558 | 0.236 | −1.445 |
| FH | TTCCAC | 2463.48 | 3200 | 1.299 | 0.262 |
| FH | TTTCAT | 1560.78 | 1697 | 1.087 | 0.084 |
| FH | TTCCAT | 1786.44 | 1866 | 1.045 | 0.044 |
| FH | TTTCAC | 2152.30 | 1200 | 0.558 | −0.584 |
| FI | TTCATC | 3454.46 | 5156 | 1.493 | 0.400 |
| FI | TTCATT | 2731.75 | 2953 | 1.081 | 0.078 |
| FI | TTTATT | 2386.67 | 2296 | 0.962 | −0.039 |
| FI | TTTATA | 1097.66 | 950 | 0.865 | −0.144 |
| FI | TTCATA | 1256.36 | 1035 | 0.824 | −0.194 |
| FI | TTTATC | 3018.10 | 1555 | 0.515 | −0.663 |
| FK | TTCAAG | 4090.45 | 5137 | 1.256 | 0.228 |
| FK | TTCAAA | 3158.38 | 3245 | 1.027 | 0.027 |
| FK | TTTAAA | 2759.42 | 2762 | 1.001 | 0.001 |
| FK | TTTAAG | 3573.75 | 2438 | 0.682 | −0.382 |
| FL | TTCCTC | 3228.53 | 4426 | 1.371 | 0.315 |
| FL | TTCCTG | 6709.28 | 8734 | 1.302 | 0.264 |
| FL | TTTTTA | 1134.45 | 1334 | 1.176 | 0.162 |
| FL | TTTCTT | 1945.00 | 2267 | 1.166 | 0.153 |
| FL | TTCCTA | 1200.58 | 1280 | 1.066 | 0.064 |
| FL | TTTCTA | 1048.92 | 1087 | 1.036 | 0.036 |
| FL | TTCTTG | 2181.32 | 2239 | 1.026 | 0.026 |
| FL | TTCCTT | 2226.21 | 2150 | 0.966 | −0.035 |
| FL | TTTTTG | 1905.78 | 1799 | 0.944 | −0.058 |
| FL | TTCTTA | 1298.47 | 1144 | 0.881 | −0.127 |
| FL | TTTCTC | 2820.70 | 1904 | 0.675 | −0.393 |
| FL | TTTCTG | 5861.77 | 3197 | 0.545 | −0.606 |
| FM | TTCATG | 2804.11 | 3662 | 1.306 | 0.267 |
| FM | TTTATG | 2449.89 | 1592 | 0.650 | −0.431 |
| FN | TTCAAC | 2855.47 | 3919 | 1.372 | 0.317 |
| FN | TTTAAT | 2265.13 | 2185 | 0.965 | −0.036 |
| FN | TTCAAT | 2592.63 | 2456 | 0.947 | −0.054 |
| FN | TTTAAC | 2494.77 | 1648 | 0.661 | −0.415 |
| FP | TTCCCG | 961.40 | 1205 | 1.253 | 0.226 |
| FP | TTTCCT | 2076.25 | 2539 | 1.223 | 0.201 |
| FP | TTCCCC | 2653.35 | 3099 | 1.168 | 0.155 |
| FP | TTTCCA | 2003.85 | 2141 | 1.068 | 0.066 |
| FP | TTCCCA | 2293.57 | 2310 | 1.007 | 0.007 |
| FP | TTCCCT | 2376.44 | 2379 | 1.001 | 0.001 |
| FP | TTTCCC | 2318.18 | 1529 | 0.660 | −0.416 |
| FP | TTTCCG | 839.96 | 321 | 0.382 | −0.962 |
| FQ | TTCCAG | 5468.69 | 7069 | 1.293 | 0.257 |
| FQ | TTTCAA | 1711.02 | 1803 | 1.054 | 0.052 |
| FQ | TTCCAA | 1958.40 | 1980 | 1.011 | 0.011 |
| FQ | TTTCAG | 4777.89 | 3064 | 0.641 | −0.444 |
| FR | TTCCGC | 1531.47 | 2588 | 1.690 | 0.525 |
| FR | TTCCGA | 907.97 | 1410 | 1.553 | 0.440 |
| FR | TTCCGG | 1674.97 | 2451 | 1.463 | 0.381 |
| FR | TTCCGT | 655.68 | 893 | 1.362 | 0.309 |
| FR | TTCAGA | 1689.24 | 1852 | 1.096 | 0.092 |
| FR | TTCAGG | 1658.38 | 1810 | 1.091 | 0.087 |
| FR | TTTCGA | 793.28 | 850 | 1.072 | 0.069 |
| FR | TTTCGT | 572.85 | 490 | 0.855 | −0.156 |
| FR | TTTAGA | 1475.86 | 947 | 0.642 | −0.444 |
| FR | TTTAGG | 1448.90 | 691 | 0.477 | −0.740 |
| FR | TTTCGG | 1463.39 | 688 | 0.470 | −0.755 |
| FR | TTTCGC | 1338.02 | 540 | 0.404 | −0.907 |
| FS | TTCTCC | 2990.83 | 4507 | 1.507 | 0.410 |
| FS | TTCAGC | 3388.47 | 4577 | 1.351 | 0.301 |
| FS | TTCAGT | 2137.80 | 2692 | 1.259 | 0.231 |
| FS | TTCTCG | 790.89 | 910 | 1.151 | 0.140 |
| FS | TTTTCT | 2273.08 | 2536 | 1.116 | 0.109 |
| FS | TTCTCT | 2601.73 | 2741 | 1.054 | 0.052 |
| FS | TTTTCA | 1837.65 | 1903 | 1.036 | 0.035 |
| FS | TTCTCA | 2103.34 | 1997 | 0.949 | −0.052 |
| FS | TTTTCC | 2613.03 | 1872 | 0.716 | −0.334 |
| FS | TTTAGT | 1867.76 | 1201 | 0.643 | −0.442 |
| FS | TTTTCG | 690.99 | 258 | 0.373 | −0.985 |
| FS | TTTAGC | 2960.44 | 1062 | 0.359 | −1.025 |
| FT | TTCACC | 2909.29 | 4513 | 1.551 | 0.439 |
| FT | TTCACG | 955.69 | 1315 | 1.376 | 0.319 |
| FT | TTCACT | 2082.75 | 2494 | 1.197 | 0.180 |
| FT | TTCACA | 2355.18 | 2372 | 1.007 | 0.007 |
| FT | TTTACT | 1819.66 | 1622 | 0.891 | −0.115 |
| FT | TTTACA | 2057.68 | 1485 | 0.722 | −0.326 |
| FT | TTTACC | 2541.79 | 1495 | 0.588 | −0.531 |
| FT | TTTACG | 834.97 | 261 | 0.313 | −1.163 |
| FV | TTTGTA | 912.19 | 1711 | 1.876 | 0.629 |
| FV | TTTGTT | 1404.80 | 2620 | 1.865 | 0.623 |
| FV | TTTGTC | 1798.60 | 2635 | 1.465 | 0.382 |
| FV | TTTGTG | 3558.17 | 5206 | 1.463 | 0.381 |
| FV | TTCGTG | 4072.62 | 2589 | 0.636 | −0.453 |
| FV | TTCGTC | 2058.64 | 1086 | 0.528 | −0.640 |
| FV | TTCGTT | 1607.91 | 386 | 0.240 | −1.427 |
| FV | TTCGTA | 1044.07 | 224 | 0.215 | −1.539 |
| FW | TTCTGG | 2126.30 | 2834 | 1.333 | 0.287 |
| FW | TTTTGG | 1857.70 | 1150 | 0.619 | −0.480 |
| FY | TTCTAC | 2720.70 | 3710 | 1.364 | 0.310 |
| FY | TTTTAT | 1931.51 | 2003 | 1.037 | 0.036 |
| FY | TTCTAT | 2210.77 | 2145 | 0.970 | −0.030 |
| FY | TTTTAC | 2377.02 | 1382 | 0.581 | −0.542 |
| GA | GGTGCT | 1531.20 | 2505 | 1.636 | 0.492 |
| GA | GGGGCG | 949.27 | 1433 | 1.510 | 0.412 |
| GA | GGGGCC | 3510.85 | 5061 | 1.442 | 0.366 |
| GA | GGTGCC | 2328.29 | 3109 | 1.335 | 0.289 |
| GA | GGAGCA | 2070.38 | 2678 | 1.293 | 0.257 |
| GA | GGTGCA | 1340.41 | 1715 | 1.279 | 0.246 |
| GA | GGCGCG | 1318.38 | 1659 | 1.258 | 0.230 |
| GA | GGAGCT | 2365.08 | 2975 | 1.258 | 0.229 |
| GA | GGGGCT | 2308.91 | 2850 | 1.234 | 0.211 |
| GA | GGAGCC | 3596.25 | 3845 | 1.069 | 0.067 |
| GA | GGGGCA | 2021.22 | 2074 | 1.026 | 0.026 |
| GA | GGTGCG | 629.52 | 501 | 0.796 | −0.228 |
| GA | GGAGCG | 972.36 | 712 | 0.732 | −0.312 |
| GA | GGCGCC | 4876.02 | 3121 | 0.640 | −0.446 |
| GA | GGCGCT | 3206.72 | 906 | 0.283 | −1.264 |
| GA | GGCGCA | 2807.15 | 688 | 0.245 | −1.406 |
| GC | GGCTGC | 1888.96 | 4102 | 2.172 | 0.775 |
| GC | GGCTGT | 1591.04 | 2360 | 1.483 | 0.394 |
| GC | GGTTGT | 759.72 | 658 | 0.866 | −0.144 |
| GC | GGATGT | 1173.45 | 793 | 0.676 | −0.392 |
| GC | GGTTGC | 901.97 | 523 | 0.580 | −0.545 |
| GC | GGATGC | 1393.18 | 655 | 0.470 | −0.755 |
| GC | GGGTGC | 1360.09 | 628 | 0.462 | −0.773 |
| GC | GGGTGT | 1145.59 | 495 | 0.432 | −0.839 |
| GD | GGGGAC | 3126.50 | 4967 | 1.589 | 0.463 |
| GD | GGTGAT | 1835.49 | 2621 | 1.428 | 0.356 |
| GD | GGTGAC | 2073.40 | 2960 | 1.428 | 0.356 |
| GD | GGAGAT | 2835.09 | 3829 | 1.351 | 0.301 |
| GD | GGAGAC | 3202.56 | 4240 | 1.324 | 0.281 |
| GD | GGGGAT | 2767.76 | 2575 | 0.930 | −0.072 |
| GD | GGCGAC | 4342.22 | 1955 | 0.450 | −0.798 |
| GD | GGCGAT | 3843.98 | 880 | 0.229 | −1.474 |
| GE | GGAGAA | 3433.99 | 5903 | 1.719 | 0.542 |
| GE | GGGGAG | 4483.27 | 6552 | 1.461 | 0.379 |
| GE | GGTGAA | 2223.23 | 3248 | 1.461 | 0.379 |
| GE | GGAGAG | 4592.33 | 5961 | 1.298 | 0.261 |
| GE | GGTGAG | 2973.17 | 2988 | 1.005 | 0.005 |
| GE | GGGGAA | 3352.44 | 3041 | 0.907 | −0.098 |
| GE | GGCGAG | 6226.56 | 3530 | 0.567 | −0.568 |
| GE | GGCGAA | 4656.01 | 718 | 0.154 | −1.869 |
| GF | GGCTTC | 3466.22 | 6121 | 1.766 | 0.569 |
| GF | GGATTT | 2233.54 | 2666 | 1.194 | 0.177 |
| GF | GGTTTT | 1446.04 | 1665 | 1.151 | 0.141 |
| GF | GGCTTT | 3028.37 | 3201 | 1.057 | 0.055 |
| GF | GGTTTC | 1655.11 | 1548 | 0.935 | −0.067 |
| GF | GGATTC | 2556.47 | 1534 | 0.600 | −0.511 |
| GF | GGGTTT | 2180.50 | 1244 | 0.571 | −0.561 |
| GF | GGGTTC | 2495.76 | 1083 | 0.434 | −0.835 |
| GG | GGTGGT | 1061.28 | 2286 | 2.154 | 0.767 |
| GG | GGTGGC | 2222.59 | 3657 | 1.645 | 0.498 |
| GG | GGTGGA | 1639.25 | 2618 | 1.597 | 0.468 |
| GG | GGAGGA | 2531.97 | 3609 | 1.425 | 0.354 |
| GG | GGTGGG | 1600.32 | 2267 | 1.417 | 0.348 |
| GG | GGGGGC | 3351.47 | 4673 | 1.394 | 0.332 |
| GG | GGAGGT | 1639.25 | 2152 | 1.313 | 0.272 |
| GG | GGAGGC | 3433.00 | 3776 | 1.100 | 0.095 |
| GG | GGCGGC | 4654.67 | 4787 | 1.028 | 0.028 |
| GG | GGGGGT | 1600.32 | 1543 | 0.964 | −0.036 |
| GG | GGAGGG | 2471.84 | 2351 | 0.951 | −0.050 |
| GG | GGGGGA | 2471.84 | 1517 | 0.614 | −0.488 |
| GG | GGCGGG | 3351.47 | 2001 | 0.597 | −0.516 |
| GG | GGGGGG | 2413.14 | 1080 | 0.448 | −0.804 |
| GG | GGCGGT | 2222.59 | 936 | 0.421 | −0.865 |
| GG | GGCGGA | 3433.00 | 845 | 0.246 | −1.402 |
| GH | GGCCAC | 2540.15 | 3679 | 1.448 | 0.370 |
| GH | GGTCAT | 879.57 | 1022 | 1.162 | 0.150 |
| GH | GGACAT | 1358.57 | 1438 | 1.058 | 0.057 |
| GH | GGCCAT | 1842.04 | 1679 | 0.911 | −0.093 |
| GH | GGGCAC | 1828.97 | 1629 | 0.891 | −0.116 |
| GH | GGTCAC | 1212.92 | 1008 | 0.831 | −0.185 |
| GH | GGACAC | 1873.46 | 1479 | 0.789 | −0.236 |
| GH | GGGCAT | 1326.31 | 928 | 0.700 | −0.357 |
| GI | GGCATC | 3372.48 | 5474 | 1.623 | 0.484 |
| GI | GGAATA | 904.63 | 1338 | 1.479 | 0.391 |
| GI | GGAATT | 1966.96 | 2560 | 1.302 | 0.264 |
| GI | GGCATT | 2666.92 | 2670 | 1.001 | 0.001 |
| GI | GGTATT | 1273.45 | 1052 | 0.826 | −0.191 |
| GI | GGGATC | 2428.27 | 1958 | 0.806 | −0.215 |
| GI | GGTATA | 585.67 | 461 | 0.787 | −0.239 |
| GI | GGAATC | 2487.34 | 1910 | 0.768 | −0.264 |
| GI | GGGATA | 883.14 | 666 | 0.754 | −0.282 |
| GI | GGGATT | 1920.24 | 1421 | 0.740 | −0.301 |
| GI | GGCATA | 1226.55 | 885 | 0.722 | −0.326 |
| GI | GGTATC | 1610.35 | 931 | 0.578 | −0.548 |
| GK | GGAAAA | 3199.11 | 4553 | 1.423 | 0.353 |
| GK | GGGAAG | 4044.81 | 5674 | 1.403 | 0.338 |
| GK | GGGAAA | 3123.14 | 4119 | 1.319 | 0.277 |
| GK | GGCAAG | 5617.61 | 5712 | 1.017 | 0.017 |
| GK | GGAAAG | 4143.21 | 3706 | 0.894 | −0.112 |
| GK | GGCAAA | 4337.55 | 3581 | 0.826 | −0.192 |
| GK | GGTAAA | 2071.17 | 1334 | 0.644 | −0.440 |
| GK | GGTAAG | 2682.40 | 540 | 0.201 | −1.603 |
| GL | GGCCTC | 3017.19 | 4559 | 1.511 | 0.413 |
| GL | GGTTTA | 579.43 | 820 | 1.415 | 0.347 |
| GL | GGTTTG | 973.39 | 1294 | 1.329 | 0.285 |
| GL | GGGCTG | 4514.62 | 5878 | 1.302 | 0.264 |
| GL | GGTCTT | 993.42 | 1258 | 1.266 | 0.236 |
| GL | GGCCTG | 6270.10 | 7822 | 1.248 | 0.221 |
| GL | GGGCTC | 2172.45 | 2563 | 1.180 | 0.165 |
| GL | GGATTA | 894.98 | 991 | 1.107 | 0.102 |
| GL | GGACTT | 1534.44 | 1613 | 1.051 | 0.050 |
| GL | GGCTTG | 2038.53 | 2109 | 1.035 | 0.034 |
| GL | GGCCTT | 2080.48 | 2098 | 1.008 | 0.008 |
| GL | GGACTA | 827.51 | 799 | 0.966 | −0.035 |
| GL | GGGCTT | 1497.99 | 1445 | 0.965 | −0.036 |
| GL | GGTCTC | 1440.70 | 1365 | 0.947 | −0.054 |
| GL | GGTCTA | 535.75 | 487 | 0.909 | −0.095 |
| GL | GGGCTA | 807.86 | 726 | 0.899 | −0.107 |
| GL | GGCCTA | 1121.99 | 968 | 0.863 | −0.148 |
| GL | GGCTTA | 1213.47 | 935 | 0.771 | −0.261 |
| GL | GGACTC | 2225.29 | 1656 | 0.744 | −0.295 |
| GL | GGATTG | 1503.50 | 1062 | 0.706 | −0.348 |
| GL | GGTCTG | 2993.96 | 2034 | 0.679 | −0.387 |
| GL | GGGTTG | 1467.79 | 870 | 0.593 | −0.523 |
| GL | GGGTTA | 873.73 | 467 | 0.534 | −0.626 |
| GL | GGACTG | 4624.44 | 2384 | 0.516 | −0.663 |
| GM | GGCATG | 3177.11 | 3953 | 1.244 | 0.219 |
| GM | GGAATG | 2343.24 | 2482 | 1.059 | 0.058 |
| GM | GGGATG | 2287.59 | 2247 | 0.982 | −0.018 |
| GM | GGTATG | 1517.06 | 643 | 0.424 | −0.858 |
| GN | GGAAAT | 2150.19 | 3332 | 1.550 | 0.438 |
| GN | GGGAAC | 2311.93 | 2816 | 1.218 | 0.197 |
| GN | GGCAAC | 3210.92 | 3701 | 1.153 | 0.142 |
| GN | GGAAAC | 2368.18 | 2679 | 1.131 | 0.123 |
| GN | GGGAAT | 2099.13 | 1823 | 0.868 | −0.141 |
| GN | GGCAAT | 2915.36 | 2061 | 0.707 | −0.347 |
| GN | GGTAAT | 1392.08 | 784 | 0.563 | −0.574 |
| GN | GGTAAC | 1533.21 | 785 | 0.512 | −0.669 |
| GP | GGGCCC | 2634.22 | 3947 | 1.498 | 0.404 |
| GP | GGGCCG | 954.47 | 1417 | 1.485 | 0.395 |
| GP | GGCCCC | 3658.52 | 4576 | 1.251 | 0.224 |
| GP | GGCCCG | 1325.61 | 1623 | 1.224 | 0.202 |
| GP | GGTCCT | 1564.62 | 1910 | 1.221 | 0.199 |
| GP | GGGCCT | 2359.31 | 2542 | 1.077 | 0.075 |
| GP | GGTCCC | 1746.93 | 1827 | 1.046 | 0.045 |
| GP | GGCCCT | 3276.71 | 2994 | 0.914 | −0.090 |
| GP | GGGCCA | 2277.03 | 2003 | 0.880 | −0.128 |
| GP | GGTCCA | 1510.06 | 1264 | 0.837 | −0.178 |
| GP | GGACCC | 2698.30 | 2240 | 0.830 | −0.186 |
| GP | GGACCA | 2332.42 | 1908 | 0.818 | −0.201 |
| GP | GGACCT | 2416.70 | 1957 | 0.810 | −0.211 |
| GP | GGCCCA | 3162.44 | 2548 | 0.806 | −0.216 |
| GP | GGTCCG | 632.98 | 351 | 0.555 | −0.590 |
| GP | GGACCG | 977.69 | 421 | 0.431 | −0.843 |
| GQ | GGACAA | 1382.58 | 1677 | 1.213 | 0.193 |
| GQ | GGGCAG | 3769.06 | 4425 | 1.174 | 0.160 |
| GQ | GGCCAG | 5234.64 | 6081 | 1.162 | 0.150 |
| GQ | GGTCAA | 895.11 | 953 | 1.065 | 0.063 |
| GQ | GGCCAA | 1874.58 | 1593 | 0.850 | −0.163 |
| GQ | GGGCAA | 1349.74 | 1124 | 0.833 | −0.183 |
| GQ | GGACAG | 3860.75 | 3134 | 0.812 | −0.209 |
| GQ | GGTCAG | 2499.53 | 1879 | 0.752 | −0.285 |
| GR | GGCCGC | 1832.29 | 3615 | 1.973 | 0.680 |
| GR | GGAAGA | 1490.60 | 2294 | 1.539 | 0.431 |
| GR | GGCCGG | 2003.98 | 2892 | 1.443 | 0.367 |
| GR | GGCCGT | 784.47 | 1022 | 1.303 | 0.265 |
| GR | GGTCGT | 374.58 | 450 | 1.201 | 0.183 |
| GR | GGCCGA | 1086.32 | 1252 | 1.153 | 0.142 |
| GR | GGGCGC | 1319.29 | 1471 | 1.115 | 0.109 |
| GR | GGTCGA | 518.71 | 546 | 1.053 | 0.051 |
| GR | GGCAGG | 1984.13 | 2022 | 1.019 | 0.019 |
| GR | GGGAGG | 1428.62 | 1435 | 1.004 | 0.004 |
| GR | GGGCGG | 1442.91 | 1437 | 0.996 | −0.004 |
| GR | GGAAGG | 1463.37 | 1370 | 0.936 | −0.066 |
| GR | GGGAGA | 1455.20 | 1344 | 0.924 | −0.079 |
| GR | GGACGT | 578.58 | 514 | 0.888 | −0.118 |
| GR | GGACGA | 801.20 | 671 | 0.837 | −0.177 |
| GR | GGGCGT | 564.84 | 471 | 0.834 | −0.182 |
| GR | GGCAGA | 2021.05 | 1684 | 0.833 | −0.182 |
| GR | GGGCGA | 782.17 | 626 | 0.800 | −0.223 |
| GR | GGTCGC | 874.92 | 596 | 0.681 | −0.384 |
| GR | GGTCGG | 956.90 | 555 | 0.580 | −0.545 |
| GR | GGTAGA | 965.05 | 529 | 0.548 | −0.601 |
| GR | GGACGC | 1351.39 | 729 | 0.539 | −0.617 |
| GR | GGACGG | 1478.01 | 737 | 0.499 | −0.696 |
| GR | GGTAGG | 947.42 | 244 | 0.258 | −1.357 |
| GS | GGCAGC | 3581.32 | 6542 | 1.827 | 0.603 |
| GS | GGCTCC | 3161.05 | 5376 | 1.701 | 0.531 |
| GS | GGCTCG | 835.91 | 1323 | 1.583 | 0.459 |
| GS | GGCAGT | 2259.47 | 2875 | 1.272 | 0.241 |
| GS | GGAAGT | 1666.45 | 2085 | 1.251 | 0.224 |
| GS | GGTTCT | 1313.02 | 1563 | 1.190 | 0.174 |
| GS | GGCTCT | 2749.80 | 3087 | 1.123 | 0.116 |
| GS | GGGAGC | 2578.63 | 2566 | 0.995 | −0.005 |
| GS | GGTTCC | 1509.39 | 1428 | 0.946 | −0.055 |
| GS | GGCTCA | 2223.05 | 2101 | 0.945 | −0.056 |
| GS | GGTTCA | 1061.50 | 981 | 0.924 | −0.079 |
| GS | GGAAGC | 2641.36 | 2137 | 0.809 | −0.212 |
| GS | GGATCA | 1639.59 | 1281 | 0.781 | −0.247 |
| GS | GGGAGT | 1626.88 | 1267 | 0.779 | −0.250 |
| GS | GGATCT | 2028.08 | 1470 | 0.725 | −0.322 |
| GS | GGGTCC | 2276.03 | 1646 | 0.723 | −0.324 |
| GS | GGGTCT | 1979.92 | 1280 | 0.646 | −0.436 |
| GS | GGGTCG | 601.87 | 379 | 0.630 | −0.463 |
| GS | GGTAGT | 1078.89 | 646 | 0.599 | −0.513 |
| GS | GGATCC | 2331.40 | 1342 | 0.576 | −0.552 |
| GS | GGGTCA | 1600.65 | 887 | 0.554 | −0.590 |
| GS | GGTTCG | 399.14 | 209 | 0.524 | −0.647 |
| GS | GGATCG | 616.51 | 276 | 0.448 | −0.804 |
| GS | GGTAGC | 1710.07 | 723 | 0.423 | −0.861 |
| GT | GGCACC | 3271.07 | 4870 | 1.489 | 0.398 |
| GT | GGCACG | 1074.53 | 1368 | 1.273 | 0.241 |
| GT | GGGACC | 2355.25 | 2817 | 1.196 | 0.179 |
| GT | GGAACA | 1953.05 | 2290 | 1.173 | 0.159 |
| GT | GGAACT | 1727.13 | 1900 | 1.100 | 0.095 |
| GT | GGGACG | 773.69 | 838 | 1.083 | 0.080 |
| GT | GGGACA | 1906.66 | 1903 | 0.998 | −0.002 |
| GT | GGCACT | 2341.75 | 2331 | 0.995 | −0.005 |
| GT | GGCACA | 2648.06 | 2499 | 0.944 | −0.058 |
| GT | GGGACT | 1686.11 | 1534 | 0.910 | −0.095 |
| GT | GGAACC | 2412.54 | 1841 | 0.763 | −0.270 |
| GT | GGTACT | 1118.18 | 840 | 0.751 | −0.286 |
| GT | GGTACC | 1561.93 | 994 | 0.636 | −0.452 |
| GT | GGTACA | 1264.44 | 780 | 0.617 | −0.483 |
| GT | GGAACG | 792.51 | 445 | 0.562 | −0.577 |
| GT | GGTACG | 513.09 | 150 | 0.292 | −1.230 |
| GV | GGTGTT | 816.93 | 1802 | 2.206 | 0.791 |
| GV | GGTGTC | 1045.94 | 2070 | 1.979 | 0.683 |
| GV | GGTGTA | 530.46 | 957 | 1.804 | 0.590 |
| GV | GGTGTG | 2069.18 | 3207 | 1.550 | 0.438 |
| GV | GGAGTA | 819.35 | 1225 | 1.495 | 0.402 |
| GV | GGAGTT | 1261.83 | 1841 | 1.459 | 0.378 |
| GV | GGGGTC | 1577.18 | 2150 | 1.363 | 0.310 |
| GV | GGAGTC | 1615.55 | 1839 | 1.138 | 0.130 |
| GV | GGGGTT | 1231.86 | 1123 | 0.912 | −0.093 |
| GV | GGGGTG | 3120.14 | 2770 | 0.888 | −0.119 |
| GV | GGAGTG | 3196.04 | 2641 | 0.826 | −0.191 |
| GV | GGGGTA | 799.89 | 631 | 0.789 | −0.237 |
| GV | GGCGTC | 2190.46 | 1653 | 0.755 | −0.282 |
| GV | GGCGTG | 4333.39 | 2790 | 0.644 | −0.440 |
| GV | GGCGTT | 1710.87 | 499 | 0.292 | −1.232 |
| GV | GGCGTA | 1110.93 | 232 | 0.209 | −1.566 |
| GW | GGCTGG | 2102.85 | 3748 | 1.782 | 0.578 |
| GW | GGTTGG | 1004.11 | 690 | 0.687 | −0.375 |
| GW | GGATGG | 1550.94 | 1012 | 0.653 | −0.427 |
| GW | GGGTGG | 1514.10 | 722 | 0.477 | −0.741 |
| GY | GGCTAC | 2577.81 | 4581 | 1.777 | 0.575 |
| GY | GGTTAT | 1000.20 | 1309 | 1.309 | 0.269 |
| GY | GGCTAT | 2094.66 | 2528 | 1.207 | 0.188 |
| GY | GGATAT | 1544.90 | 1478 | 0.957 | −0.044 |
| GY | GGTTAC | 1230.90 | 1074 | 0.873 | −0.136 |
| GY | GGATAC | 1901.24 | 1052 | 0.553 | −0.592 |
| GY | GGGTAC | 1856.09 | 982 | 0.529 | −0.637 |
| GY | GGGTAT | 1508.21 | 710 | 0.471 | −0.753 |
| HA | CATGCT | 1101.90 | 1959 | 1.778 | 0.575 |
| HA | CATGCA | 964.61 | 1670 | 1.731 | 0.549 |
| HA | CATGCC | 1675.52 | 2408 | 1.437 | 0.363 |
| HA | CACGCG | 624.72 | 681 | 1.090 | 0.086 |
| HA | CATGCG | 453.03 | 447 | 0.987 | −0.013 |
| HA | CACGCC | 2310.52 | 1649 | 0.714 | −0.337 |
| HA | CACGCA | 1330.18 | 617 | 0.464 | −0.768 |
| HA | CACGCT | 1519.52 | 549 | 0.361 | −1.018 |
| HC | CACTGC | 1778.65 | 2629 | 1.478 | 0.391 |
| HC | CACTGT | 1498.13 | 1717 | 1.146 | 0.136 |
| HC | CATTGT | 1086.40 | 673 | 0.619 | −0.479 |
| HC | CATTGC | 1289.82 | 634 | 0.492 | −0.710 |
| HD | CATGAT | 1329.76 | 2349 | 1.766 | 0.569 |
| HD | CATGAC | 1502.11 | 2329 | 1.550 | 0.439 |
| HD | CACGAC | 2071.40 | 1343 | 0.648 | −0.433 |
| HD | CACGAT | 1833.73 | 716 | 0.390 | −0.940 |
| HE | CATGAA | 1769.46 | 3512 | 1.985 | 0.686 |
| HE | CATGAG | 2366.33 | 3307 | 1.398 | 0.335 |
| HE | CACGAG | 3263.15 | 2230 | 0.683 | −0.381 |
| HE | CACGAA | 2440.07 | 790 | 0.324 | −1.128 |
| HF | CACTTC | 2538.66 | 3116 | 1.227 | 0.205 |
| HF | CATTTT | 1608.41 | 1806 | 1.123 | 0.116 |
| HF | CACTTT | 2217.98 | 1884 | 0.849 | −0.163 |
| HF | CATTTC | 1840.95 | 1400 | 0.760 | −0.274 |
| HG | CATGGA | 1246.72 | 2238 | 1.795 | 0.585 |
| HG | CATGGT | 807.15 | 1426 | 1.767 | 0.569 |
| HG | CATGGG | 1217.11 | 1849 | 1.519 | 0.418 |
| HG | CATGGC | 1690.37 | 2320 | 1.372 | 0.317 |
| HG | CACGGC | 2331.01 | 1680 | 0.721 | −0.328 |
| HG | CACGGG | 1678.38 | 1184 | 0.705 | −0.349 |
| HG | CACGGT | 1113.05 | 468 | 0.420 | −0.866 |
| HG | CACGGA | 1719.21 | 638 | 0.371 | −0.991 |
| HH | CACCAC | 2269.33 | 2795 | 1.232 | 0.208 |
| HH | CATCAT | 1193.37 | 1250 | 1.047 | 0.046 |
| HH | CACCAT | 1645.65 | 1453 | 0.883 | −0.125 |
| HH | CATCAC | 1645.65 | 1256 | 0.763 | −0.270 |
| HI | CACATC | 2433.52 | 3538 | 1.454 | 0.374 |
| HI | CACATT | 1924.40 | 1924 | 1.000 | 0.000 |
| HI | CACATA | 885.05 | 867 | 0.980 | −0.021 |
| HI | CATATT | 1395.51 | 1260 | 0.903 | −0.102 |
| HI | CATATA | 641.81 | 552 | 0.860 | −0.151 |
| HI | CATATC | 1764.71 | 904 | 0.512 | −0.669 |
| HK | CACAAG | 3102.81 | 3928 | 1.266 | 0.236 |
| HK | CACAAA | 2395.79 | 2432 | 1.015 | 0.015 |
| HK | CATAAA | 1737.35 | 1690 | 0.973 | −0.028 |
| HK | CATAAG | 2250.06 | 1436 | 0.638 | −0.449 |
| HL | CATTTA | 707.71 | 1053 | 1.488 | 0.397 |
| HL | CATTTG | 1188.90 | 1485 | 1.249 | 0.222 |
| HL | CACCTG | 5042.69 | 6030 | 1.196 | 0.179 |
| HL | CACCTC | 2426.56 | 2850 | 1.175 | 0.161 |
| HL | CATCTT | 1213.36 | 1409 | 1.161 | 0.149 |
| HL | CACTTG | 1639.48 | 1700 | 1.037 | 0.036 |
| HL | CATCTA | 654.36 | 649 | 0.992 | −0.008 |
| HL | CACCTT | 1673.21 | 1499 | 0.896 | −0.110 |
| HL | CACCTA | 902.35 | 761 | 0.843 | −0.170 |
| HL | CATCTC | 1759.66 | 1422 | 0.808 | −0.213 |
| HL | CACTTA | 975.93 | 781 | 0.800 | −0.223 |
| HL | CATCTG | 3656.80 | 2202 | 0.602 | −0.507 |
| HM | CACATG | 2348.18 | 3023 | 1.287 | 0.253 |
| HM | CATATG | 1702.82 | 1028 | 0.604 | −0.505 |
| HN | CACAAC | 2031.88 | 2762 | 1.359 | 0.307 |
| HN | CACAAT | 1844.85 | 1832 | 0.993 | −0.007 |
| HN | CATAAT | 1337.83 | 1225 | 0.916 | −0.088 |
| HN | CATAAC | 1473.45 | 869 | 0.590 | −0.528 |
| HP | CACCCG | 846.94 | 1341 | 1.583 | 0.460 |
| HP | CATCCT | 1518.15 | 1770 | 1.166 | 0.153 |
| HP | CACCCC | 2337.46 | 2530 | 1.082 | 0.079 |
| HP | CATCCA | 1465.21 | 1577 | 1.076 | 0.074 |
| HP | CACCCA | 2020.51 | 1919 | 0.950 | −0.052 |
| HP | CACCCT | 2093.51 | 1859 | 0.888 | −0.119 |
| HP | CATCCC | 1695.05 | 1265 | 0.746 | −0.293 |
| HP | CATCCG | 614.18 | 330 | 0.537 | −0.621 |
| HQ | CATCAA | 1143.96 | 1358 | 1.187 | 0.172 |
| HQ | CACCAG | 4405.09 | 4761 | 1.081 | 0.078 |
| HQ | CATCAG | 3194.43 | 2957 | 0.926 | −0.077 |
| HQ | CACCAA | 1577.51 | 1245 | 0.789 | −0.237 |
| HR | CACAGG | 1447.19 | 1936 | 1.338 | 0.291 |
| HR | CACCGC | 1336.44 | 1772 | 1.326 | 0.282 |
| HR | CACAGA | 1474.12 | 1788 | 1.213 | 0.193 |
| HR | CACCGG | 1461.67 | 1772 | 1.212 | 0.193 |
| HR | CACCGT | 572.18 | 667 | 1.166 | 0.153 |
| HR | CATCGA | 574.58 | 627 | 1.091 | 0.087 |
| HR | CATCGT | 414.93 | 452 | 1.089 | 0.086 |
| HR | CACCGA | 792.34 | 855 | 1.079 | 0.076 |
| HR | CATCGG | 1059.96 | 729 | 0.688 | −0.374 |
| HR | CATAGA | 1068.98 | 635 | 0.594 | −0.521 |
| HR | CATCGC | 969.15 | 565 | 0.583 | −0.540 |
| HR | CATAGG | 1049.46 | 423 | 0.403 | −0.909 |
| HS | CACTCG | 551.81 | 880 | 1.595 | 0.467 |
| HS | CACAGC | 2364.16 | 3726 | 1.576 | 0.455 |
| HS | CACAGT | 1491.56 | 1957 | 1.312 | 0.272 |
| HS | CATTCA | 1064.20 | 1307 | 1.228 | 0.206 |
| HS | CATTCT | 1316.36 | 1517 | 1.152 | 0.142 |
| HS | CACTCC | 2086.72 | 1964 | 0.941 | −0.061 |
| HS | CACTCA | 1467.52 | 1318 | 0.898 | −0.107 |
| HS | CATTCC | 1513.23 | 1219 | 0.806 | −0.216 |
| HS | CACTCT | 1815.24 | 1231 | 0.678 | −0.388 |
| HS | CATAGT | 1081.63 | 710 | 0.656 | −0.421 |
| HS | CATTCG | 400.16 | 256 | 0.640 | −0.447 |
| HS | CATAGC | 1714.41 | 782 | 0.456 | −0.785 |
| HT | CACACG | 778.62 | 1526 | 1.960 | 0.673 |
| HT | CACACT | 1696.86 | 2036 | 1.200 | 0.182 |
| HT | CACACA | 1918.82 | 2255 | 1.175 | 0.161 |
| HT | CACACC | 2370.26 | 2537 | 1.070 | 0.068 |
| HT | CATACT | 1230.51 | 1306 | 1.061 | 0.060 |
| HT | CATACA | 1391.46 | 979 | 0.704 | −0.352 |
| HT | CATACC | 1718.84 | 806 | 0.469 | −0.757 |
| HT | CATACG | 564.63 | 225 | 0.398 | −0.920 |
| HV | CATGTT | 869.32 | 1563 | 1.798 | 0.587 |
| HV | CATGTA | 564.48 | 880 | 1.559 | 0.444 |
| HV | CATGTC | 1113.00 | 1607 | 1.444 | 0.367 |
| HV | CATGTG | 2201.86 | 2797 | 1.270 | 0.239 |
| HV | CACGTG | 3036.34 | 2579 | 0.849 | −0.163 |
| HV | CACGTC | 1534.82 | 1158 | 0.754 | −0.282 |
| HV | CACGTT | 1198.78 | 434 | 0.362 | −1.016 |
| HV | CACGTA | 778.41 | 279 | 0.358 | −1.026 |
| HW | CACTGG | 1602.74 | 2197 | 1.371 | 0.315 |
| HW | CATTGG | 1162.26 | 568 | 0.489 | −0.716 |
| HY | CACTCC | 1943.40 | 2385 | 1.227 | 0.205 |
| HY | CATTCT | 1145.15 | 1240 | 1.083 | 0.080 |
| HY | CACTAT | 1579.16 | 1378 | 0.873 | −0.136 |
| HY | CATTCC | 1409.29 | 1074 | 0.762 | −0.272 |
| IA | ATTGCT | 1886.56 | 3678 | 1.950 | 0.668 |
| IA | ATAGCA | 759.54 | 1446 | 1.904 | 0.644 |
| IA | ATTGCA | 1651.49 | 2818 | 1.706 | 0.534 |
| IA | ATAGCT | 867.65 | 1289 | 1.486 | 0.396 |
| IA | ATTGCC | 2868.63 | 3435 | 1.197 | 0.180 |
| IA | ATAGCC | 1319.32 | 1191 | 0.903 | −0.102 |
| IA | ATCGCG | 980.82 | 708 | 0.722 | −0.326 |
| IA | ATCGCC | 3627.56 | 2570 | 0.708 | −0.345 |
| IA | ATTGCG | 775.62 | 494 | 0.637 | −0.451 |
| IA | ATAGCG | 356.72 | 198 | 0.555 | −0.589 |
| IA | ATCGCA | 2088.41 | 831 | 0.398 | −0.922 |
| IA | ATCGCT | 2385.67 | 910 | 0.381 | −0.964 |
| IC | ATCTGC | 2115.05 | 3055 | 1.444 | 0.368 |
| IC | ATCTGT | 1781.48 | 2074 | 1.164 | 0.152 |
| IC | ATATGT | 647.91 | 731 | 1.128 | 0.121 |
| IC | ATTTGT | 1408.77 | 1197 | 0.850 | −0.163 |
| IC | ATATGC | 769.23 | 470 | 0.611 | −0.493 |
| IC | ATTTGC | 1672.56 | 868 | 0.519 | −0.656 |
| ID | ATTGAT | 2604.76 | 4341 | 1.667 | 0.511 |
| ID | ATAGAT | 1197.96 | 1947 | 1.625 | 0.486 |
| ID | ATTGAC | 2942.37 | 3938 | 1.338 | 0.291 |
| ID | ATAGAC | 1353.23 | 1476 | 1.091 | 0.087 |
| ID | ATCGAC | 3720.81 | 2270 | 0.610 | −0.494 |
| ID | ATCGAT | 3293.87 | 1141 | 0.346 | −1.060 |
| IE | ATAGAA | 1371.51 | 2939 | 2.143 | 0.762 |
| IE | ATTGAA | 2982.12 | 5518 | 1.850 | 0.615 |
| IE | ATTGAG | 3988.04 | 4634 | 1.162 | 0.150 |
| IE | ATAGAG | 1834.15 | 1898 | 1.035 | 0.034 |
| IE | ATCGAG | 5043.12 | 3007 | 0.596 | −0.517 |
| IE | ATCGAA | 3771.07 | 994 | 0.264 | −1.333 |
| IF | ATATTT | 1144.73 | 1929 | 1.685 | 0.522 |
| IF | ATCTTC | 3602.60 | 4836 | 1.342 | 0.294 |
| IF | ATTTTT | 2489.02 | 2226 | 0.894 | −0.112 |
| IF | ATCTTT | 3147.52 | 2779 | 0.883 | −0.125 |
| IF | ATATTC | 1310.24 | 886 | 0.676 | −0.391 |
| IF | ATTTTC | 2848.89 | 1887 | 0.662 | −0.412 |
| IG | ATTGGT | 1013.16 | 2102 | 2.075 | 0.730 |
| IG | ATTGGA | 1564.91 | 3151 | 2.014 | 0.700 |
| IG | ATAGGA | 719.72 | 1054 | 1.464 | 0.381 |
| IG | ATTGGG | 1527.75 | 2144 | 1.403 | 0.339 |
| IG | ATAGGT | 465.96 | 596 | 1.279 | 0.246 |
| IG | ATTGGC | 2121.81 | 2706 | 1.275 | 0.243 |
| IG | ATAGGG | 702.63 | 549 | 0.781 | −0.247 |
| IG | ATAGGC | 975.84 | 700 | 0.717 | −0.332 |
| IG | ATCGGG | 1931.93 | 1244 | 0.644 | −0.440 |
| IG | ATCGGC | 2683.15 | 1619 | 0.603 | −0.505 |
| IG | ATCGGT | 1281.20 | 498 | 0.389 | −0.945 |
| IG | ATCGGA | 1978.93 | 604 | 0.305 | −1.187 |
| IH | ATTCAT | 1622.93 | 2242 | 1.381 | 0.323 |
| IH | ATCCAC | 2830.09 | 3367 | 1.190 | 0.174 |
| IH | ATACAT | 746.40 | 760 | 1.018 | 0.018 |
| IH | ATCCAT | 2052.29 | 1814 | 0.884 | −0.123 |
| IH | ATTCAC | 2238.00 | 1778 | 0.794 | −0.230 |
| IH | ATACAC | 1029.28 | 558 | 0.542 | −0.612 |
| II | ATCATC | 3797.03 | 5979 | 1.575 | 0.454 |
| II | ATAATA | 502.24 | 700 | 1.394 | 0.332 |
| II | ATAATT | 1092.04 | 1309 | 1.199 | 0.181 |
| II | ATCATT | 3002.64 | 3321 | 1.106 | 0.101 |
| II | ATTATT | 2374.46 | 2157 | 0.908 | −0.096 |
| II | ATCATA | 1380.95 | 1183 | 0.857 | −0.155 |
| II | ATTATA | 1092.04 | 921 | 0.843 | −0.170 |
| II | ATAATC | 1380.95 | 715 | 0.518 | −0.658 |
| II | ATTATC | 3002.64 | 1340 | 0.446 | −0.807 |
| IK | ATAAAA | 1419.09 | 2244 | 1.581 | 0.458 |
| IK | ATCAAG | 5053.39 | 5884 | 1.164 | 0.152 |
| IK | ATAAAG | 1837.88 | 1943 | 1.057 | 0.056 |
| IK | ATTAAA | 3085.58 | 3107 | 1.007 | 0.007 |
| IK | ATCAAA | 3901.90 | 3830 | 0.982 | −0.019 |
| IK | ATTAAG | 3996.16 | 2286 | 0.572 | −0.559 |
| IL | ATTTTA | 977.08 | 1679 | 1.718 | 0.541 |
| IL | ATATTA | 449.37 | 723 | 1.609 | 0.476 |
| IL | ATTTTG | 1641.41 | 2339 | 1.425 | 0.354 |
| IL | ATTCTT | 1675.18 | 2271 | 1.356 | 0.304 |
| IL | ATCCTC | 3072.14 | 4017 | 1.308 | 0.268 |
| IL | ATCCTG | 6384.29 | 7754 | 1.215 | 0.194 |
| IL | ATTCTA | 903.41 | 1021 | 1.130 | 0.122 |
| IL | ATCTTG | 2075.66 | 2250 | 1.084 | 0.081 |
| IL | ATCCTA | 1142.42 | 1170 | 1.024 | 0.024 |
| IL | ATACTA | 415.49 | 416 | 1.001 | 0.001 |
| IL | ATCCTT | 2118.37 | 2058 | 0.972 | −0.029 |
| IL | ATATTG | 754.90 | 717 | 0.950 | −0.052 |
| IL | ATACTT | 770.44 | 726 | 0.942 | −0.059 |
| IL | ATCTTA | 1235.57 | 1077 | 0.872 | −0.137 |
| IL | ATTCTC | 2429.41 | 1918 | 0.789 | −0.236 |
| IL | ATTCTG | 5048.62 | 3005 | 0.595 | −0.519 |
| IL | ATACTC | 1117.32 | 458 | 0.410 | −0.892 |
| IL | ATACTG | 2321.92 | 934 | 0.402 | −0.911 |
| IM | ATCATG | 3206.80 | 4314 | 1.345 | 0.297 |
| IM | ATAATG | 1166.29 | 1196 | 1.025 | 0.025 |
| IM | ATTATG | 2535.90 | 1399 | 0.552 | −0.595 |
| IN | ATAAAT | 1088.42 | 1649 | 1.515 | 0.415 |
| IN | ATCAAC | 3296.07 | 4599 | 1.395 | 0.333 |
| IN | ATCAAT | 2992.68 | 2890 | 0.966 | −0.035 |
| IN | ATAAAC | 1198.76 | 1113 | 0.928 | −0.074 |
| IN | ATTAAT | 2366.58 | 1967 | 0.831 | −0.185 |
| IN | ATTAAC | 2606.49 | 1331 | 0.511 | −0.672 |
| IP | ATTCCT | 2051.78 | 2787 | 1.358 | 0.306 |
| IP | ATTCCA | 1980.23 | 2644 | 1.335 | 0.289 |
| IP | ATACCA | 910.73 | 1047 | 1.150 | 0.139 |
| IP | ATCCCC | 2896.94 | 3229 | 1.115 | 0.109 |
| IP | ATACCT | 943.64 | 995 | 1.054 | 0.053 |
| IP | ATCCCG | 1049.66 | 1073 | 1.022 | 0.022 |
| IP | ATCCCA | 2504.13 | 2366 | 0.945 | −0.057 |
| IP | ATCCCT | 2594.61 | 2451 | 0.945 | −0.057 |
| IP | ATTCCC | 2290.86 | 1775 | 0.775 | −0.255 |
| IP | ATACCC | 1053.60 | 610 | 0.579 | −0.547 |
| IP | ATTCCG | 830.06 | 386 | 0.465 | −0.766 |
| IP | ATACCG | 381.76 | 125 | 0.327 | −1.116 |
| IQ | ATACAA | 765.47 | 950 | 1.241 | 0.216 |
| IQ | ATTCAA | 1664.38 | 2045 | 1.229 | 0.206 |
| IQ | ATCCAG | 5877.26 | 6881 | 1.171 | 0.158 |
| IQ | ATTCAG | 4647.67 | 3987 | 0.858 | −0.153 |
| IQ | ATCCAA | 2104.71 | 1765 | 0.839 | −0.176 |
| IQ | ATACAG | 2137.52 | 1569 | 0.734 | −0.309 |
| IR | ATCCGC | 1552.18 | 2623 | 1.690 | 0.525 |
| IR | ATTCGA | 727.72 | 1142 | 1.569 | 0.451 |
| IR | ATCCGA | 920.25 | 1434 | 1.558 | 0.444 |
| IR | ATCCGT | 664.55 | 943 | 1.419 | 0.350 |
| IR | ATAAGA | 622.67 | 877 | 1.408 | 0.342 |
| IR | ATCCGG | 1697.63 | 2265 | 1.334 | 0.288 |
| IR | ATTCGT | 525.51 | 677 | 1.288 | 0.253 |
| IR | ATCAGA | 1712.09 | 1680 | 0.981 | −0.019 |
| IR | ATCAGG | 1680.81 | 1513 | 0.900 | −0.105 |
| IR | ATAAGG | 611.30 | 547 | 0.895 | −0.111 |
| IR | ATACGT | 241.69 | 213 | 0.881 | −0.126 |
| IR | ATACGA | 334.69 | 292 | 0.872 | −0.136 |
| IR | ATTCGG | 1342.46 | 907 | 0.676 | −0.392 |
| IR | ATTAGA | 1353.90 | 900 | 0.665 | −0.408 |
| IR | ATTCGC | 1227.45 | 780 | 0.635 | −0.453 |
| IR | ATACGG | 617.42 | 260 | 0.421 | −0.865 |
| IR | ATTAGG | 1329.16 | 503 | 0.378 | −0.972 |
| IR | ATACGC | 564.52 | 170 | 0.301 | −1.200 |
| IS | ATCTCC | 2689.59 | 3743 | 1.392 | 0.330 |
| IS | ATATCA | 687.92 | 954 | 1.387 | 0.327 |
| IS | ATCAGC | 3047.17 | 3998 | 1.312 | 0.272 |
| IS | ATTTCT | 1850.19 | 2423 | 1.310 | 0.270 |
| IS | ATTTCA | 1495.77 | 1957 | 1.308 | 0.269 |
| IS | ATCAGT | 1922.48 | 2287 | 1.190 | 0.174 |
| IS | ATATCT | 850.92 | 1012 | 1.189 | 0.173 |
| IS | ATCTCG | 711.23 | 773 | 1.087 | 0.083 |
| IS | ATAAGT | 699.19 | 695 | 0.994 | −0.006 |
| IS | ATCTCT | 2339.68 | 2317 | 0.990 | −0.010 |
| IS | ATCTCA | 1891.49 | 1767 | 0.934 | −0.068 |
| IS | ATTTCC | 2126.89 | 1795 | 0.844 | −0.170 |
| IS | ATATCC | 978.18 | 703 | 0.719 | −0.330 |
| IS | ATTAGT | 1520.28 | 906 | 0.596 | −0.518 |
| IS | ATAAGC | 1108.24 | 636 | 0.574 | −0.555 |
| IS | ATATCG | 258.67 | 132 | 0.510 | −0.673 |
| IS | ATTTCG | 562.43 | 255 | 0.453 | −0.791 |
| IS | ATTAGC | 2409.67 | 797 | 0.331 | −1.106 |
| IT | ATCACC | 3094.94 | 4722 | 1.526 | 0.422 |
| IT | ATCACG | 1016.68 | 1306 | 1.285 | 0.250 |
| IT | ATAACT | 805.82 | 1009 | 1.252 | 0.225 |
| IT | ATCACT | 2215.66 | 2751 | 1.242 | 0.216 |
| IT | ATCACA | 2505.48 | 2989 | 1.193 | 0.176 |
| IT | ATAACA | 911.22 | 1079 | 1.184 | 0.169 |
| IT | ATTACT | 1752.12 | 1369 | 0.781 | −0.247 |
| IT | ATTACA | 1981.30 | 1531 | 0.773 | −0.258 |
| IT | ATAACC | 1125.61 | 741 | 0.658 | −0.418 |
| IT | ATAACG | 369.76 | 204 | 0.552 | −0.595 |
| IT | ATTACC | 2447.44 | 1083 | 0.443 | −0.815 |
| IT | ATTACG | 803.98 | 246 | 0.306 | −1.184 |
| IV | ATTGTT | 1261.28 | 2414 | 1.914 | 0.649 |
| IV | ATTGTA | 819.00 | 1478 | 1.805 | 0.590 |
| IV | ATAGTA | 376.67 | 645 | 1.712 | 0.538 |
| IV | ATAGTT | 580.08 | 877 | 1.512 | 0.413 |
| IV | ATTGTC | 1614.84 | 2315 | 1.434 | 0.360 |
| IV | ATTGTG | 3194.65 | 3762 | 1.178 | 0.163 |
| IV | ATCGTC | 2042.07 | 1679 | 0.822 | −0.196 |
| IV | ATAGTG | 1469.26 | 1196 | 0.814 | −0.206 |
| IV | ATAGTC | 742.69 | 575 | 0.774 | −0.256 |
| IV | ATCGTG | 4039.83 | 2922 | 0.723 | −0.324 |
| IV | ATCGTA | 1035.67 | 361 | 0.349 | −1.054 |
| IV | ATCGTT | 1594.97 | 547 | 0.343 | −1.070 |
| IW | ATCTGG | 1887.23 | 2427 | 1.286 | 0.252 |
| IW | ATATGG | 686.37 | 622 | 0.906 | −0.098 |
| IW | ATTTGG | 1492.40 | 1017 | 0.681 | −0.384 |
| IY | ATCTAC | 2708.47 | 3486 | 1.287 | 0.252 |
| IY | ATATAT | 800.43 | 953 | 1.191 | 0.174 |
| IY | ATTTAT | 1740.39 | 1984 | 1.140 | 0.131 |
| IY | ATCTAT | 2200.83 | 2196 | 0.998 | −0.002 |
| IY | ATTTAC | 2141.83 | 1403 | 0.655 | −0.423 |
| IY | ATATAC | 985.05 | 555 | 0.563 | −0.574 |
| KA | AAAGCA | 3029.93 | 4322 | 1.426 | 0.355 |
| KA | AAAGCT | 3461.21 | 4262 | 1.231 | 0.208 |
| KA | AAGGCC | 6816.15 | 6676 | 0.979 | −0.021 |
| KA | AAGGCG | 1842.96 | 1790 | 0.971 | −0.029 |
| KA | AAGGCA | 3924.10 | 3654 | 0.931 | −0.071 |
| KA | AAAGCC | 5262.99 | 4742 | 0.901 | −0.104 |
| KA | AAGGCT | 4482.65 | 4032 | 0.899 | −0.106 |
| KA | AAAGCG | 1423.01 | 765 | 0.538 | −0.621 |
| KC | AAATGT | 1815.55 | 2671 | 1.471 | 0.386 |
| KC | AAGTGT | 2351.33 | 2267 | 0.964 | −0.037 |
| KC | AAGTGC | 2791.62 | 2498 | 0.895 | −0.111 |
| KC | AAATGC | 2155.50 | 1678 | 0.778 | −0.250 |
| KD | AAAGAT | 4684.00 | 6115 | 1.306 | 0.267 |
| KD | AAGGAC | 6852.58 | 6836 | 0.998 | −0.002 |
| KD | AAGGAT | 6066.30 | 5379 | 0.887 | −0.120 |
| KD | AAAGAC | 5291.12 | 4564 | 0.863 | −0.148 |
| KE | AAAGAA | 6989.41 | 9895 | 1.416 | 0.348 |
| KE | AAGGAG | 12105.47 | 12287 | 1.015 | 0.015 |
| KE | AAGGAA | 9052.06 | 8366 | 0.924 | −0.079 |
| KE | AAAGAG | 9347.06 | 6946 | 0.743 | −0.297 |
| KF | AAATTT | 2631.62 | 3140 | 1.193 | 0.177 |
| KF | AAGTTT | 3408.25 | 3638 | 1.067 | 0.065 |
| KF | AAGTTC | 3901.02 | 3950 | 1.013 | 0.012 |
| KF | AAATTC | 3012.11 | 2225 | 0.739 | −0.303 |
| KG | AAAGGA | 2672.15 | 4509 | 1.687 | 0.523 |
| KG | AAAGGT | 1730.00 | 2402 | 1.388 | 0.328 |
| KG | AAAGGC | 3623.06 | 3435 | 0.948 | −0.053 |
| KG | AAAGGG | 2608.69 | 2465 | 0.945 | −0.057 |
| KG | AAGGGC | 4692.27 | 4309 | 0.918 | −0.085 |
| KG | AAGGGT | 2240.55 | 1978 | 0.883 | −0.125 |
| KG | AAGGGG | 3378.54 | 2740 | 0.811 | −0.209 |
| KG | AAGGGA | 3460.73 | 2568 | 0.742 | −0.298 |
| KH | AAACAT | 1929.29 | 2356 | 1.221 | 0.200 |
| KH | AAGCAC | 3445.60 | 3583 | 1.040 | 0.039 |
| KH | AAGCAT | 2498.64 | 2430 | 0.973 | −0.028 |
| KH | AAACAC | 2660.47 | 2165 | 0.814 | −0.206 |
| KI | AAAATA | 1547.96 | 2667 | 1.723 | 0.544 |
| KI | AAAATT | 3365.76 | 3894 | 1.157 | 0.146 |
| KI | AAGATC | 5512.26 | 5523 | 1.002 | 0.002 |
| KI | AAGATA | 2004.77 | 1943 | 0.969 | −0.031 |
| KI | AAGATT | 4359.03 | 3732 | 0.856 | −0.155 |
| KI | AAAATC | 4256.21 | 3287 | 0.772 | −0.258 |
| KK | AAGAAG | 11070.03 | 13815 | 1.248 | 0.222 |
| KK | AAGAAA | 8547.55 | 10129 | 1.185 | 0.170 |
| KK | AAAAAG | 8547.55 | 6145 | 0.719 | −0.330 |
| KK | AAAAAA | 6599.86 | 4676 | 0.708 | −0.345 |
| KL | AAATTA | 1273.72 | 2084 | 1.636 | 0.492 |
| KL | AAACTA | 1177.70 | 1750 | 1.486 | 0.396 |
| KL | AAACTT | 2183.78 | 3014 | 1.380 | 0.322 |
| KL | AAGCTG | 8523.68 | 9600 | 1.126 | 0.119 |
| KL | AAGCTA | 1525.25 | 1660 | 1.088 | 0.085 |
| KL | AAGCTC | 4101.62 | 4076 | 0.994 | −0.006 |
| KL | AAATTG | 2139.75 | 2113 | 0.987 | −0.013 |
| KL | AAGCTT | 2828.24 | 2772 | 0.980 | −0.020 |
| KL | AAGTTA | 1649.61 | 1459 | 0.884 | −0.123 |
| KL | AAACTC | 3167.00 | 2653 | 0.838 | −0.177 |
| KL | AAGTTG | 2771.21 | 2280 | 0.823 | −0.195 |
| KL | AAACTG | 6581.43 | 4462 | 0.678 | −0.389 |
| KM | AAGATG | 5479.27 | 5650 | 1.031 | 0.031 |
| KM | AAAATG | 4230.73 | 4060 | 0.960 | −0.041 |
| KN | AAAAAT | 3683.47 | 4378 | 1.189 | 0.173 |
| KN | AAGAAC | 5254.13 | 5515 | 1.050 | 0.048 |
| KN | AAGAAT | 4770.51 | 4618 | 0.968 | −0.032 |
| KN | AAAAAC | 4056.89 | 3254 | 0.802 | −0.221 |
| KP | AAACCA | 2803.51 | 3370 | 1.202 | 0.184 |
| KP | AAGCCC | 4200.41 | 4673 | 1.113 | 0.107 |
| KP | AAGCCA | 3630.85 | 4035 | 1.111 | 0.106 |
| KP | AAACCT | 2904.80 | 3118 | 1.073 | 0.071 |
| KP | AAGCCG | 1521.96 | 1544 | 1.014 | 0.014 |
| KP | AAGCCT | 3762.04 | 3396 | 0.903 | −0.102 |
| KP | AAACCC | 3243.28 | 2624 | 0.809 | −0.212 |
| KP | AAACCG | 1175.16 | 482 | 0.410 | −0.891 |
| KQ | AAACAA | 2178.87 | 3274 | 1.503 | 0.407 |
| KQ | AAGCAA | 2821.88 | 3177 | 1.126 | 0.119 |
| KQ | AAGCCG | 7879.90 | 8081 | 1.026 | 0.025 |
| KQ | AAACAG | 6084.35 | 4433 | 0.729 | −0.317 |
| KR | AAAAGA | 2247.57 | 3147 | 1.400 | 0.337 |
| KR | AAGAGG | 2857.67 | 3975 | 1.391 | 0.330 |
| KR | AAGAGA | 2910.85 | 3511 | 1.206 | 0.187 |
| KR | AAAAGG | 2206.51 | 2325 | 1.054 | 0.052 |
| KR | AAACGT | 872.39 | 862 | 0.988 | −0.012 |
| KR | AAGCGG | 2886.27 | 2828 | 0.980 | −0.020 |
| KR | AAGCGC | 2638.99 | 2532 | 0.959 | −0.041 |
| KR | AAACGA | 1208.07 | 1087 | 0.900 | −0.106 |
| KR | AAGCGT | 1129.84 | 978 | 0.866 | −0.144 |
| KR | AAGCGA | 1564.59 | 1325 | 0.847 | −0.166 |
| KR | AAACGG | 2228.59 | 1178 | 0.529 | −0.638 |
| KR | AAACGC | 2037.65 | 1041 | 0.511 | −0.672 |
| KS | AAATCA | 1871.14 | 2533 | 1.354 | 0.303 |
| KS | AAAAGT | 1901.80 | 2389 | 1.256 | 0.228 |
| KS | AAATCT | 2314.50 | 2793 | 1.207 | 0.188 |
| KS | AAGTCA | 2423.33 | 2566 | 1.059 | 0.057 |
| KS | AAGAGC | 3903.97 | 4045 | 1.036 | 0.035 |
| KS | AAGAGT | 2463.04 | 2459 | 0.998 | −0.002 |
| KS | AAGTCG | 911.22 | 904 | 0.992 | −0.008 |
| KS | AAGTCC | 3445.84 | 3100 | 0.900 | −0.106 |
| KS | AAGTCT | 2997.54 | 2675 | 0.892 | −0.114 |
| KS | AAATCC | 2660.65 | 2304 | 0.866 | −0.144 |
| KS | AAAAGC | 3014.39 | 2381 | 0.790 | −0.236 |
| KS | AAATCG | 703.58 | 462 | 0.657 | −0.421 |
| KT | AAAACA | 2831.74 | 3611 | 1.275 | 0.243 |
| KT | AAGACG | 1488.17 | 1790 | 1.203 | 0.185 |
| KT | AAAACT | 2504.18 | 2969 | 1.186 | 0.170 |
| KT | AAGACC | 4530.26 | 4475 | 0.988 | −0.012 |
| KT | AAGACA | 3667.42 | 3574 | 0.975 | −0.026 |
| KT | AAGACT | 3243.20 | 2876 | 0.887 | −0.120 |
| KT | AAAACC | 3497.97 | 2854 | 0.816 | −0.203 |
| KT | AAAACG | 1149.07 | 763 | 0.664 | −0.409 |
| KV | AAAGTA | 1317.00 | 2214 | 1.681 | 0.519 |
| KV | AAAGTT | 2028.22 | 3042 | 1.500 | 0.405 |
| KV | AAAGTC | 2596.78 | 2642 | 1.017 | 0.017 |
| KV | AAGGTG | 6653.25 | 6512 | 0.979 | −0.021 |
| KV | AAGGTC | 3363.11 | 3016 | 0.897 | −0.109 |
| KV | AAGGTT | 2626.77 | 2294 | 0.873 | −0.135 |
| KV | AAAGTG | 5137.21 | 4417 | 0.860 | −0.151 |
| KV | AAGGTA | 1705.66 | 1291 | 0.757 | −0.279 |
| KW | AAGTGG | 2598.56 | 2701 | 1.039 | 0.039 |
| KW | AAATGG | 2006.44 | 1904 | 0.949 | −0.052 |
| KY | AAATAT | 2319.32 | 2982 | 1.286 | 0.251 |
| KY | AAGTAC | 3696.62 | 3603 | 0.975 | −0.026 |
| KY | AAATAC | 2854.29 | 2763 | 0.968 | −0.033 |
| KY | AAGTAT | 3003.78 | 2526 | 0.841 | −0.173 |
| LA | CTGGCG | 2275.39 | 3643 | 1.601 | 0.471 |
| LA | TTGGCA | 1575.16 | 2350 | 1.492 | 0.400 |
| LA | CTGGCC | 8415.49 | 12456 | 1.480 | 0.392 |
| LA | TTGGCT | 1799.36 | 2643 | 1.469 | 0.384 |
| LA | TTAGCA | 937.64 | 1314 | 1.401 | 0.337 |
| LA | CTTGCT | 1836.39 | 2345 | 1.277 | 0.244 |
| LA | CTAGCA | 866.95 | 1107 | 1.277 | 0.244 |
| LA | CTTGCA | 1607.57 | 1861 | 1.158 | 0.146 |
| LA | TTAGCT | 1071.10 | 1239 | 1.157 | 0.146 |
| LA | CTGGCT | 5534.46 | 6333 | 1.144 | 0.135 |
| LA | CTAGCT | 990.35 | 1099 | 1.110 | 0.104 |
| LA | CTGGCA | 4844.85 | 5013 | 1.035 | 0.034 |
| LA | TTGGCC | 2736.04 | 2824 | 1.032 | 0.032 |
| LA | TTGGCG | 739.77 | 623 | 0.842 | −0.172 |
| LA | CTTGCC | 2792.34 | 2201 | 0.788 | −0.238 |
| LA | CTAGCC | 1505.89 | 1159 | 0.770 | −0.262 |
| LA | CTAGCG | 407.16 | 253 | 0.621 | −0.476 |
| LA | TTAGCC | 1628.68 | 941 | 0.578 | −0.549 |
| LA | CTTGCG | 755.00 | 346 | 0.458 | −0.780 |
| LA | TTAGCG | 440.36 | 198 | 0.450 | −0.799 |
| LA | CTCGCC | 4049.56 | 1527 | 0.377 | −0.975 |
| LA | CTCGCG | 1094.93 | 390 | 0.356 | −1.032 |
| LA | CTCGCT | 2663.20 | 605 | 0.227 | −1.482 |
| LA | CTCGCA | 2331.36 | 429 | 0.184 | −1.693 |
| LC | CTCTGC | 1769.27 | 3523 | 1.991 | 0.689 |
| LC | CTCTGT | 1490.23 | 2145 | 1.439 | 0.364 |
| LC | CTTTGT | 1027.58 | 1155 | 1.124 | 0.117 |
| LC | TTATGT | 599.35 | 627 | 1.046 | 0.045 |
| LC | CTGTGC | 3676.77 | 3517 | 0.957 | −0.044 |
| LC | TTGTGT | 1006.86 | 856 | 0.850 | −0.162 |
| LC | CTTTGC | 1219.99 | 974 | 0.798 | −0.225 |
| LC | CTGTGT | 3096.89 | 2370 | 0.765 | −0.268 |
| LC | CTATGT | 554.17 | 417 | 0.752 | −0.284 |
| LC | TTGTGC | 1195.39 | 722 | 0.604 | −0.504 |
| LC | TTATGC | 711.58 | 368 | 0.517 | −0.659 |
| LC | CTATGC | 657.93 | 332 | 0.505 | −0.684 |
| LD | TTGGAT | 2174.51 | 3688 | 1.696 | 0.528 |
| LD | TTAGAT | 1294.41 | 1977 | 1.527 | 0.424 |
| LD | CTGGAC | 7555.23 | 10531 | 1.394 | 0.332 |
| LD | CTAGAT | 1196.83 | 1584 | 1.323 | 0.280 |
| LD | TTGGAC | 2456.35 | 2775 | 1.130 | 0.122 |
| LD | CTTGAT | 2219.25 | 2463 | 1.110 | 0.104 |
| LD | CTGGAT | 6688.33 | 6912 | 1.033 | 0.033 |
| LD | CTAGAC | 1351.95 | 1390 | 1.028 | 0.028 |
| LD | CTTGAC | 2506.90 | 1832 | 0.731 | −0.314 |
| LD | TTAGAC | 1462.19 | 969 | 0.663 | −0.411 |
| LD | CTCGAC | 3635.60 | 981 | 0.270 | −1.310 |
| LD | CTCGAT | 3218.44 | 658 | 0.204 | −1.587 |
| LE | TTAGAA | 1739.66 | 3085 | 1.773 | 0.573 |
| LE | CTAGAA | 1608.51 | 2701 | 1.679 | 0.518 |
| LE | TTGGAA | 2922.49 | 4652 | 1.592 | 0.465 |
| LE | CTGGAG | 12021.09 | 18044 | 1.501 | 0.406 |
| LE | TTGGAG | 3908.29 | 4774 | 1.222 | 0.200 |
| LE | CTAGAG | 2151.09 | 2515 | 1.169 | 0.156 |
| LE | CTTGAA | 2982.63 | 3161 | 1.060 | 0.058 |
| LE | CTGGAA | 8988.96 | 7642 | 0.850 | −0.162 |
| LE | TTAGAG | 2326.48 | 1873 | 0.805 | −0.217 |
| LE | CTTGAG | 3988.72 | 2484 | 0.623 | −0.474 |
| LE | CTCGAG | 5784.58 | 1305 | 0.226 | −1.489 |
| LE | CTCGAA | 4325.51 | 512 | 0.118 | −2.134 |
| LF | CTCTTC | 2629.18 | 6495 | 2.470 | 0.904 |
| LF | TTATTT | 923.85 | 1405 | 1.521 | 0.419 |
| LF | CTCTTT | 2297.07 | 3446 | 1.500 | 0.406 |
| LF | CTTTTT | 1583.93 | 1937 | 1.223 | 0.201 |
| LF | CTTTTC | 1812.93 | 1936 | 1.068 | 0.066 |
| LF | CTATTT | 854.20 | 876 | 1.026 | 0.025 |
| LF | TTGTTT | 1551.99 | 1544 | 0.995 | −0.005 |
| LF | CTGTTT | 4773.59 | 2957 | 0.619 | −0.479 |
| LF | CTGTTC | 5463.77 | 3119 | 0.571 | −0.561 |
| LF | TTATTC | 1057.42 | 583 | 0.551 | −0.595 |
| LF | TTGTTC | 1776.38 | 940 | 0.529 | −0.636 |
| LF | CTATTC | 977.70 | 464 | 0.475 | −0.745 |
| LG | CTTGGA | 1534.14 | 2667 | 1.738 | 0.553 |
| LG | CTTGGT | 993.23 | 1579 | 1.590 | 0.464 |
| LG | CTGGGC | 6268.87 | 9794 | 1.562 | 0.446 |
| LG | CTAGGA | 827.35 | 1087 | 1.314 | 0.273 |
| LG | CTTGGG | 1497.70 | 1881 | 1.256 | 0.228 |
| LG | TTAGGA | 894.81 | 1114 | 1.245 | 0.219 |
| LG | CTGGGG | 4513.74 | 5602 | 1.241 | 0.216 |
| LG | TTGGGT | 973.20 | 1194 | 1.227 | 0.204 |
| LG | TTGGGA | 1503.20 | 1820 | 1.211 | 0.191 |
| LG | CTAGGT | 535.64 | 611 | 1.141 | 0.132 |
| LG | TTAGGT | 579.32 | 611 | 1.055 | 0.053 |
| LG | TTGGGG | 1467.50 | 1452 | 0.989 | −0.011 |
| LG | CTGGGT | 2993.37 | 2947 | 0.985 | −0.016 |
| LG | CTTGGC | 2080.08 | 2009 | 0.966 | −0.035 |
| LG | CTAGGG | 807.70 | 766 | 0.948 | −0.053 |
| LG | TTGGGC | 2038.13 | 1786 | 0.876 | −0.132 |
| LG | CTGGGA | 4623.54 | 4034 | 0.872 | −0.136 |
| LG | CTAGGC | 1121.77 | 940 | 0.838 | −0.177 |
| LG | TTAGGG | 873.56 | 529 | 0.606 | −0.502 |
| LG | CTCGGG | 2172.02 | 1076 | 0.495 | −0.702 |
| LG | CTCGGC | 3016.60 | 1313 | 0.435 | −0.832 |
| LG | TTAGGC | 1213.24 | 507 | 0.418 | −0.873 |
| LG | CTCGGT | 1440.42 | 365 | 0.253 | −1.373 |
| LG | CTCGGA | 2224.86 | 510 | 0.229 | −1.473 |
| LH | CTTCAT | 1127.31 | 1980 | 1.756 | 0.563 |
| LH | TTACAT | 657.52 | 935 | 1.422 | 0.352 |
| LH | CTACAT | 607.95 | 741 | 1.219 | 0.198 |
| LH | CTGCAC | 4685.05 | 5459 | 1.165 | 0.153 |
| LH | CTCCAC | 2254.46 | 2204 | 0.978 | −0.023 |
| LH | CTTCAC | 1554.55 | 1490 | 0.958 | −0.042 |
| LH | CTCCAT | 1634.86 | 1521 | 0.930 | −0.072 |
| LH | CTACAC | 838.36 | 777 | 0.927 | −0.076 |
| LH | TTGCAT | 1104.58 | 1017 | 0.921 | −0.083 |
| LH | TTGCAC | 1523.20 | 1140 | 0.748 | −0.290 |
| LH | CTGCAT | 3397.45 | 2394 | 0.705 | −0.350 |
| LH | TTACAC | 906.71 | 634 | 0.699 | −0.358 |
| LI | CTCATC | 2602.42 | 6250 | 2.402 | 0.876 |
| LI | TTAATA | 380.66 | 798 | 2.096 | 0.740 |
| LI | TTAATT | 827.68 | 1290 | 1.559 | 0.444 |
| LI | CTCATT | 2057.96 | 3117 | 1.515 | 0.415 |
| LI | CTAATA | 351.96 | 516 | 1.466 | 0.383 |
| LI | CTAATT | 765.28 | 952 | 1.244 | 0.218 |
| LI | CTTATT | 1419.05 | 1761 | 1.241 | 0.216 |
| LI | TTGATA | 639.48 | 791 | 1.237 | 0.213 |
| LI | TTGATT | 1390.44 | 1468 | 1.056 | 0.054 |
| LI | CTTATA | 652.64 | 683 | 1.047 | 0.045 |
| LI | CTCATA | 946.48 | 919 | 0.971 | −0.029 |
| LI | CTTATC | 1794.48 | 1189 | 0.663 | −0.412 |
| LI | TTGATC | 1758.29 | 1135 | 0.646 | −0.438 |
| LI | CTGATC | 5408.15 | 3356 | 0.621 | −0.477 |
| LI | CTGATT | 4276.70 | 2639 | 0.617 | −0.483 |
| LI | CTGATA | 1966.91 | 1193 | 0.607 | −0.500 |
| LI | TTAATC | 1046.66 | 633 | 0.605 | −0.503 |
| LI | CTAATC | 967.75 | 563 | 0.582 | −0.542 |
| LK | TTAAAA | 1429.91 | 2557 | 1.788 | 0.581 |
| LK | CTAAAA | 1322.10 | 1842 | 1.393 | 0.332 |
| LK | TTGAAA | 2402.12 | 3193 | 1.329 | 0.285 |
| LK | CTCAAG | 4604.55 | 6048 | 1.313 | 0.273 |
| LK | CTAAAG | 1712.27 | 2078 | 1.214 | 0.194 |
| LK | TTAAAG | 1851.89 | 2128 | 1.149 | 0.139 |
| LK | CTGAAG | 9568.82 | 10212 | 1.067 | 0.065 |
| LK | TTGAAG | 3111.01 | 3222 | 1.036 | 0.035 |
| LK | CTCAAA | 3555.33 | 2768 | 0.779 | −0.250 |
| LK | CTTAAA | 2451.55 | 1850 | 0.755 | −0.282 |
| LK | CTGAAA | 7388.42 | 5227 | 0.707 | −0.346 |
| LK | CTTAAG | 3175.03 | 1448 | 0.456 | −0.785 |
| LL | TTATTA | 500.55 | 802 | 1.602 | 0.471 |
| LL | CTTCTA | 793.49 | 1132 | 1.427 | 0.355 |
| LL | CTTCTT | 1471.36 | 2099 | 1.427 | 0.355 |
| LL | CTTTTA | 858.19 | 1203 | 1.402 | 0.338 |
| LL | CTGCTG | 13364.10 | 18236 | 1.365 | 0.311 |
| LL | CTTTTG | 1441.69 | 1945 | 1.349 | 0.299 |
| LL | TTACTA | 462.82 | 608 | 1.314 | 0.273 |
| LL | CTCCTC | 3094.54 | 3800 | 1.228 | 0.205 |
| LL | CTCCTG | 6430.85 | 7786 | 1.211 | 0.191 |
| LL | TTACTT | 858.19 | 1039 | 1.211 | 0.191 |
| LL | TTGCTA | 777.49 | 929 | 1.195 | 0.178 |
| LL | CTGCTC | 6430.85 | 7550 | 1.174 | 0.160 |
| LL | CTACTA | 427.93 | 474 | 1.108 | 0.102 |
| LL | CTTCTC | 2133.82 | 2292 | 1.074 | 0.072 |
| LL | CTACTT | 793.49 | 839 | 1.057 | 0.056 |
| LL | CTCTTG | 2090.79 | 2131 | 1.019 | 0.019 |
| LL | TTGCTT | 1441.69 | 1464 | 1.015 | 0.015 |
| LL | TTATTG | 840.89 | 818 | 0.973 | −0.028 |
| LL | CTCCTT | 2133.82 | 2034 | 0.953 | −0.048 |
| LL | TTGTTA | 840.89 | 771 | 0.917 | −0.087 |
| LL | TTGTTG | 1412.62 | 1289 | 0.912 | −0.092 |
| LL | CTCCTA | 1150.75 | 1034 | 0.899 | −0.107 |
| LL | TTGCTG | 4344.93 | 3820 | 0.879 | −0.129 |
| LL | CTTCTG | 4434.34 | 3837 | 0.865 | −0.145 |
| LL | CTGCTA | 2391.41 | 1913 | 0.800 | −0.223 |
| LL | CTCTTA | 1244.58 | 959 | 0.771 | −0.261 |
| LL | CTATTA | 462.82 | 354 | 0.765 | −0.268 |
| LL | CTGCTT | 4434.34 | 3148 | 0.710 | −0.343 |
| LL | TTGCTC | 2090.79 | 1440 | 0.689 | −0.373 |
| LL | CTACTC | 1150.75 | 792 | 0.688 | −0.374 |
| LL | CTATTG | 777.49 | 532 | 0.684 | −0.379 |
| LL | CTACTG | 2391.41 | 1583 | 0.662 | −0.413 |
| LL | CTGTTG | 4344.93 | 2615 | 0.602 | −0.508 |
| LL | TTACTC | 1244.58 | 657 | 0.528 | −0.639 |
| LL | TTACTG | 2586.40 | 1358 | 0.525 | −0.644 |
| LL | CTGTTA | 2586.40 | 953 | 0.368 | −0.998 |
| LM | CTCATG | 2631.41 | 4030 | 1.531 | 0.426 |
| LM | TTAATG | 1058.32 | 1228 | 1.160 | 0.149 |
| LM | CTAATG | 978.53 | 1101 | 1.125 | 0.118 |
| LM | TTGATG | 1777.88 | 1763 | 0.992 | −0.008 |
| LM | CTGATG | 5468.39 | 4470 | 0.817 | −0.202 |
| LM | CTTATG | 1814.47 | 1137 | 0.627 | −0.467 |
| LN | TTAAAT | 962.36 | 1926 | 2.001 | 0.694 |
| LN | CTCAAC | 2635.40 | 4681 | 1.776 | 0.574 |
| LN | CTAAAT | 889.81 | 1446 | 1.625 | 0.486 |
| LN | TTGAAT | 1616.68 | 2048 | 1.267 | 0.236 |
| LN | CTCAAT | 2392.82 | 2652 | 1.108 | 0.103 |
| LN | CTAAAC | 980.01 | 922 | 0.941 | −0.061 |
| LN | TTAAAC | 1059.92 | 965 | 0.910 | −0.094 |
| LN | CTTAAT | 1649.95 | 1441 | 0.873 | −0.135 |
| LN | TTGAAC | 1780.58 | 1541 | 0.865 | −0.145 |
| LN | CTGAAC | 5476.68 | 4308 | 0.787 | −0.240 |
| LN | CTGAAT | 4972.58 | 3413 | 0.686 | −0.376 |
| LN | CTTAAC | 1817.22 | 891 | 0.490 | −0.713 |
| LP | CTTCCT | 1728.14 | 2795 | 1.617 | 0.481 |
| LP | CTTCCA | 1667.88 | 2369 | 1.420 | 0.351 |
| LP | CTGCCC | 5815.10 | 7856 | 1.351 | 0.301 |
| LP | TTACCT | 1007.96 | 1244 | 1.234 | 0.210 |
| LP | CTGCCG | 2107.02 | 2489 | 1.181 | 0.167 |
| LP | TTACCA | 972.81 | 1140 | 1.172 | 0.159 |
| LP | CTCCCG | 1013.90 | 1184 | 1.168 | 0.155 |
| LP | TTGCCA | 1634.25 | 1897 | 1.161 | 0.149 |
| LP | CTACCT | 931.97 | 1045 | 1.121 | 0.114 |
| LP | TTGCCT | 1693.30 | 1800 | 1.063 | 0.061 |
| LP | CTTCCC | 1929.51 | 1889 | 0.979 | −0.021 |
| LP | CTACCA | 899.47 | 850 | 0.945 | −0.057 |
| LP | CTCCCA | 2418.82 | 2126 | 0.879 | −0.129 |
| LP | CTGCCT | 5208.23 | 4563 | 0.876 | −0.132 |
| LP | CTCCCT | 2506.21 | 2192 | 0.875 | −0.134 |
| LP | CTACCC | 1040.57 | 888 | 0.853 | −0.159 |
| LP | CTCCCC | 2798.25 | 2369 | 0.847 | −0.167 |
| LP | TTGCCC | 1890.60 | 1560 | 0.825 | −0.192 |
| LP | TTGCCG | 685.03 | 478 | 0.698 | −0.360 |
| LP | CTGCCA | 5026.60 | 3348 | 0.666 | −0.406 |
| LP | CTTCCG | 699.13 | 451 | 0.645 | −0.438 |
| LP | TTACCC | 1125.42 | 666 | 0.592 | −0.525 |
| LP | CTACCG | 377.04 | 211 | 0.560 | −0.580 |
| LP | TTACCG | 407.78 | 175 | 0.429 | −0.846 |
| LQ | TTACAA | 864.28 | 1290 | 1.493 | 0.401 |
| LQ | CTACAA | 799.12 | 1188 | 1.487 | 0.397 |
| LQ | CTTCAA | 1481.79 | 2098 | 1.416 | 0.348 |
| LQ | CTACAG | 2231.48 | 2674 | 1.198 | 0.181 |
| LQ | CTGCAG | 12470.36 | 14508 | 1.163 | 0.151 |
| LQ | CTTCAG | 4137.79 | 4363 | 1.054 | 0.053 |
| LQ | TTGCAA | 1451.91 | 1467 | 1.010 | 0.010 |
| LQ | CTCCAG | 6000.78 | 5430 | 0.905 | −0.100 |
| LQ | TTACAG | 2413.43 | 2107 | 0.873 | −0.136 |
| LQ | TTGCAG | 4054.36 | 3177 | 0.784 | −0.244 |
| LQ | CTCCAA | 2148.94 | 1524 | 0.709 | −0.344 |
| LQ | CTGCAA | 4465.77 | 2694 | 0.603 | −0.505 |
| LR | CTTCGA | 661.43 | 1365 | 2.064 | 0.725 |
| LR | CTTCGT | 477.64 | 784 | 1.641 | 0.496 |
| LR | CTGCGG | 3677.31 | 5467 | 1.487 | 0.397 |
| LR | TTAAGA | 717.74 | 1026 | 1.429 | 0.357 |
| LR | CTGCGC | 3362.26 | 4574 | 1.360 | 0.308 |
| LR | CTCCGA | 959.23 | 1289 | 1.344 | 0.295 |
| LR | CTCCGG | 1769.53 | 2229 | 1.260 | 0.231 |
| LR | CTAAGA | 663.63 | 821 | 1.237 | 0.213 |
| LR | CTCAGG | 1752.00 | 2047 | 1.168 | 0.156 |
| LR | CTTCGG | 1220.17 | 1415 | 1.160 | 0.148 |
| LR | CTCCGT | 692.69 | 771 | 1.113 | 0.107 |
| LR | TTACGA | 385.79 | 427 | 1.107 | 0.101 |
| LR | CTAAGG | 651.51 | 721 | 1.107 | 0.101 |
| LR | CTCCGC | 1617.93 | 1790 | 1.106 | 0.101 |
| LR | TTGAGA | 1205.75 | 1290 | 1.070 | 0.068 |
| LR | CTACGT | 257.59 | 275 | 1.068 | 0.065 |
| LR | CTACGA | 356.70 | 378 | 1.060 | 0.058 |
| LR | CTGAGG | 3640.88 | 3637 | 0.999 | −0.001 |
| LR | TTAAGG | 704.63 | 678 | 0.962 | −0.039 |
| LR | TTACGT | 278.59 | 264 | 0.948 | −0.054 |
| LR | CTGCGT | 1439.50 | 1363 | 0.947 | −0.055 |
| LR | TTGAGG | 1183.72 | 1080 | 0.912 | −0.092 |
| LR | CTACGG | 658.03 | 577 | 0.877 | −0.131 |
| LR | CTCAGA | 1784.60 | 1469 | 0.823 | −0.195 |
| LR | CTTCGC | 1115.63 | 819 | 0.734 | −0.309 |
| LR | CTACGC | 601.65 | 438 | 0.728 | −0.317 |
| LR | CTGCGA | 1993.40 | 1399 | 0.702 | −0.354 |
| LR | TTGCGT | 468.01 | 321 | 0.686 | −0.377 |
| LR | CTGAGA | 3708.63 | 2486 | 0.670 | −0.400 |
| LR | TTGCGG | 1195.56 | 772 | 0.646 | −0.437 |
| LR | TTGCGA | 648.09 | 418 | 0.645 | −0.439 |
| LR | CTTAGA | 1230.56 | 694 | 0.564 | −0.573 |
| LR | TTACGG | 711.68 | 383 | 0.538 | −0.620 |
| LR | TTGCGC | 1093.14 | 542 | 0.496 | −0.702 |
| LR | CTTAGG | 1208.08 | 503 | 0.416 | −0.876 |
| LR | TTACGC | 650.71 | 232 | 0.357 | −1.031 |
| LS | CTCAGC | 2740.30 | 5167 | 1.886 | 0.634 |
| LS | CTTTCT | 1450.83 | 2502 | 1.725 | 0.545 |
| LS | CTCTCC | 2418.72 | 4070 | 1.683 | 0.520 |
| LS | CTCTCG | 639.61 | 1016 | 1.588 | 0.463 |
| LS | CTCAGT | 1728.87 | 2589 | 1.498 | 0.404 |
| LS | TTATCA | 684.12 | 963 | 1.408 | 0.342 |
| LS | TTATCT | 846.22 | 1175 | 1.389 | 0.328 |
| LS | CTTTCA | 1172.91 | 1626 | 1.386 | 0.327 |
| LS | TTAAGT | 695.33 | 886 | 1.274 | 0.242 |
| LS | CTCTCT | 2104.05 | 2553 | 1.213 | 0.193 |
| LS | CTAAGT | 642.91 | 770 | 1.198 | 0.180 |
| LS | CTCTCA | 1701.00 | 2003 | 1.178 | 0.163 |
| LS | CTTTCC | 1667.81 | 1819 | 1.091 | 0.087 |
| LS | TTGTCA | 1149.26 | 1210 | 1.053 | 0.052 |
| LS | CTGTCG | 1329.18 | 1392 | 1.047 | 0.046 |
| LS | TTGTCT | 1421.58 | 1461 | 1.028 | 0.027 |
| LS | CTGAGC | 5694.68 | 5805 | 1.019 | 0.019 |
| LS | CTGTCC | 5026.41 | 4628 | 0.921 | −0.083 |
| LS | TTGAGT | 1168.09 | 1035 | 0.886 | −0.121 |
| LS | TTGTCC | 1634.18 | 1334 | 0.816 | −0.203 |
| LS | CTATCA | 632.54 | 512 | 0.809 | −0.211 |
| LS | CTAAGC | 1019.02 | 791 | 0.776 | −0.253 |
| LS | TTATCC | 972.78 | 727 | 0.747 | −0.291 |
| LS | CTGAGT | 3592.81 | 2665 | 0.742 | −0.299 |
| LS | CTTAGT | 1192.13 | 856 | 0.718 | −0.331 |
| LS | CTATCT | 782.42 | 557 | 0.712 | −0.340 |
| LS | CTGTCT | 4372.48 | 2950 | 0.675 | −0.394 |
| LS | CTTTCG | 441.04 | 291 | 0.660 | −0.416 |
| LS | TTGTCG | 432.14 | 278 | 0.643 | −0.441 |
| LS | CTGTCA | 3534.89 | 2228 | 0.630 | −0.462 |
| LS | TTGAGC | 1851.45 | 1128 | 0.609 | −0.496 |
| LS | CTATCC | 899.44 | 541 | 0.601 | −0.508 |
| LS | TTATCG | 257.24 | 152 | 0.591 | −0.526 |
| LS | TTAAGC | 1102.11 | 551 | 0.500 | −0.693 |
| LS | CTATCG | 237.85 | 102 | 0.429 | −0.847 |
| LS | CTTAGC | 1889.55 | 793 | 0.420 | −0.868 |
| LT | CTCACC | 2534.19 | 4959 | 1.957 | 0.671 |
| LT | CTCACG | 832.47 | 1510 | 1.814 | 0.595 |
| LT | TTAACA | 825.09 | 1163 | 1.410 | 0.343 |
| LT | CTCACT | 1814.22 | 2521 | 1.390 | 0.329 |
| LT | TTAACT | 729.65 | 969 | 1.328 | 0.284 |
| LT | CTAACT | 674.64 | 817 | 1.211 | 0.191 |
| LT | CTAACA | 762.89 | 898 | 1.177 | 0.163 |
| LT | CTCACA | 2051.52 | 2374 | 1.157 | 0.146 |
| LT | CTGACG | 1729.98 | 1795 | 1.038 | 0.037 |
| LT | TTGACT | 1225.76 | 1259 | 1.027 | 0.027 |
| LT | TTGACA | 1386.09 | 1401 | 1.011 | 0.011 |
| LT | CTTACT | 1250.98 | 1259 | 1.006 | 0.006 |
| LT | CTGACC | 5266.36 | 5160 | 0.980 | −0.020 |
| LT | CTTACA | 1414.61 | 1109 | 0.784 | −0.243 |
| LT | CTGACT | 3770.17 | 2808 | 0.745 | −0.295 |
| LT | TTGACC | 1712.20 | 1235 | 0.721 | −0.327 |
| LT | CTAACC | 942.38 | 678 | 0.719 | −0.329 |
| LT | TTGACG | 562.45 | 399 | 0.709 | −0.343 |
| LT | CTGACA | 4263.32 | 3003 | 0.704 | −0.350 |
| LT | CTAACG | 309.57 | 215 | 0.695 | −0.365 |
| LT | TTAACC | 1019.22 | 687 | 0.674 | −0.394 |
| LT | CTTACC | 1747.43 | 1104 | 0.632 | −0.459 |
| LT | TTAACG | 334.81 | 164 | 0.490 | −0.714 |
| LT | CTTACG | 574.02 | 247 | 0.430 | −0.843 |
| LV | CTTGTT | 1029.60 | 1741 | 1.691 | 0.525 |
| LV | TTAGTA | 389.95 | 602 | 1.544 | 0.434 |
| LV | TTGGTA | 655.07 | 980 | 1.496 | 0.403 |
| LV | CTTGTA | 668.56 | 993 | 1.485 | 0.396 |
| LV | CTGGTG | 7859.41 | 11424 | 1.454 | 0.374 |
| LV | CTAGTA | 360.55 | 519 | 1.439 | 0.364 |
| LV | TTGGTT | 1008.84 | 1427 | 1.414 | 0.347 |
| LV | CTTGTC | 1318.22 | 1541 | 1.169 | 0.156 |
| LV | TTAGTT | 600.53 | 690 | 1.149 | 0.139 |
| LV | CTGGTC | 3972.81 | 4541 | 1.143 | 0.134 |
| LV | TTGGTG | 2555.25 | 2882 | 1.128 | 0.120 |
| LV | CTAGTT | 555.26 | 580 | 1.045 | 0.044 |
| LV | TTGGTC | 1291.64 | 1345 | 1.041 | 0.040 |
| LV | CTTGTG | 2607.83 | 2540 | 0.974 | −0.026 |
| LV | CTAGTG | 1406.38 | 1272 | 0.904 | −0.100 |
| LV | CTGGTA | 2014.87 | 1720 | 0.854 | −0.158 |
| LV | CTGGTT | 3102.98 | 2576 | 0.830 | −0.186 |
| LV | CTAGTC | 710.90 | 551 | 0.775 | −0.255 |
| LV | TTAGTG | 1521.06 | 947 | 0.623 | −0.474 |
| LV | TTAGTC | 768.87 | 416 | 0.541 | −0.614 |
| LV | CTCGTC | 1911.73 | 1013 | 0.530 | −0.635 |
| LV | CTCGTG | 3781.97 | 1691 | 0.447 | −0.805 |
| LV | CTCGTT | 1493.16 | 373 | 0.250 | −1.387 |
| LV | CTCGTA | 969.56 | 191 | 0.197 | −1.625 |
| LW | CTCTGG | 1742.64 | 2796 | 1.604 | 0.473 |
| LW | CTGTGG | 3621.43 | 3365 | 0.929 | −0.073 |
| LW | CTTTGG | 1201.63 | 1018 | 0.847 | −0.166 |
| LW | CTATGG | 648.03 | 501 | 0.773 | −0.257 |
| LW | TTATGG | 700.87 | 535 | 0.763 | −0.270 |
| LW | TTGTGG | 1177.40 | 877 | 0.745 | −0.295 |
| LY | CTCTAC | 2082.09 | 4204 | 2.019 | 0.703 |
| LY | TTATAT | 680.44 | 1022 | 1.502 | 0.407 |
| LY | CTCTAT | 1691.85 | 2487 | 1.470 | 0.385 |
| LY | CTTTAT | 1166.60 | 1591 | 1.364 | 0.310 |
| LY | CTATAT | 629.14 | 596 | 0.947 | −0.054 |
| LY | TTGTAT | 1143.08 | 1063 | 0.930 | −0.073 |
| LY | CTGTAC | 4326.84 | 3390 | 0.783 | −0.244 |
| LY | CTTTAC | 1435.69 | 1069 | 0.745 | −0.295 |
| LY | TTGTAC | 1406.74 | 1006 | 0.715 | −0.335 |
| LY | TTATAC | 837.39 | 579 | 0.691 | −0.369 |
| LY | CTGTAT | 3515.88 | 2202 | 0.626 | −0.468 |
| LY | CTATAC | 774.26 | 481 | 0.621 | −0.476 |
| MA | ATGGCG | 1645.46 | 2370 | 1.440 | 0.365 |
| MA | ATGGCA | 3503.58 | 3580 | 1.022 | 0.022 |
| MA | ATGGCT | 4002.27 | 4003 | 1.000 | 0.000 |
| MA | ATGGCC | 6085.70 | 5284 | 0.868 | −0.141 |
| MC | ATGTGT | 1386.67 | 1448 | 1.044 | 0.043 |
| MC | ATGTGC | 1646.33 | 1585 | 0.963 | −0.038 |
| MD | ATGGAT | 4467.48 | 4634 | 1.037 | 0.037 |
| MD | ATGGAC | 5046.52 | 4880 | 0.967 | −0.034 |
| ME | ATGGAG | 8054.28 | 8223 | 1.021 | 0.021 |
| ME | ATGGAA | 6022.72 | 5854 | 0.972 | −0.028 |
| MF | ATGTTT | 2565.53 | 2833 | 1.104 | 0.099 |
| MF | ATGTTC | 2936.47 | 2669 | 0.909 | −0.096 |
| MG | ATGGGC | 3467.73 | 3533 | 1.019 | 0.019 |
| MG | ATGGGT | 1655.83 | 1675 | 1.012 | 0.012 |
| MG | ATGGGA | 2557.59 | 2526 | 0.988 | −0.012 |
| MG | ATGGGG | 2496.85 | 2444 | 0.979 | −0.021 |
| MH | ATGCAT | 1465.33 | 1478 | 1.009 | 0.009 |
| MH | ATGCAC | 2020.67 | 2008 | 0.994 | −0.006 |
| MI | ATGATT | 2305.40 | 2382 | 1.033 | 0.033 |
| MI | ATGATA | 1060.28 | 1094 | 1.032 | 0.031 |
| MI | ATGATC | 2915.32 | 2805 | 0.962 | −0.039 |
| MK | ATGAAG | 6107.32 | 6423 | 1.052 | 0.050 |
| MK | ATGAAA | 4715.68 | 4400 | 0.933 | −0.069 |
| ML | ATGCTG | 5938.40 | 6536 | 1.101 | 0.096 |
| ML | ATGCTA | 1062.63 | 1122 | 1.056 | 0.054 |
| ML | ATGTTG | 1930.69 | 1922 | 0.995 | −0.005 |
| ML | ATGTTA | 1149.28 | 1134 | 0.987 | −0.013 |
| ML | ATGCTT | 1970.42 | 1887 | 0.958 | −0.043 |
| ML | ATGCTC | 2857.58 | 2308 | 0.808 | −0.214 |
| MM | ATGATG | 3925.00 | 3925 | 1.000 | 0.000 |
| MN | ATGAAT | 3249.30 | 3301 | 1.016 | 0.016 |
| MN | ATGAAC | 3578.70 | 3527 | 0.986 | −0.015 |
| MP | ATGCCC | 2676.16 | 2752 | 1.028 | 0.028 |
| MP | ATGCCA | 2313.29 | 2313 | 1.000 | 0.000 |
| MP | ATGCCT | 2396.87 | 2372 | 0.990 | −0.010 |
| MP | ATGCCG | 969.67 | 919 | 0.948 | −0.054 |
| MQ | ATGCAG | 5141.70 | 5165 | 1.005 | 0.005 |
| MQ | ATGCAA | 1841.30 | 1818 | 0.987 | −0.013 |
| MR | ATGAGG | 1626.37 | 2127 | 1.308 | 0.268 |
| MR | ATGAGA | 1656.63 | 1974 | 1.192 | 0.175 |
| MR | ATGCGG | 1642.64 | 1513 | 0.921 | −0.082 |
| MR | ATGCGT | 643.02 | 531 | 0.826 | −0.191 |
| MR | ATGCGA | 890.44 | 684 | 0.768 | −0.264 |
| MR | ATGCGC | 1501.91 | 1132 | 0.754 | −0.283 |
| MS | ATGTCG | 666.33 | 809 | 1.214 | 0.194 |
| MS | ATGTCT | 2191.95 | 2338 | 1.067 | 0.065 |
| MS | ATGTCA | 1772.07 | 1781 | 1.005 | 0.005 |
| MS | ATGTCC | 2519.77 | 2493 | 0.989 | −0.011 |
| MS | ATGAGT | 1801.10 | 1770 | 0.983 | −0.017 |
| MS | ATGAGC | 2854.78 | 2615 | 0.916 | −0.088 |
| MT | ATGACT | 2098.83 | 2195 | 1.046 | 0.045 |
| MT | ATGACC | 2931.75 | 2927 | 0.998 | −0.002 |
| MT | ATGACA | 2373.36 | 2337 | 0.985 | −0.015 |
| MT | ATGACG | 963.07 | 908 | 0.943 | −0.059 |
| MV | ATGGTG | 4813.46 | 5122 | 1.064 | 0.062 |
| MV | ATGGTT | 1900.41 | 1915 | 1.008 | 0.008 |
| MV | ATGGTA | 1234.00 | 1191 | 0.965 | −0.035 |
| MV | ATGGTC | 2433.13 | 2153 | 0.885 | −0.122 |
| MW | ATGTGG | 1876.00 | 1876 | 1.000 | 0.000 |
| MY | ATGTAC | 2354.66 | 2363 | 1.004 | 0.004 |
| MY | ATGTAT | 1913.34 | 1905 | 0.996 | −0.004 |
| NA | AATGCA | 1705.68 | 3344 | 1.961 | 0.673 |
| NA | AATGCT | 1948.47 | 3458 | 1.775 | 0.574 |
| NA | AATGCC | 2962.77 | 4259 | 1.438 | 0.363 |
| NA | AATGCG | 801.08 | 624 | 0.779 | −0.250 |
| NA | AACGCG | 882.29 | 661 | 0.749 | −0.289 |
| NA | AACGCC | 3263.12 | 1899 | 0.582 | −0.541 |
| NA | AACGCA | 1878.60 | 700 | 0.373 | −0.987 |
| NA | AACGCT | 2146.00 | 643 | 0.300 | −1.205 |
| NC | AACTGC | 1868.57 | 2826 | 1.512 | 0.414 |
| NC | AACTGT | 1573.86 | 2016 | 1.281 | 0.248 |
| NC | AATTGT | 1429.00 | 935 | 0.654 | −0.424 |
| NC | AATTGC | 1696.57 | 791 | 0.466 | −0.763 |
| ND | AATGAT | 2555.01 | 4420 | 1.730 | 0.548 |
| ND | AATGAC | 2886.18 | 4521 | 1.566 | 0.449 |
| ND | AACGAC | 3178.77 | 1654 | 0.520 | −0.653 |
| ND | AACGAT | 2814.03 | 839 | 0.298 | −1.210 |
| NE | AATGAA | 3381.19 | 7367 | 2.179 | 0.779 |
| NE | AATGAG | 4521.72 | 5796 | 1.282 | 0.248 |
| NE | AACGAG | 4980.12 | 2476 | 0.497 | −0.699 |
| NE | AACGAA | 3723.97 | 968 | 0.260 | −1.347 |
| NF | AACTTC | 3150.86 | 4259 | 1.352 | 0.301 |
| NF | AACTTT | 2752.85 | 2846 | 1.034 | 0.033 |
| NF | AATTTT | 2499.46 | 2350 | 0.940 | −0.062 |
| NF | AATTTC | 2860.84 | 1809 | 0.632 | −0.458 |
| NG | AATGGA | 2235.93 | 4484 | 2.005 | 0.696 |
| NG | AATGGT | 1447.59 | 2430 | 1.679 | 0.518 |
| NG | AATGGG | 2182.83 | 3202 | 1.467 | 0.383 |
| NG | AATGGC | 3031.62 | 4001 | 1.320 | 0.277 |
| NG | AACGGG | 2404.12 | 1508 | 0.627 | −0.466 |
| NG | AACGGC | 3338.95 | 1752 | 0.525 | −0.645 |
| NG | AACGGA | 2462.61 | 804 | 0.326 | −1.119 |
| NG | AACGGT | 1594.34 | 517 | 0.324 | −1.126 |
| NH | AACCAC | 2167.68 | 2776 | 1.281 | 0.247 |
| NH | AACCAT | 1571.93 | 1639 | 1.043 | 0.042 |
| NH | AATCAT | 1427.24 | 1456 | 1.020 | 0.020 |
| NH | AATCAC | 1968.15 | 1264 | 0.642 | −0.443 |
| NI | AACATC | 3876.27 | 5487 | 1.416 | 0.348 |
| NI | AACATT | 3065.31 | 3184 | 1.039 | 0.038 |
| NI | AATATA | 1280.01 | 1309 | 1.023 | 0.022 |
| NI | AACATA | 1409.77 | 1384 | 0.982 | −0.018 |
| NI | AATATT | 2783.16 | 2725 | 0.979 | −0.021 |
| NI | AATATC | 3519.48 | 1845 | 0.524 | −0.646 |
| NK | AACAAG | 4824.98 | 5918 | 1.227 | 0.204 |
| NK | AACAAA | 3725.54 | 4221 | 1.133 | 0.125 |
| NK | AATAAA | 3382.62 | 3607 | 1.066 | 0.064 |
| NK | AATAAG | 4380.86 | 2568 | 0.586 | −0.534 |
| NL | AATTTA | 1025.31 | 1571 | 1.532 | 0.427 |
| NL | AACCTC | 2807.78 | 3954 | 1.408 | 0.342 |
| NL | AACTTG | 1897.05 | 2429 | 1.280 | 0.247 |
| NL | AACCTG | 5834.92 | 6690 | 1.147 | 0.137 |
| NL | AATTTG | 1722.43 | 1947 | 1.130 | 0.123 |
| NL | AATCTT | 1757.88 | 1943 | 1.105 | 0.100 |
| NL | AACCTA | 1044.12 | 1135 | 1.087 | 0.083 |
| NL | AACCTT | 1936.08 | 2021 | 1.044 | 0.043 |
| NL | AACTTA | 1129.25 | 1129 | 1.000 | 0.000 |
| NL | AATCTA | 948.01 | 893 | 0.942 | −0.060 |
| NL | AATCTC | 2549.34 | 1713 | 0.672 | −0.398 |
| NL | AATCTG | 5297.84 | 2525 | 0.477 | −0.741 |
| NM | AACATG | 3351.76 | 4374 | 1.305 | 0.266 |
| NM | AATATG | 3043.24 | 2021 | 0.664 | −0.409 |
| NN | AACAAC | 3150.02 | 4430 | 1.406 | 0.341 |
| NN | AACAAT | 2860.08 | 2830 | 0.989 | −0.011 |
| NN | AATAAT | 2596.82 | 2424 | 0.933 | −0.069 |
| NN | AATAAC | 2860.08 | 1783 | 0.623 | −0.473 |
| NP | AACCCC | 2770.02 | 3474 | 1.254 | 0.226 |
| NP | AATCCA | 2174.02 | 2380 | 1.095 | 0.091 |
| NP | AACCCA | 2394.42 | 2612 | 1.091 | 0.087 |
| NP | AATCCT | 2252.58 | 2414 | 1.072 | 0.069 |
| NP | AACCCG | 1003.68 | 1048 | 1.044 | 0.043 |
| NP | AACCCT | 2480.94 | 2578 | 1.039 | 0.038 |
| NP | AATCCC | 2515.05 | 1641 | 0.652 | −0.427 |
| NP | AATCCG | 911.29 | 355 | 0.390 | −0.943 |
| NQ | AATCAA | 1516.57 | 1905 | 1.256 | 0.228 |
| NQ | AACCAA | 1670.31 | 1955 | 1.170 | 0.157 |
| NQ | AACCAG | 4664.22 | 5409 | 1.160 | 0.148 |
| NQ | AATCAG | 4234.90 | 2817 | 0.665 | −0.408 |
| NR | AACAGA | 1511.98 | 2383 | 1.576 | 0.455 |
| NR | AACCGC | 1370.77 | 1966 | 1.434 | 0.361 |
| NR | AACAGG | 1484.36 | 1903 | 1.282 | 0.248 |
| NR | AACCGA | 812.69 | 998 | 1.228 | 0.205 |
| NR | AACCGT | 586.88 | 706 | 1.203 | 0.185 |
| NR | AACCGG | 1499.21 | 1779 | 1.187 | 0.171 |
| NR | AATCGA | 737.89 | 687 | 0.931 | −0.071 |
| NR | AATCGT | 532.86 | 486 | 0.912 | −0.092 |
| NR | AATAGA | 1372.81 | 1117 | 0.814 | −0.206 |
| NR | AATCGC | 1244.60 | 602 | 0.484 | −0.726 |
| NR | AATAGG | 1347.73 | 643 | 0.477 | −0.740 |
| NR | AATCGG | 1361.22 | 593 | 0.436 | −0.831 |
| NS | AACAGC | 2917.73 | 4490 | 1.539 | 0.431 |
| NS | AACAGT | 1840.81 | 2414 | 1.311 | 0.271 |
| NS | AACTCG | 681.02 | 821 | 1.206 | 0.187 |
| NS | AATTCA | 1644.43 | 1970 | 1.198 | 0.181 |
| NS | AATTCT | 2034.08 | 2383 | 1.172 | 0.158 |
| NS | AACTCC | 2575.33 | 2818 | 1.094 | 0.090 |
| NS | AACTCA | 1811.14 | 1783 | 0.984 | −0.016 |
| NS | AACTCT | 2240.29 | 1981 | 0.884 | −0.123 |
| NS | AATAGT | 1671.38 | 1193 | 0.714 | −0.337 |
| NS | AATTCC | 2338.29 | 1655 | 0.708 | −0.346 |
| NS | AATAGC | 2649.17 | 1273 | 0.481 | −0.733 |
| NS | AATTCG | 618.33 | 241 | 0.390 | −0.942 |
| NT | AACACG | 860.22 | 1238 | 1.439 | 0.364 |
| NT | AACACA | 2119.90 | 2783 | 1.313 | 0.272 |
| NT | AACACC | 2618.65 | 3278 | 1.252 | 0.225 |
| NT | AACACT | 1874.68 | 2099 | 1.120 | 0.113 |
| NT | AATACT | 1702.13 | 1540 | 0.905 | −0.100 |
| NT | AATACA | 1924.77 | 1692 | 0.879 | −0.129 |
| NT | AATACC | 2377.62 | 1312 | 0.552 | −0.595 |
| NT | AATACG | 781.04 | 317 | 0.406 | −0.902 |
| NV | AATGTA | 927.15 | 1710 | 1.844 | 0.612 |
| NV | AATGTT | 1427.85 | 2573 | 1.802 | 0.589 |
| NV | AATGTC | 1828.10 | 2877 | 1.574 | 0.453 |
| NV | AATGTG | 3616.54 | 4314 | 1.193 | 0.176 |
| NV | AACGTG | 3983.18 | 2772 | 0.696 | −0.363 |
| NV | AACGTC | 2013.43 | 1341 | 0.666 | −0.406 |
| NV | AACGTT | 1572.60 | 509 | 0.324 | −1.128 |
| NV | AACGTA | 1021.14 | 294 | 0.288 | −1.245 |
| NW | AACTGG | 1808.22 | 2595 | 1.435 | 0.361 |
| NW | AATTGG | 1641.78 | 855 | 0.521 | −0.652 |
| NY | AACTAC | 2506.72 | 3191 | 1.273 | 0.241 |
| NY | AACTAT | 2036.89 | 2145 | 1.053 | 0.052 |
| NY | AATTAT | 1849.41 | 1795 | 0.971 | −0.030 |
| NY | AATTAC | 2275.98 | 1538 | 0.676 | −0.392 |
| PA | CCGGCG | 470.57 | 1166 | 2.478 | 0.907 |
| PA | CCGGCC | 1740.39 | 2666 | 1.532 | 0.426 |
| PA | CCAGCA | 2390.31 | 3368 | 1.409 | 0.343 |
| PA | CCAGCT | 2730.54 | 3622 | 1.326 | 0.283 |
| PA | CCTGCT | 2829.20 | 3750 | 1.325 | 0.282 |
| PA | CCTGCA | 2476.67 | 3178 | 1.283 | 0.249 |
| PA | CCAGCC | 4151.96 | 4942 | 1.190 | 0.174 |
| PA | CCCGCG | 1298.71 | 1528 | 1.177 | 0.163 |
| PA | CCTGCC | 4301.98 | 5000 | 1.162 | 0.150 |
| PA | CCAGCG | 1122.61 | 1078 | 0.960 | −0.041 |
| PA | CCTGCG | 1163.17 | 1105 | 0.950 | −0.051 |
| PA | CCGGCT | 1144.57 | 1013 | 0.885 | −0.122 |
| PA | CCGGCA | 1001.95 | 777 | 0.775 | −0.254 |
| PA | CCCGCC | 4803.25 | 2690 | 0.560 | −0.580 |
| PA | CCCGCA | 2765.26 | 846 | 0.306 | −1.184 |
| PA | CCCGCT | 3158.86 | 821 | 0.260 | −1.347 |
| PC | CCCTGC | 1550.51 | 2870 | 1.851 | 0.616 |
| PC | CCCTGT | 1305.97 | 1577 | 1.208 | 0.189 |
| PC | CCGTGC | 561.80 | 630 | 1.121 | 0.115 |
| PC | CCTTGT | 1169.67 | 1001 | 0.856 | −0.156 |
| PC | CCATGT | 1128.89 | 831 | 0.736 | −0.306 |
| PC | CCGTGT | 473.20 | 340 | 0.719 | −0.331 |
| PC | CCTTGC | 1388.69 | 937 | 0.675 | −0.393 |
| PC | CCATGC | 1340.27 | 733 | 0.547 | −0.603 |
| PD | CCAGAT | 2721.60 | 4165 | 1.530 | 0.425 |
| PD | CCTGAT | 2819.94 | 3781 | 1.341 | 0.293 |
| PD | CCGGAC | 1288.69 | 1659 | 1.287 | 0.253 |
| PD | CCAGAC | 3074.36 | 3766 | 1.225 | 0.203 |
| PD | CCTGAC | 3185.44 | 3646 | 1.145 | 0.135 |
| PD | CCGGAT | 1140.82 | 895 | 0.785 | −0.243 |
| PD | CCCGAC | 3556.62 | 2215 | 0.623 | −0.474 |
| PD | CCCGAT | 3148.53 | 809 | 0.257 | −1.359 |
| PE | CCAGAA | 3999.86 | 5699 | 1.425 | 0.354 |
| PE | CCTGAG | 5542.36 | 7122 | 1.285 | 0.251 |
| PE | CCGGAG | 2242.20 | 2870 | 1.280 | 0.247 |
| PE | CCAGAG | 5349.08 | 6777 | 1.267 | 0.237 |
| PE | CCTGAA | 4144.39 | 5108 | 1.233 | 0.209 |
| PE | CCCGAG | 6188.17 | 4149 | 0.670 | −0.400 |
| PE | CCGGAA | 1676.64 | 1032 | 0.616 | −0.485 |
| PE | CCCGAA | 4627.30 | 1013 | 0.219 | −1.519 |
| PF | CCCTTC | 2555.92 | 4301 | 1.683 | 0.520 |
| PF | CCATTT | 1930.27 | 2057 | 1.066 | 0.064 |
| PF | CCTTTT | 2000.01 | 1967 | 0.983 | −0.017 |
| PF | CCCTTT | 2233.06 | 2159 | 0.967 | −0.034 |
| PF | CCTTTC | 2289.18 | 2078 | 0.908 | −0.097 |
| PF | CCGTTC | 926.10 | 662 | 0.715 | −0.336 |
| PF | CCATTC | 2209.35 | 1290 | 0.584 | −0.538 |
| PF | CCGTTT | 809.12 | 439 | 0.543 | −0.611 |
| PG | CCTGGG | 2918.52 | 4310 | 1.477 | 0.390 |
| PG | CCTGGA | 2989.52 | 4317 | 1.444 | 0.367 |
| PG | CCGGGC | 1639.82 | 2353 | 1.435 | 0.361 |
| PG | CCGGGG | 1180.71 | 1657 | 1.403 | 0.339 |
| PG | CCTGGT | 1935.48 | 2673 | 1.381 | 0.323 |
| PG | CCAGGA | 2885.27 | 3897 | 1.351 | 0.301 |
| PG | CCAGGG | 2816.75 | 3472 | 1.233 | 0.209 |
| PG | CCAGGT | 1867.98 | 2259 | 1.209 | 0.190 |
| PG | CCTGGC | 4053.37 | 4622 | 1.140 | 0.131 |
| PG | CCAGGC | 3912.02 | 4106 | 1.050 | 0.048 |
| PG | CCGGGT | 783.01 | 661 | 0.844 | −0.169 |
| PG | CCGGGA | 1209.43 | 963 | 0.796 | −0.228 |
| PG | CCCGGG | 3258.60 | 2136 | 0.655 | −0.422 |
| PG | CCCGGC | 4525.68 | 2555 | 0.565 | −0.572 |
| PG | CCCGGA | 3337.86 | 968 | 0.290 | −1.238 |
| PG | CCCGGT | 2161.00 | 526 | 0.243 | −1.413 |
| PH | CCGCAC | 725.13 | 972 | 1.340 | 0.293 |
| PH | CCCCAC | 2001.25 | 2505 | 1.252 | 0.225 |
| PH | CCTCAT | 1299.79 | 1592 | 1.225 | 0.203 |
| PH | CCACAT | 1254.46 | 1222 | 0.974 | −0.026 |
| PH | CCCCAT | 1451.24 | 1303 | 0.898 | −0.108 |
| PH | CCTCAC | 1792.40 | 1531 | 0.854 | −0.158 |
| PH | CCACAC | 1729.89 | 1366 | 0.790 | −0.236 |
| PH | CCGCAT | 525.84 | 289 | 0.550 | −0.599 |
| PI | CCCATC | 2119.04 | 4651 | 2.195 | 0.786 |
| PI | CCCATT | 1675.71 | 2102 | 1.254 | 0.227 |
| PI | CCAATA | 666.18 | 819 | 1.229 | 0.207 |
| PI | CCCATA | 770.68 | 776 | 1.007 | 0.007 |
| PI | CCAATT | 1448.49 | 1386 | 0.957 | −0.044 |
| PI | CCTATA | 690.25 | 603 | 0.874 | −0.135 |
| PI | CCTATT | 1500.83 | 1266 | 0.844 | −0.170 |
| PI | CCAATC | 1831.71 | 939 | 0.513 | −0.668 |
| PI | CCTATC | 1897.89 | 957 | 0.504 | −0.685 |
| PI | CCGATT | 607.17 | 299 | 0.492 | −0.708 |
| PI | CCGATC | 767.80 | 342 | 0.445 | −0.809 |
| PI | CCGATA | 279.24 | 115 | 0.412 | −0.887 |
| PK | CCCAAG | 3738.47 | 6383 | 1.707 | 0.535 |
| PK | CCCAAA | 2886.60 | 3787 | 1.312 | 0.271 |
| PK | CCAAAA | 2495.20 | 2489 | 0.998 | −0.002 |
| PK | CCAAAG | 3231.55 | 3127 | 0.968 | −0.033 |
| PK | CCTAAA | 2585.35 | 1840 | 0.712 | −0.340 |
| PK | CCGAAG | 1354.58 | 940 | 0.694 | −0.365 |
| PK | CCTAAG | 3348.32 | 1660 | 0.496 | −0.702 |
| PK | CCGAAA | 1045.92 | 460 | 0.440 | −0.821 |
| PL | CCGCTG | 1824.84 | 3343 | 1.832 | 0.605 |
| PL | CCGCTC | 878.12 | 1254 | 1.428 | 0.356 |
| PL | CCTTTG | 1466.52 | 2054 | 1.401 | 0.337 |
| PL | CCTTTA | 872.97 | 1195 | 1.369 | 0.314 |
| PL | CCCTTG | 1637.40 | 2122 | 1.296 | 0.259 |
| PL | CCTCTT | 1496.70 | 1827 | 1.221 | 0.199 |
| PL | CCCCTG | 5036.31 | 5760 | 1.144 | 0.134 |
| PL | CCCCTC | 2423.49 | 2646 | 1.092 | 0.088 |
| PL | CCTCTA | 807.16 | 871 | 1.079 | 0.076 |
| PL | CCATTA | 842.53 | 826 | 0.980 | −0.020 |
| PL | CCACTT | 1444.51 | 1371 | 0.949 | −0.052 |
| PL | CCACTA | 779.01 | 729 | 0.936 | −0.066 |
| PL | CCTCTC | 2170.57 | 1934 | 0.891 | −0.115 |
| PL | CCTCTG | 4510.71 | 3745 | 0.830 | −0.186 |
| PL | CCATTG | 1415.38 | 1172 | 0.828 | −0.189 |
| PL | CCCCTT | 1671.10 | 1324 | 0.792 | −0.233 |
| PL | CCGCTA | 326.54 | 255 | 0.781 | −0.247 |
| PL | CCCCTA | 901.21 | 689 | 0.765 | −0.268 |
| PL | CCACTG | 4353.41 | 3218 | 0.739 | −0.302 |
| PL | CCCTTA | 974.69 | 709 | 0.727 | −0.318 |
| PL | CCACTC | 2094.88 | 1475 | 0.704 | −0.351 |
| PL | CCGTTG | 593.29 | 402 | 0.678 | −0.389 |
| PL | CCGCTT | 605.50 | 402 | 0.664 | −0.410 |
| PL | CCGTTA | 353.17 | 157 | 0.445 | −0.811 |
| PM | CCCATG | 2307.54 | 3923 | 1.700 | 0.531 |
| PM | CCAATG | 1994.65 | 1552 | 0.778 | −0.251 |
| PM | CCGATG | 836.10 | 520 | 0.622 | −0.475 |
| PM | CCTATG | 2066.72 | 1210 | 0.585 | −0.535 |
| PN | CCCAAC | 2313.61 | 4255 | 1.839 | 0.609 |
| PN | CCAAAT | 1815.81 | 2453 | 1.351 | 0.301 |
| PN | CCCAAT | 2100.65 | 2296 | 1.093 | 0.089 |
| PN | CCAAAC | 1999.90 | 1735 | 0.868 | −0.142 |
| PN | CCTAAT | 1881.42 | 1342 | 0.713 | −0.338 |
| PN | CCTAAC | 2072.16 | 997 | 0.481 | −0.732 |
| PN | CCGAAT | 761.14 | 340 | 0.447 | −0.806 |
| PN | CCGAAC | 838.30 | 365 | 0.435 | −0.831 |
| PP | CCGCCG | 608.57 | 2335 | 3.837 | 1.345 |
| PP | CCGCCC | 1679.58 | 2697 | 1.606 | 0.474 |
| PP | CCCCCG | 1679.58 | 2420 | 1.441 | 0.365 |
| PP | CCTCCA | 3588.72 | 4314 | 1.202 | 0.184 |
| PP | CCTCCT | 3718.39 | 4305 | 1.158 | 0.146 |
| PP | CCACCA | 3463.58 | 3850 | 1.112 | 0.106 |
| PP | CCACCT | 3588.72 | 3798 | 1.058 | 0.057 |
| PP | CCCCCA | 4006.89 | 4095 | 1.022 | 0.022 |
| PP | CCACCC | 4006.89 | 3595 | 0.897 | −0.108 |
| PP | CCGCCA | 1451.84 | 1280 | 0.882 | −0.126 |
| PP | CCACCG | 1451.84 | 1252 | 0.862 | −0.148 |
| PP | CCGCCT | 1504.30 | 1286 | 0.855 | −0.157 |
| PP | CCTCCC | 4151.67 | 3338 | 0.804 | −0.218 |
| PP | CCTCCG | 1504.30 | 1152 | 0.766 | −0.267 |
| PP | CCCCCT | 4151.67 | 3160 | 0.761 | −0.273 |
| PP | CCCCCC | 4635.43 | 2315 | 0.499 | −0.694 |
| PQ | CCCCAG | 5063.98 | 6421 | 1.268 | 0.237 |
| PQ | CCGCAG | 1834.86 | 2187 | 1.192 | 0.176 |
| PQ | CCTCAA | 1624.21 | 1752 | 1.079 | 0.076 |
| PQ | CCTCAG | 4535.49 | 4221 | 0.931 | −0.072 |
| PQ | CCACAA | 1567.57 | 1405 | 0.896 | −0.109 |
| PQ | CCACAG | 4377.33 | 3670 | 0.838 | −0.176 |
| PQ | CCCCAA | 1813.47 | 1497 | 0.825 | −0.192 |
| PQ | CCGCAA | 657.08 | 321 | 0.489 | −0.716 |
| PR | CCGCGC | 563.43 | 1094 | 1.942 | 0.664 |
| PR | CCGCGG | 616.23 | 1113 | 1.806 | 0.591 |
| PR | CCCAGG | 1683.86 | 2927 | 1.738 | 0.553 |
| PR | CCCCGG | 1700.71 | 2608 | 1.533 | 0.428 |
| PR | CCCCGC | 1555.00 | 1979 | 1.273 | 0.241 |
| PR | CCCCGA | 921.92 | 1166 | 1.265 | 0.235 |
| PR | CCTCGA | 825.71 | 1015 | 1.229 | 0.206 |
| PR | CCAAGA | 1482.62 | 1608 | 1.085 | 0.081 |
| PR | CCTCGT | 596.27 | 644 | 1.080 | 0.077 |
| PR | CCCAGA | 1715.19 | 1801 | 1.050 | 0.049 |
| PR | CCGAGG | 610.12 | 636 | 1.042 | 0.042 |
| PR | CCTCGG | 1523.22 | 1511 | 0.992 | −0.008 |
| PR | CCCCGT | 665.75 | 655 | 0.984 | −0.016 |
| PR | CCAAGG | 1455.54 | 1347 | 0.925 | −0.077 |
| PR | CCACGA | 796.91 | 632 | 0.793 | −0.232 |
| PR | CCGCGT | 241.23 | 191 | 0.792 | −0.233 |
| PR | CCACGT | 575.48 | 418 | 0.726 | −0.320 |
| PR | CCACGG | 1470.10 | 1040 | 0.707 | −0.346 |
| PR | CCGCGA | 334.04 | 226 | 0.677 | −0.391 |
| PR | CCTCGC | 1392.72 | 838 | 0.602 | −0.508 |
| PR | CCACGC | 1344.15 | 701 | 0.522 | −0.651 |
| PR | CCGAGA | 621.48 | 308 | 0.496 | −0.702 |
| PR | CCTAGA | 1536.19 | 692 | 0.450 | −0.797 |
| PR | CCTAGG | 1508.13 | 586 | 0.389 | −0.945 |
| PS | CCCAGC | 3196.25 | 6398 | 2.002 | 0.694 |
| PS | CCCTCG | 746.03 | 1385 | 1.856 | 0.619 |
| PS | CCGTCG | 270.31 | 483 | 1.787 | 0.580 |
| PS | CCCAGT | 2016.53 | 2743 | 1.360 | 0.308 |
| PS | CCTTCA | 1776.97 | 2263 | 1.274 | 0.242 |
| PS | CCTTCT | 2198.02 | 2711 | 1.233 | 0.210 |
| PS | CCCTCC | 2821.16 | 3353 | 1.189 | 0.173 |
| PS | CCATCA | 1715.00 | 1819 | 1.061 | 0.059 |
| PS | CCATCT | 2121.37 | 2183 | 1.029 | 0.029 |
| PS | CCTTCC | 2526.74 | 2594 | 1.027 | 0.026 |
| PS | CCGTCC | 1022.21 | 1048 | 1.025 | 0.025 |
| PS | CCCTCA | 1984.02 | 1945 | 0.980 | −0.020 |
| PS | CCAAGT | 1743.10 | 1582 | 0.908 | −0.097 |
| PS | CCCTCT | 2454.14 | 2113 | 0.861 | −0.150 |
| PS | CCTTCG | 668.17 | 552 | 0.826 | −0.191 |
| PS | CCATCC | 2438.63 | 1995 | 0.818 | −0.201 |
| PS | CCGAGC | 1158.11 | 885 | 0.764 | −0.269 |
| PS | CCATCG | 644.87 | 475 | 0.737 | −0.306 |
| PS | CCAAGC | 2762.85 | 1659 | 0.600 | −0.510 |
| PS | CCGTCT | 889.22 | 523 | 0.588 | −0.531 |
| PS | CCGAGT | 730.66 | 371 | 0.508 | −0.678 |
| PS | CCGTCA | 718.88 | 364 | 0.506 | −0.681 |
| PS | CCTAGT | 1806.08 | 860 | 0.476 | −0.742 |
| PS | CCTAGC | 2862.68 | 968 | 0.338 | −1.084 |
| PT | CCCACG | 829.55 | 1764 | 2.126 | 0.754 |
| PT | CCCACC | 2525.29 | 4586 | 1.816 | 0.597 |
| PT | CCCACA | 2044.32 | 2719 | 1.330 | 0.285 |
| PT | CCCACT | 1807.85 | 2282 | 1.262 | 0.233 |
| PT | CCAACA | 1767.12 | 1895 | 1.072 | 0.070 |
| PT | CCAACT | 1562.71 | 1593 | 1.019 | 0.019 |
| PT | CCGACG | 300.57 | 305 | 1.015 | 0.015 |
| PT | CCTACT | 1619.18 | 1252 | 0.773 | −0.257 |
| PT | CCAACC | 2182.87 | 1514 | 0.694 | −0.366 |
| PT | CCTACA | 1830.97 | 1241 | 0.678 | −0.389 |
| PT | CCGACC | 915.00 | 592 | 0.647 | −0.435 |
| PT | CCAACG | 717.06 | 463 | 0.646 | −0.437 |
| PT | CCTACC | 2261.75 | 1251 | 0.553 | −0.592 |
| PT | CCGACT | 655.05 | 342 | 0.522 | −0.650 |
| PT | CCGACA | 740.73 | 352 | 0.475 | −0.744 |
| PT | CCTACG | 742.97 | 352 | 0.474 | −0.747 |
| PV | CCTGTT | 1493.79 | 2375 | 1.590 | 0.464 |
| PV | CCTGTA | 969.97 | 1482 | 1.528 | 0.424 |
| PV | CCAGTA | 936.15 | 1352 | 1.444 | 0.368 |
| PV | CCTGTG | 3783.57 | 5362 | 1.417 | 0.349 |
| PV | CCAGTT | 1441.70 | 2038 | 1.414 | 0.346 |
| PV | CCTGTC | 1912.53 | 2666 | 1.394 | 0.332 |
| PV | CCGGTG | 1530.67 | 1911 | 1.248 | 0.222 |
| PV | CCAGTG | 3651.63 | 3787 | 1.037 | 0.036 |
| PV | CCAGTC | 1845.84 | 1863 | 1.009 | 0.009 |
| PV | CCGGTC | 773.73 | 778 | 1.006 | 0.006 |
| PV | CCCGTG | 4224.44 | 2576 | 0.610 | −0.495 |
| PV | CCGGTT | 604.32 | 351 | 0.581 | −0.543 |
| PV | CCGGTA | 392.41 | 215 | 0.548 | −0.602 |
| PV | CCCGTC | 2135.39 | 1084 | 0.508 | −0.678 |
| PV | CCCGTT | 1667.85 | 391 | 0.234 | −1.451 |
| PV | CCCGTA | 1083.00 | 216 | 0.199 | −1.612 |
| PW | CCCTGG | 1769.80 | 2753 | 1.556 | 0.442 |
| PW | CCGTGG | 641.26 | 661 | 1.031 | 0.030 |
| PW | CCATGG | 1529.83 | 1060 | 0.693 | −0.367 |
| PW | CCTTGG | 1585.10 | 1052 | 0.664 | −0.410 |
| PY | CCCTAC | 2166.25 | 3378 | 1.559 | 0.444 |
| PY | CCCTAT | 1760.24 | 2097 | 1.191 | 0.175 |
| PY | CCTTAT | 1576.54 | 1702 | 1.080 | 0.077 |
| PY | CCATAT | 1521.56 | 1513 | 0.994 | −0.006 |
| PY | CCTTAC | 1940.18 | 1485 | 0.765 | −0.267 |
| PY | CCGTAC | 784.91 | 592 | 0.754 | −0.282 |
| PY | CCGTAT | 637.80 | 429 | 0.673 | −0.397 |
| PY | CCATAC | 1872.52 | 1064 | 0.568 | −0.565 |
| QA | CAAGCA | 1597.87 | 2339 | 1.464 | 0.381 |
| QA | CAAGCT | 1825.31 | 2409 | 1.320 | 0.277 |
| QA | CAGGCG | 2095.55 | 2271 | 1.084 | 0.080 |
| QA | CAGGCC | 7750.37 | 7695 | 0.993 | −0.007 |
| QA | CAAGCC | 2775.49 | 2655 | 0.957 | −0.044 |
| QA | CAGGCT | 5097.04 | 4584 | 0.899 | −0.106 |
| QA | CAGGCA | 4461.94 | 3943 | 0.884 | −0.124 |
| QA | CAAGCG | 750.44 | 458 | 0.610 | −0.494 |
| QC | CAGTGT | 2490.13 | 2791 | 1.121 | 0.114 |
| QC | CAGTGC | 2956.40 | 3260 | 1.103 | 0.098 |
| QC | CAATGT | 891.74 | 822 | 0.922 | −0.081 |
| QC | CAATGC | 1058.72 | 524 | 0.495 | −0.703 |
| QD | CAAGAT | 2128.42 | 3326 | 1.563 | 0.446 |
| QD | CAAGAC | 2404.29 | 2506 | 1.042 | 0.041 |
| QD | CAGGGC | 6713.82 | 6642 | 0.989 | −0.011 |
| QD | CAGGAT | 5943.46 | 4716 | 0.793 | −0.231 |
| QE | CAAGAA | 3247.03 | 5286 | 1.628 | 0.487 |
| QE | CAGGAG | 12125.58 | 12556 | 1.035 | 0.035 |
| QE | CAAGAG | 4342.30 | 4206 | 0.969 | −0.032 |
| QE | CAGGAA | 9067.09 | 6734 | 0.743 | −0.297 |
| QF | CAGTTT | 3509.26 | 4032 | 1.149 | 0.139 |
| QF | CAGTTC | 4016.64 | 4205 | 1.047 | 0.046 |
| QF | CAATTT | 1256.70 | 1156 | 0.920 | −0.084 |
| QF | CAATTC | 1438.40 | 828 | 0.576 | −0.552 |
| QG | CAAGGA | 1440.03 | 2837 | 1.970 | 0.678 |
| QG | CAGGGT | 932.30 | 1506 | 1.615 | 0.480 |
| QG | CAGGGG | 1405.83 | 1700 | 1.209 | 0.190 |
| QG | CAGGGC | 1952.47 | 2192 | 1.123 | 0.116 |
| QG | CAGGGC | 5452.14 | 5605 | 1.028 | 0.028 |
| QG | CAGGGT | 2603.39 | 2292 | 0.880 | −0.127 |
| QG | CAGGGA | 4021.17 | 2871 | 0.714 | −0.337 |
| QG | CAGGGG | 3925.67 | 2730 | 0.695 | −0.363 |
| QH | CAACAT | 1067.82 | 1364 | 1.277 | 0.245 |
| QH | CAGCAC | 4111.88 | 4483 | 1.090 | 0.086 |
| QH | CAGCAT | 2981.80 | 2794 | 0.937 | −0.065 |
| QH | CAACAC | 1472.51 | 993 | 0.674 | −0.394 |
| QI | CAAATA | 656.37 | 1125 | 1.714 | 0.539 |
| QI | CAAATT | 1427.17 | 1667 | 1.168 | 0.155 |
| QI | CAGATC | 5039.60 | 5197 | 1.031 | 0.031 |
| QI | CAGATA | 1832.87 | 1802 | 0.983 | −0.017 |
| QI | CAGATT | 3985.26 | 3693 | 0.927 | −0.076 |
| QI | CAAATC | 1804.74 | 1262 | 0.699 | −0.358 |
| QK | CAGGAG | 8990.94 | 9726 | 1.082 | 0.079 |
| QK | CAAGAA | 2486.09 | 2610 | 1.050 | 0.049 |
| QK | CAGGAA | 6942.22 | 6532 | 0.941 | −0.061 |
| QK | CAAGAG | 3219.76 | 2771 | 0.861 | −0.150 |
| QL | CAGCTG | 10304.18 | 12629 | 1.226 | 0.203 |
| QL | CAACTA | 660.31 | 798 | 1.209 | 0.189 |
| QL | CAACTT | 1224.39 | 1479 | 1.208 | 0.189 |
| QL | CAGCTC | 4958.40 | 5986 | 1.207 | 0.188 |
| QL | CAGCTA | 1843.86 | 2002 | 1.086 | 0.082 |
| QL | CAGCTT | 3419.03 | 3476 | 1.017 | 0.017 |
| QL | CAATTA | 714.15 | 642 | 0.899 | −0.107 |
| QL | CAGTTG | 3350.09 | 2597 | 0.775 | −0.255 |
| QL | CAGTTA | 1994.20 | 1518 | 0.761 | −0.273 |
| QL | CAACTC | 1775.66 | 1279 | 0.720 | −0.328 |
| QL | CAACTG | 3690.04 | 2093 | 0.567 | −0.567 |
| QL | CAATTG | 1199.70 | 635 | 0.529 | −0.636 |
| QM | CAGATG | 5587.91 | 5592 | 1.001 | 0.001 |
| QM | CAAATG | 2001.09 | 1997 | 0.998 | −0.002 |
| QN | CAAAAT | 1720.47 | 2394 | 1.391 | 0.330 |
| QN | CAGAGC | 5291.34 | 5195 | 0.982 | −0.018 |
| QN | CAGAAT | 4804.30 | 4430 | 0.922 | −0.081 |
| QN | CAAAGC | 1894.89 | 1692 | 0.893 | −0.113 |
| QP | CAGCCG | 1816.66 | 2237 | 1.231 | 0.208 |
| QP | CAGCCC | 5013.75 | 6143 | 1.225 | 0.203 |
| QP | CAGCCT | 4490.51 | 4526 | 1.008 | 0.008 |
| QP | CAGCCA | 4333.91 | 4235 | 0.977 | −0.023 |
| QP | CAACCA | 1552.02 | 1441 | 0.928 | −0.074 |
| QP | CAACCT | 1608.10 | 1304 | 0.811 | −0.210 |
| QP | CAACCC | 1795.48 | 1132 | 0.630 | −0.461 |
| QP | CAACCG | 650.57 | 243 | 0.374 | −0.985 |
| QQ | CAACCA | 1545.49 | 1866 | 1.207 | 0.188 |
| QQ | CAGCAG | 12051.19 | 13131 | 1.090 | 0.086 |
| QQ | CAGCCA | 4315.66 | 4034 | 0.935 | −0.067 |
| QQ | CAACAG | 4315.66 | 3197 | 0.741 | −0.300 |
| QR | CAAAGA | 1214.45 | 1863 | 1.534 | 0.428 |
| QR | CAGAGG | 3329.32 | 4331 | 1.301 | 0.263 |
| QR | CAAAGG | 1192.27 | 1360 | 1.141 | 0.132 |
| QR | CAGAGA | 3391.27 | 3777 | 1.114 | 0.108 |
| QR | CAGCGC | 3074.54 | 3169 | 1.031 | 0.030 |
| QR | CAGCGG | 3362.63 | 3352 | 0.997 | −0.003 |
| QR | CAGCGT | 1316.32 | 1215 | 0.923 | −0.080 |
| QR | CAGCGA | 1822.82 | 1469 | 0.806 | −0.216 |
| QR | CAACGT | 471.39 | 327 | 0.694 | −0.366 |
| QR | CAACGA | 652.77 | 413 | 0.633 | −0.458 |
| QR | CAACGG | 1204.20 | 453 | 0.376 | −0.978 |
| QR | CAACGC | 1101.03 | 404 | 0.367 | −1.003 |
| QS | CAAAGT | 904.91 | 1408 | 1.556 | 0.442 |
| QS | CAGAGC | 4005.17 | 5248 | 1.310 | 0.270 |
| QS | CAGAGT | 2526.89 | 2963 | 1.173 | 0.159 |
| QS | CAAAGC | 1434.30 | 1465 | 1.021 | 0.021 |
| QS | CAGTCG | 934.84 | 923 | 0.987 | −0.013 |
| QS | CAGTCA | 2486.15 | 2379 | 0.957 | −0.044 |
| QS | CAGTCT | 3075.24 | 2806 | 0.912 | −0.092 |
| QS | CAATCA | 890.32 | 781 | 0.877 | −0.131 |
| QS | CAGTCC | 3535.16 | 3051 | 0.863 | −0.147 |
| QS | CAATCT | 1101.28 | 765 | 0.695 | −0.364 |
| QS | CAATCC | 1265.98 | 587 | 0.464 | −0.769 |
| QS | CAATCG | 334.78 | 119 | 0.355 | −1.034 |
| QT | CAAACT | 1116.05 | 1463 | 1.311 | 0.271 |
| QT | CAAACA | 1262.03 | 1602 | 1.269 | 0.239 |
| QT | CAGACG | 1430.02 | 1665 | 1.164 | 0.152 |
| QT | CAGACC | 4353.25 | 4301 | 0.988 | −0.012 |
| QT | CAGACA | 3524.12 | 3445 | 0.978 | −0.023 |
| QT | CAGACT | 3116.48 | 2792 | 0.896 | −0.110 |
| QT | CAAACC | 1558.95 | 1232 | 0.790 | −0.235 |
| QT | CAAACG | 512.11 | 373 | 0.728 | −0.317 |
| QV | CAAGTA | 657.01 | 1210 | 1.842 | 0.611 |
| QV | CAAGTT | 1011.82 | 1737 | 1.717 | 0.540 |
| QV | CAAGTC | 1295.45 | 1468 | 1.133 | 0.125 |
| QV | CAAGTG | 2562.79 | 2712 | 1.058 | 0.057 |
| QV | CAGGTG | 7156.41 | 7062 | 0.987 | −0.013 |
| QV | CAGGTC | 3617.45 | 3213 | 0.888 | −0.119 |
| QV | CAGGTT | 2825.43 | 2269 | 0.803 | −0.219 |
| QV | CAGGTA | 1834.65 | 1290 | 0.703 | −0.352 |
| QW | CAGTGG | 3057.92 | 3447 | 1.127 | 0.120 |
| QW | CAATGG | 1095.08 | 706 | 0.645 | −0.439 |
| QY | CAATAT | 1029.01 | 1120 | 1.088 | 0.085 |
| QY | CAGTAC | 3536.21 | 3820 | 1.080 | 0.077 |
| QY | CAGTAT | 2873.43 | 2979 | 1.037 | 0.036 |
| QY | CAATAC | 1266.36 | 786 | 0.621 | −0.477 |
| RA | CGGGCG | 659.18 | 1185 | 1.798 | 0.587 |
| RA | CGGGCC | 2437.97 | 3513 | 1.441 | 0.365 |
| RA | AGAGCA | 1415.51 | 1970 | 1.392 | 0.331 |
| RA | CGCGCG | 602.71 | 827 | 1.372 | 0.316 |
| RA | CGTGCC | 954.35 | 1266 | 1.327 | 0.283 |
| RA | CGAGCA | 760.84 | 970 | 1.275 | 0.243 |
| RA | CGAGCT | 869.13 | 1108 | 1.275 | 0.243 |
| RA | CGAGCC | 1321.57 | 1595 | 1.207 | 0.188 |
| RA | AGAGCT | 1616.99 | 1949 | 1.205 | 0.187 |
| RA | CGTGCT | 627.63 | 744 | 1.185 | 0.170 |
| RA | CGGGCA | 1403.55 | 1612 | 1.149 | 0.138 |
| RA | CGTGCA | 549.43 | 570 | 1.037 | 0.037 |
| RA | CGTGCG | 258.04 | 250 | 0.969 | −0.032 |
| RA | CGAGCG | 357.33 | 341 | 0.954 | −0.047 |
| RA | AGGGCC | 2413.81 | 2173 | 0.900 | −0.105 |
| RA | AGAGCC | 2458.73 | 2202 | 0.896 | −0.110 |
| RA | CGGGCT | 1603.33 | 1435 | 0.895 | −0.111 |
| RA | AGGGCA | 1389.65 | 1242 | 0.894 | −0.112 |
| RA | AGGGCT | 1587.45 | 1311 | 0.826 | −0.191 |
| RA | AGGGCG | 652.65 | 524 | 0.803 | −0.220 |
| RA | CGCGCC | 2229.09 | 1712 | 0.768 | −0.264 |
| RA | AGAGCG | 664.79 | 384 | 0.578 | −0.549 |
| RA | CGCGCA | 1283.30 | 331 | 0.258 | −1.355 |
| RA | CGCGCT | 1465.97 | 369 | 0.252 | −1.379 |
| RC | CGCTGC | 986.26 | 2873 | 2.913 | 1.069 |
| RC | CGCTGT | 830.71 | 1313 | 1.581 | 0.458 |
| RC | CGTTGT | 355.66 | 320 | 0.900 | −0.106 |
| RC | CGTTGC | 422.25 | 372 | 0.881 | −0.127 |
| RC | AGATGT | 916.29 | 806 | 0.880 | −0.128 |
| RC | CGATGT | 492.51 | 421 | 0.855 | −0.157 |
| RC | AGGTGT | 899.55 | 671 | 0.746 | −0.293 |
| RC | AGGTGC | 1067.99 | 758 | 0.710 | −0.343 |
| RC | CGATGC | 584.73 | 381 | 0.652 | −0.428 |
| RC | CGGTGC | 1078.67 | 660 | 0.612 | −0.491 |
| RC | AGATGC | 1087.86 | 642 | 0.590 | −0.527 |
| RC | CGGTGT | 908.55 | 414 | 0.456 | −0.786 |
| RD | AGAGAT | 2027.66 | 2952 | 1.456 | 0.376 |
| RD | CGGGAC | 2271.13 | 3231 | 1.423 | 0.353 |
| RD | CGAGAT | 1089.87 | 1500 | 1.376 | 0.319 |
| RD | CGAGAC | 1231.14 | 1693 | 1.375 | 0.319 |
| RD | CGTGAC | 889.05 | 1044 | 1.174 | 0.161 |
| RD | AGAGAC | 2290.48 | 2433 | 1.062 | 0.060 |
| RD | CGTGAT | 787.04 | 833 | 1.058 | 0.057 |
| RD | AGGGAC | 2248.63 | 2322 | 1.033 | 0.032 |
| RD | AGGGAT | 1990.62 | 1732 | 0.870 | −0.139 |
| RD | CGGGAT | 2010.54 | 1606 | 0.799 | −0.225 |
| RD | CGCGAC | 2076.56 | 1092 | 0.526 | −0.643 |
| RD | CGCGAT | 1838.29 | 313 | 0.170 | −1.770 |
| RE | AGAGAA | 2644.21 | 4195 | 1.586 | 0.462 |
| RE | CGGGAG | 3506.29 | 5344 | 1.524 | 0.421 |
| RE | CGAGAG | 1900.69 | 2475 | 1.302 | 0.264 |
| RE | CGAGAA | 1421.27 | 1844 | 1.297 | 0.260 |
| RE | CGTGAG | 1372.55 | 1453 | 1.059 | 0.057 |
| RE | AGGGAG | 3471.55 | 3469 | 0.999 | −0.001 |
| RE | AGAGAG | 3536.15 | 3392 | 0.959 | −0.042 |
| RE | CGTGAA | 1026.35 | 947 | 0.923 | −0.080 |
| RE | AGGGAA | 2595.91 | 2343 | 0.903 | −0.103 |
| RE | CGGGAA | 2621.88 | 2131 | 0.813 | −0.207 |
| RE | CGCGAG | 3205.89 | 1839 | 0.574 | −0.556 |
| RE | CGCGAA | 2397.25 | 268 | 0.112 | −2.191 |
| RF | CGCTTC | 1446.49 | 3411 | 2.358 | 0.858 |
| RF | CGTTTC | 619.29 | 823 | 1.329 | 0.284 |
| RF | CGTTTT | 541.07 | 705 | 1.303 | 0.265 |
| RF | AGATTT | 1393.96 | 1531 | 1.098 | 0.094 |
| RF | CGCTTT | 1263.77 | 1366 | 1.081 | 0.078 |
| RF | CGATTT | 749.26 | 772 | 1.030 | 0.030 |
| RF | AGGTTT | 1368.50 | 1295 | 0.946 | −0.055 |
| RF | AGGTTC | 1566.36 | 1192 | 0.761 | −0.273 |
| RF | CGATTC | 857.59 | 632 | 0.737 | −0.305 |
| RF | CGGTTC | 1582.03 | 951 | 0.601 | −0.509 |
| RF | AGATTC | 1595.50 | 944 | 0.592 | −0.525 |
| RF | CGGTTT | 1382.19 | 744 | 0.538 | −0.619 |
| RG | CGTGGT | 370.38 | 685 | 1.849 | 0.615 |
| RG | CGTGGG | 558.50 | 980 | 1.755 | 0.562 |
| RG | CGTGGC | 775.66 | 1315 | 1.695 | 0.528 |
| RG | CGAGGA | 792.21 | 1266 | 1.598 | 0.469 |
| RG | CGAGGG | 773.39 | 1219 | 1.576 | 0.455 |
| RG | AGAGGA | 1473.87 | 2281 | 1.548 | 0.437 |
| RG | CGAGGT | 512.89 | 789 | 1.538 | 0.431 |
| RG | CGGGGC | 1981.48 | 2952 | 1.490 | 0.399 |
| RG | CGTGGA | 572.08 | 844 | 1.475 | 0.389 |
| RG | CGAGGC | 1074.12 | 1569 | 1.461 | 0.379 |
| RG | AGAGGT | 954.21 | 1128 | 1.182 | 0.167 |
| RG | CGGGGT | 946.15 | 918 | 0.970 | −0.030 |
| RG | CGCGGC | 1811.72 | 1574 | 0.869 | −0.141 |
| RG | AGGGGC | 1961.86 | 1660 | 0.846 | −0.167 |
| RG | AGAGGC | 1998.36 | 1680 | 0.841 | −0.174 |
| RG | AGAGGG | 1438.87 | 1203 | 0.836 | −0.179 |
| RG | AGGGGT | 936.78 | 777 | 0.829 | −0.187 |
| RG | CGGGGG | 1426.72 | 1146 | 0.803 | −0.219 |
| RG | CGGGGA | 1461.42 | 1140 | 0.780 | −0.248 |
| RG | CGCGGG | 1304.48 | 904 | 0.693 | −0.367 |
| RG | AGGGGA | 1446.94 | 923 | 0.638 | −0.450 |
| RG | AGGGGG | 1412.58 | 683 | 0.484 | −0.727 |
| RG | CGCGGT | 865.09 | 248 | 0.287 | −1.249 |
| RG | CGCGGA | 1336.22 | 302 | 0.226 | −1.487 |
| RH | CGCCAC | 1288.00 | 1861 | 1.445 | 0.368 |
| RH | CGGCAC | 1408.69 | 1707 | 1.212 | 0.192 |
| RH | AGACAT | 1030.24 | 1201 | 1.166 | 0.153 |
| RH | CGTCAT | 399.89 | 447 | 1.118 | 0.111 |
| RH | AGGCAT | 1011.41 | 988 | 0.977 | −0.023 |
| RH | CGACAT | 553.75 | 530 | 0.957 | −0.044 |
| RH | AGGCAC | 1394.73 | 1292 | 0.926 | −0.077 |
| RH | AGACAC | 1420.69 | 1212 | 0.853 | −0.159 |
| RH | CGTCAC | 551.44 | 468 | 0.849 | −0.164 |
| RH | CGACAC | 763.62 | 614 | 0.804 | −0.218 |
| RH | CGCCAT | 934.02 | 728 | 0.779 | −0.249 |
| RH | CGGCAT | 1021.53 | 730 | 0.715 | −0.336 |
| RI | CGCATC | 1625.56 | 2948 | 1.814 | 0.595 |
| RI | AGAATA | 652.11 | 1175 | 1.802 | 0.589 |
| RI | AGAATT | 1417.90 | 2185 | 1.541 | 0.432 |
| RI | AGGATA | 640.20 | 804 | 1.256 | 0.228 |
| RI | CGAATA | 350.51 | 439 | 1.252 | 0.225 |
| RI | CGAATT | 762.13 | 850 | 1.115 | 0.109 |
| RI | AGGATT | 1392.00 | 1366 | 0.981 | −0.019 |
| RI | AGGATC | 1760.27 | 1662 | 0.944 | −0.057 |
| RI | CGAATC | 963.75 | 802 | 0.832 | −0.184 |
| RI | CGGATC | 1777.88 | 1479 | 0.832 | −0.184 |
| RI | AGAATC | 1793.03 | 1389 | 0.775 | −0.255 |
| RI | CGTATT | 550.36 | 408 | 0.741 | −0.299 |
| RI | CGCATT | 1285.48 | 913 | 0.710 | −0.342 |
| RI | CGGATA | 646.60 | 451 | 0.697 | −0.360 |
| RI | CGTATC | 695.96 | 440 | 0.632 | −0.459 |
| RI | CGTATA | 253.12 | 152 | 0.601 | −0.510 |
| RI | CGGATT | 1405.93 | 825 | 0.587 | −0.533 |
| RI | CGCATA | 591.21 | 276 | 0.467 | −0.762 |
| RK | AGGAAG | 3199.71 | 4856 | 1.518 | 0.417 |
| RK | AGGAAA | 2470.61 | 3737 | 1.513 | 0.414 |
| RK | AGAAAA | 2516.58 | 3482 | 1.384 | 0.325 |
| RK | CGCAAG | 2954.85 | 2981 | 1.009 | 0.009 |
| RK | CGGAAG | 3231.73 | 3225 | 0.998 | −0.002 |
| RK | AGAAAG | 3259.25 | 2909 | 0.893 | −0.114 |
| RK | CGAAAA | 1352.67 | 1189 | 0.879 | −0.129 |
| RK | CGGAAA | 2495.33 | 1834 | 0.735 | −0.308 |
| RK | CGAAAG | 1751.85 | 1265 | 0.722 | −0.326 |
| RK | CGTAAA | 976.81 | 566 | 0.579 | −0.546 |
| RK | CGCAAA | 2281.54 | 1209 | 0.530 | −0.635 |
| RK | CGTAAG | 1265.08 | 503 | 0.398 | −0.922 |
| RL | CGCCTC | 1491.12 | 2511 | 1.684 | 0.521 |
| RL | CGCCTG | 3098.73 | 4809 | 1.552 | 0.439 |
| RL | CGGCTG | 3389.08 | 5029 | 1.484 | 0.395 |
| RL | CGGCTC | 1630.84 | 2301 | 1.411 | 0.344 |
| RL | CGTTTA | 256.76 | 337 | 1.313 | 0.272 |
| RL | AGATTA | 661.49 | 862 | 1.303 | 0.265 |
| RL | CGTCTT | 440.20 | 562 | 1.277 | 0.244 |
| RL | CGTCTA | 237.40 | 296 | 1.247 | 0.221 |
| RL | CGTTTG | 431.33 | 526 | 1.219 | 0.198 |
| RL | CGTCTC | 638.40 | 723 | 1.133 | 0.124 |
| RL | AGGCTA | 600.44 | 669 | 1.114 | 0.108 |
| RL | AGACTT | 1134.11 | 1227 | 1.082 | 0.079 |
| RL | AGGCTG | 3355.51 | 3531 | 1.052 | 0.051 |
| RL | AGACTA | 611.62 | 617 | 1.009 | 0.009 |
| RL | AGGCTT | 1113.39 | 1104 | 0.992 | −0.008 |
| RL | CGACTA | 328.75 | 324 | 0.986 | −0.015 |
| RL | CGGCTA | 606.45 | 593 | 0.978 | −0.022 |
| RL | CGTCTG | 1326.68 | 1281 | 0.966 | −0.035 |
| RL | AGGCTC | 1614.68 | 1540 | 0.954 | −0.047 |
| RL | CGATTA | 355.55 | 337 | 0.948 | −0.054 |
| RL | CGACTT | 609.59 | 576 | 0.945 | −0.057 |
| RL | CGCCTA | 554.49 | 501 | 0.904 | −0.101 |
| RL | AGGTTA | 649.40 | 586 | 0.902 | −0.103 |
| RL | CGCCTT | 1028.19 | 862 | 0.838 | −0.176 |
| RL | CGCTTG | 1007.46 | 804 | 0.798 | −0.226 |
| RL | CGGCTT | 1124.53 | 866 | 0.770 | −0.261 |
| RL | AGATTG | 1111.24 | 839 | 0.755 | −0.281 |
| RL | CGACTC | 884.04 | 663 | 0.750 | −0.288 |
| RL | AGGTTG | 1090.94 | 774 | 0.709 | −0.343 |
| RL | AGACTC | 1644.73 | 1142 | 0.694 | −0.365 |
| RL | CGATTG | 597.29 | 408 | 0.683 | −0.381 |
| RL | CGACTG | 1837.15 | 1128 | 0.614 | −0.488 |
| RL | CGCTTA | 599.71 | 345 | 0.575 | −0.553 |
| RL | CGGTTG | 1101.86 | 566 | 0.514 | −0.666 |
| RL | AGACTG | 3417.95 | 1701 | 0.498 | −0.698 |
| RL | CGGTTA | 655.90 | 297 | 0.453 | −0.792 |
| RM | CGCATG | 1558.32 | 1961 | 1.258 | 0.230 |
| RM | AGGATG | 1687.45 | 1974 | 1.170 | 0.157 |
| RM | CGAATG | 923.88 | 932 | 1.009 | 0.009 |
| RM | AGAATG | 1718.85 | 1690 | 0.983 | −0.017 |
| RM | CGGATG | 1704.33 | 1374 | 0.806 | −0.215 |
| RM | CGTATG | 667.17 | 329 | 0.493 | −0.707 |
| RN | AGAAAT | 1568.88 | 2627 | 1.674 | 0.515 |
| RN | AGGAAC | 1696.37 | 2200 | 1.297 | 0.260 |
| RN | AGGAAT | 1540.22 | 1796 | 1.166 | 0.154 |
| RN | AGAAAC | 1727.93 | 1949 | 1.128 | 0.120 |
| RN | CGAAAT | 843.28 | 930 | 1.103 | 0.098 |
| RN | CGCAAC | 1566.55 | 1575 | 1.005 | 0.005 |
| RN | CGGAAC | 1713.34 | 1621 | 0.946 | −0.055 |
| RN | CGAAAC | 928.77 | 784 | 0.844 | −0.169 |
| RN | CGGAAT | 1555.63 | 1002 | 0.644 | −0.440 |
| RN | CGTAAT | 608.96 | 340 | 0.558 | −0.583 |
| RN | CGCAAT | 1422.36 | 711 | 0.500 | −0.693 |
| RN | CGTAAC | 670.70 | 308 | 0.459 | −0.778 |
| RP | CGGCCG | 587.88 | 1226 | 2.085 | 0.735 |
| RP | CGGCCC | 1622.47 | 2939 | 1.811 | 0.594 |
| RP | CGCCCG | 537.51 | 717 | 1.334 | 0.288 |
| RP | AGGCCC | 1606.39 | 1982 | 1.234 | 0.210 |
| RP | AGGCCG | 582.05 | 666 | 1.144 | 0.135 |
| RP | AGGCCT | 1438.75 | 1642 | 1.141 | 0.132 |
| RP | AGGCCA | 1388.57 | 1511 | 1.088 | 0.084 |
| RP | CGTCCT | 568.84 | 589 | 1.035 | 0.035 |
| RP | AGACCA | 1414.41 | 1387 | 0.981 | −0.020 |
| RP | CGGCCT | 1453.14 | 1390 | 0.957 | −0.044 |
| RP | AGACCT | 1465.52 | 1398 | 0.954 | −0.047 |
| RP | CGTCCC | 635.12 | 582 | 0.916 | −0.087 |
| RP | CGGCCA | 1402.47 | 1285 | 0.916 | −0.087 |
| RP | CGCCCC | 1483.46 | 1320 | 0.890 | −0.117 |
| RP | CGTCCA | 549.00 | 487 | 0.887 | −0.120 |
| RP | AGACCC | 1636.29 | 1283 | 0.784 | −0.243 |
| RP | CGACCA | 760.25 | 591 | 0.777 | −0.252 |
| RP | CGACCC | 879.51 | 671 | 0.763 | −0.271 |
| RP | CGACCT | 787.72 | 580 | 0.736 | −0.306 |
| RP | CGCCCA | 1282.31 | 887 | 0.692 | −0.369 |
| RP | CGTCCG | 230.13 | 159 | 0.691 | −0.370 |
| RP | CGCCCT | 1328.65 | 830 | 0.625 | −0.470 |
| RP | CGACCG | 318.68 | 184 | 0.577 | −0.549 |
| RP | AGACCG | 592.88 | 246 | 0.415 | −0.880 |
| RQ | AGACAA | 1054.78 | 1456 | 1.380 | 0.322 |
| RQ | CGGCAG | 2920.52 | 3950 | 1.352 | 0.302 |
| RQ | CGCCAG | 2670.31 | 3160 | 1.183 | 0.168 |
| RQ | AGGCAA | 1035.51 | 1177 | 1.137 | 0.128 |
| RQ | AGGCAG | 2891.59 | 3013 | 1.042 | 0.041 |
| RQ | CGACAA | 566.95 | 522 | 0.921 | −0.083 |
| RQ | CGTCAG | 1143.25 | 953 | 0.834 | −0.182 |
| RQ | CGTCAA | 409.41 | 327 | 0.799 | −0.225 |
| RQ | CGACAG | 1583.16 | 1249 | 0.789 | −0.237 |
| RQ | CGGCAA | 1045.87 | 763 | 0.730 | −0.315 |
| RQ | AGACAG | 2945.39 | 2062 | 0.700 | −0.357 |
| RQ | CGCCAA | 956.27 | 591 | 0.618 | −0.481 |
| RR | CGCCGC | 1172.08 | 2232 | 1.904 | 0.644 |
| RR | CGGCGG | 1402.02 | 2316 | 1.652 | 0.502 |
| RR | AGAAGA | 1426.00 | 2307 | 1.618 | 0.481 |
| RR | CGGCGC | 1281.90 | 2064 | 1.610 | 0.476 |
| RR | AGGAGG | 1374.38 | 1973 | 1.436 | 0.362 |
| RR | CGCCGG | 1281.90 | 1679 | 1.310 | 0.270 |
| RR | CGAAGA | 766.48 | 987 | 1.288 | 0.253 |
| RR | AGGAGA | 1399.95 | 1758 | 1.256 | 0.228 |
| RR | CGCAGG | 1269.20 | 1565 | 1.233 | 0.209 |
| RR | CGGAGG | 1388.13 | 1670 | 1.203 | 0.185 |
| RR | CGTCGT | 214.84 | 228 | 1.061 | 0.059 |
| RR | CGAAGG | 752.48 | 770 | 1.023 | 0.023 |
| RR | CGCCGT | 501.81 | 502 | 1.000 | 0.000 |
| RR | AGAAGG | 1399.95 | 1325 | 0.946 | −0.055 |
| RR | CGGCGT | 548.83 | 498 | 0.907 | −0.097 |
| RR | CGTCGA | 297.51 | 265 | 0.891 | −0.116 |
| RR | CGGCGA | 760.01 | 675 | 0.888 | −0.119 |
| RR | CGTCGC | 501.81 | 438 | 0.873 | −0.136 |
| RR | AGGCGG | 1388.13 | 1177 | 0.848 | −0.165 |
| RR | CGTCGG | 548.83 | 450 | 0.820 | −0.199 |
| RR | CGACGT | 297.51 | 241 | 0.810 | −0.211 |
| RR | CGCCGA | 694.89 | 547 | 0.787 | −0.239 |
| RR | AGGCGA | 752.48 | 570 | 0.757 | −0.278 |
| RR | CGGAGA | 1413.96 | 1068 | 0.755 | −0.281 |
| RR | AGACGA | 766.48 | 557 | 0.727 | −0.319 |
| RR | AGGCGT | 543.39 | 383 | 0.705 | −0.350 |
| RR | AGGCGC | 1269.20 | 889 | 0.700 | −0.356 |
| RR | AGACGT | 553.50 | 376 | 0.679 | −0.387 |
| RR | CGACGA | 411.98 | 272 | 0.660 | −0.415 |
| RR | CGCAGA | 1292.82 | 771 | 0.596 | −0.517 |
| RR | CGACGG | 760.01 | 411 | 0.541 | −0.615 |
| RR | CGACGC | 694.89 | 368 | 0.530 | −0.636 |
| RR | CGTAGA | 553.50 | 271 | 0.490 | −0.714 |
| RR | CGTAGG | 543.39 | 235 | 0.432 | −0.838 |
| RR | AGACGC | 1292.82 | 524 | 0.405 | −0.903 |
| RR | AGACGG | 1413.96 | 569 | 0.402 | −0.910 |
| RS | CGCTCG | 332.61 | 817 | 2.456 | 0.899 |
| RS | CGCAGC | 1425.00 | 2853 | 2.002 | 0.694 |
| RS | CGCTCC | 1257.78 | 2184 | 1.736 | 0.552 |
| RS | AGAAGT | 991.66 | 1532 | 1.545 | 0.435 |
| RS | CGTTCT | 468.44 | 687 | 1.467 | 0.383 |
| RS | CGAAGT | 533.02 | 728 | 1.366 | 0.312 |
| RS | CGTTCC | 538.50 | 707 | 1.313 | 0.272 |
| RS | AGGAGC | 1543.09 | 1992 | 1.291 | 0.255 |
| RS | CGTTCA | 378.71 | 471 | 1.244 | 0.218 |
| RS | CGGAGC | 1558.53 | 1856 | 1.191 | 0.175 |
| RS | AGGAGT | 973.54 | 1071 | 1.100 | 0.095 |
| RS | AGAAGC | 1571.80 | 1628 | 1.036 | 0.035 |
| RS | AGATCA | 975.67 | 1000 | 1.025 | 0.025 |
| RS | CGAAGC | 844.85 | 859 | 1.017 | 0.017 |
| RS | CGCTCA | 884.55 | 860 | 0.972 | −0.028 |
| RS | CGCAGT | 899.04 | 853 | 0.949 | −0.053 |
| RS | AGATCT | 1206.86 | 1106 | 0.916 | −0.087 |
| RS | CGCTCT | 1094.14 | 942 | 0.861 | −0.150 |
| RS | CGTTCG | 142.40 | 121 | 0.850 | −0.163 |
| RS | AGGTCA | 957.85 | 808 | 0.844 | −0.170 |
| RS | CGATCA | 524.43 | 416 | 0.793 | −0.232 |
| RS | AGGTCT | 1184.81 | 939 | 0.793 | −0.233 |
| RS | AGGTCG | 360.17 | 284 | 0.789 | −0.238 |
| RS | CGATCT | 648.69 | 497 | 0.766 | −0.266 |
| RS | AGGTCC | 1362.00 | 1036 | 0.761 | −0.274 |
| RS | CGGAGT | 983.28 | 745 | 0.758 | −0.278 |
| RS | CGTAGT | 384.91 | 278 | 0.722 | −0.325 |
| RS | CGGTCG | 363.77 | 235 | 0.646 | −0.437 |
| RS | CGATCC | 745.70 | 455 | 0.610 | −0.494 |
| RS | AGATCC | 1387.35 | 830 | 0.598 | −0.514 |
| RS | CGGTCC | 1375.63 | 821 | 0.597 | −0.516 |
| RS | CGATCG | 197.19 | 107 | 0.543 | −0.611 |
| RS | CGGTCA | 967.43 | 507 | 0.524 | −0.646 |
| RS | CGTAGC | 610.09 | 317 | 0.520 | −0.655 |
| RS | AGATCG | 366.87 | 177 | 0.482 | −0.729 |
| RS | CGGTCT | 1196.66 | 518 | 0.433 | −0.837 |
| RT | CGCACG | 450.78 | 858 | 1.903 | 0.644 |
| RT | AGAACT | 1083.61 | 1467 | 1.354 | 0.303 |
| RT | CGCACC | 1372.27 | 1821 | 1.327 | 0.283 |
| RT | AGGACG | 488.14 | 646 | 1.323 | 0.280 |
| RT | AGGACT | 1063.81 | 1389 | 1.306 | 0.267 |
| RT | AGAACA | 1225.34 | 1575 | 1.285 | 0.251 |
| RT | AGGACA | 1202.96 | 1523 | 1.266 | 0.236 |
| RT | AGGACC | 1485.98 | 1773 | 1.193 | 0.177 |
| RT | CGGACG | 493.02 | 537 | 1.089 | 0.085 |
| RT | CGAACA | 658.62 | 661 | 1.004 | 0.004 |
| RT | CGAACT | 582.44 | 556 | 0.955 | −0.046 |
| RT | CGGACC | 1500.85 | 1408 | 0.938 | −0.064 |
| RT | CGCACA | 1110.90 | 984 | 0.886 | −0.121 |
| RT | CGGACA | 1215.00 | 949 | 0.781 | −0.247 |
| RT | AGAACC | 1513.63 | 1166 | 0.770 | −0.261 |
| RT | CGTACT | 420.60 | 313 | 0.744 | −0.295 |
| RT | CGAACC | 813.58 | 599 | 0.736 | −0.306 |
| RT | CGGACT | 1074.45 | 712 | 0.663 | −0.411 |
| RT | CGCACT | 982.40 | 638 | 0.649 | −0.432 |
| RT | CGTACC | 587.52 | 361 | 0.614 | −0.487 |
| RT | AGAACG | 497.22 | 302 | 0.607 | −0.499 |
| RT | CGTACA | 475.62 | 288 | 0.606 | −0.502 |
| RT | CGAACG | 267.26 | 154 | 0.576 | −0.551 |
| RT | CGTACG | 193.00 | 79 | 0.409 | −0.893 |
| RV | CGTGTG | 889.90 | 1699 | 1.909 | 0.647 |
| RV | CGTGTC | 449.83 | 826 | 1.836 | 0.608 |
| RV | CGAGTA | 315.92 | 562 | 1.779 | 0.576 |
| RV | CGTGTA | 228.14 | 391 | 1.714 | 0.539 |
| RV | CGTGTT | 351.34 | 565 | 1.608 | 0.475 |
| RV | AGAGTT | 905.17 | 1350 | 1.491 | 0.400 |
| RV | AGAGTA | 587.76 | 876 | 1.490 | 0.399 |
| RV | CGAGTC | 622.91 | 914 | 1.467 | 0.383 |
| RV | CGAGTT | 486.53 | 681 | 1.400 | 0.336 |
| RV | CGAGTG | 1232.31 | 1576 | 1.279 | 0.246 |
| RV | CGGGTC | 1149.12 | 1310 | 1.140 | 0.131 |
| RV | AGGGTC | 1137.73 | 1221 | 1.073 | 0.071 |
| RV | CGGGTG | 2273.30 | 2328 | 1.024 | 0.024 |
| RV | AGAGTC | 1158.91 | 1154 | 0.996 | −0.004 |
| RV | CGCGTG | 2078.54 | 1725 | 0.830 | −0.186 |
| RV | AGGGTA | 577.02 | 471 | 0.816 | −0.203 |
| RV | AGAGTG | 2292.67 | 1750 | 0.763 | −0.270 |
| RV | CGGGTA | 582.79 | 438 | 0.752 | −0.286 |
| RV | AGGGTG | 2250.78 | 1658 | 0.737 | −0.306 |
| RV | CGCGTC | 1050.67 | 763 | 0.726 | −0.320 |
| RV | AGGGTT | 888.63 | 645 | 0.726 | −0.320 |
| RV | CGGGTT | 897.52 | 548 | 0.611 | −0.493 |
| RV | CGCGTA | 532.86 | 132 | 0.248 | −1.395 |
| RV | CGCGTT | 820.63 | 178 | 0.217 | −1.528 |
| RW | CGCTGG | 1038.00 | 2199 | 2.118 | 0.751 |
| RW | CGTTGG | 444.40 | 380 | 0.855 | −0.157 |
| RW | AGGTGG | 1124.01 | 876 | 0.779 | −0.249 |
| RW | CGATGG | 615.40 | 466 | 0.757 | −0.278 |
| RW | AGATGG | 1144.93 | 804 | 0.702 | −0.353 |
| RW | CGGTGG | 1135.26 | 777 | 0.684 | −0.379 |
| RY | CGCTAC | 1173.12 | 2612 | 2.227 | 0.800 |
| RY | CGCTAT | 953.25 | 1198 | 1.257 | 0.229 |
| RY | CGTTAC | 502.25 | 565 | 1.125 | 0.118 |
| RY | CGTTAT | 408.12 | 459 | 1.125 | 0.117 |
| RY | AGATAT | 1051.45 | 1018 | 0.968 | −0.032 |
| RY | AGATAC | 1293.97 | 1239 | 0.958 | −0.043 |
| RY | CGATAT | 565.15 | 509 | 0.901 | −0.105 |
| RY | CGATAC | 695.51 | 584 | 0.840 | −0.175 |
| RY | AGGTAC | 1270.33 | 1007 | 0.793 | −0.232 |
| RY | AGGTAT | 1032.24 | 769 | 0.745 | −0.294 |
| RY | CGGTAC | 1283.04 | 856 | 0.667 | −0.405 |
| RY | CGGTAT | 1042.57 | 455 | 0.436 | −0.829 |
| SA | TCGGCG | 241.39 | 778 | 3.223 | 1.170 |
| SA | TCGGCC | 892.76 | 1976 | 2.213 | 0.795 |
| SA | TCAGCA | 1366.87 | 2526 | 1.848 | 0.614 |
| SA | TCTGCA | 1690.75 | 3035 | 1.795 | 0.585 |
| SA | TCTGCT | 1931.41 | 3350 | 1.734 | 0.551 |
| SA | TCAGCT | 1561.43 | 2630 | 1.684 | 0.521 |
| SA | AGTGCT | 1587.01 | 2487 | 1.567 | 0.449 |
| SA | AGTGCA | 1389.27 | 2040 | 1.468 | 0.384 |
| SA | AGTGCC | 2413.15 | 3437 | 1.424 | 0.354 |
| SA | TCAGCC | 2374.25 | 3294 | 1.387 | 0.327 |
| SA | TCGGCT | 587.12 | 808 | 1.376 | 0.319 |
| SA | TCTGCC | 2936.83 | 3480 | 1.185 | 0.170 |
| SA | TCGGCA | 513.97 | 598 | 1.163 | 0.151 |
| SA | TCTGCG | 794.06 | 745 | 0.938 | −0.064 |
| SA | TCAGCG | 641.95 | 584 | 0.910 | −0.095 |
| SA | AGTGCG | 652.47 | 532 | 0.815 | −0.204 |
| SA | AGCGCG | 1034.18 | 802 | 0.775 | −0.254 |
| SA | AGCGCC | 3824.90 | 2428 | 0.635 | −0.454 |
| SA | TCCGCG | 912.82 | 577 | 0.632 | −0.459 |
| SA | TCCGCC | 3376.05 | 1230 | 0.364 | −1.010 |
| SA | AGCGCT | 2515.45 | 709 | 0.282 | −1.266 |
| SA | AGCGCA | 2202.02 | 601 | 0.273 | −1.299 |
| SA | TCCGCA | 1943.61 | 476 | 0.245 | −1.407 |
| SA | TCCGCT | 2220.26 | 481 | 0.217 | −1.530 |
| SC | TCCTGC | 1640.34 | 2828 | 1.724 | 0.545 |
| SC | AGCTGC | 1858.43 | 3034 | 1.633 | 0.490 |
| SC | TCCTGT | 1381.63 | 1779 | 1.288 | 0.253 |
| SC | AGCTGT | 1565.33 | 1922 | 1.228 | 0.205 |
| SC | TCGTGC | 433.77 | 361 | 0.832 | −0.184 |
| SC | TCTTGT | 1201.89 | 941 | 0.783 | −0.245 |
| SC | AGTTGT | 987.57 | 698 | 0.707 | −0.347 |
| SC | TCGTGT | 365.36 | 225 | 0.616 | −0.485 |
| SC | TCATGT | 971.65 | 584 | 0.601 | −0.509 |
| SC | TCTTGC | 1426.94 | 758 | 0.531 | −0.633 |
| SC | TCATGC | 1153.59 | 525 | 0.455 | −0.787 |
| SC | AGTTGC | 1172.49 | 504 | 0.430 | −0.844 |
| SD | TCAGAT | 1978.63 | 3706 | 1.873 | 0.628 |
| SD | AGTGAT | 2011.05 | 3683 | 1.831 | 0.605 |
| SD | AGTGAC | 2271.71 | 4040 | 1.778 | 0.576 |
| SD | TCGGAC | 840.43 | 1438 | 1.711 | 0.537 |
| SD | TCTGAT | 2447.46 | 3578 | 1.462 | 0.380 |
| SD | TCAGAC | 2235.09 | 2906 | 1.300 | 0.262 |
| SD | TCGGAT | 744.00 | 840 | 1.129 | 0.121 |
| SD | TCTGAC | 2764.69 | 2949 | 1.067 | 0.065 |
| SD | AGCGAC | 3600.71 | 2017 | 0.560 | −0.580 |
| SD | TCCGAC | 3178.17 | 1336 | 0.420 | −0.867 |
| SD | AGCGAT | 3187.56 | 920 | 0.289 | −1.243 |
| SD | TCCGAT | 2813.50 | 660 | 0.235 | −1.450 |
| SE | TCAGAA | 2420.84 | 4815 | 1.989 | 0.688 |
| SE | AGTGAA | 2460.50 | 4686 | 1.904 | 0.644 |
| SE | TCGGAG | 1217.33 | 2184 | 1.794 | 0.584 |
| SE | TCTGAA | 2994.45 | 4621 | 1.543 | 0.434 |
| SE | TCAGAG | 3237.43 | 4683 | 1.447 | 0.369 |
| SE | AGTGAG | 3290.47 | 4410 | 1.340 | 0.293 |
| SE | TCTGAG | 4004.54 | 4891 | 1.221 | 0.200 |
| SE | TCGGAA | 910.28 | 879 | 0.966 | −0.035 |
| SE | AGCGAG | 5215.47 | 2961 | 0.568 | −0.566 |
| SE | TCCGAG | 4603.44 | 2005 | 0.436 | −0.831 |
| SE | AGCGAA | 3899.95 | 847 | 0.217 | −1.527 |
| SE | TCCGAA | 3442.29 | 715 | 0.208 | −1.572 |
| SF | TCCTTC | 2645.79 | 4407 | 1.666 | 0.510 |
| SF | AGCTTC | 2997.56 | 3942 | 1.315 | 0.274 |
| SF | TCATTT | 1625.65 | 1773 | 1.091 | 0.087 |
| SF | TCCTTT | 2311.58 | 2487 | 1.076 | 0.073 |
| SF | AGTTTT | 1652.29 | 1695 | 1.026 | 0.026 |
| SF | AGCTTT | 2618.91 | 2370 | 0.905 | −0.100 |
| SF | TCTTTT | 2010.85 | 1809 | 0.900 | −0.106 |
| SF | TCTTTC | 2301.58 | 1728 | 0.751 | −0.287 |
| SF | AGTTTC | 1891.18 | 1353 | 0.715 | −0.335 |
| SF | TCGTTT | 611.27 | 342 | 0.559 | −0.581 |
| SF | TCATTC | 1860.69 | 991 | 0.533 | −0.630 |
| SF | TCGTTC | 699.65 | 330 | 0.472 | −0.751 |
| SG | AGTGGT | 1051.00 | 2094 | 1.992 | 0.689 |
| SG | TCGGGG | 586.31 | 1117 | 1.905 | 0.645 |
| SG | TCGGGC | 814.29 | 1487 | 1.826 | 0.602 |
| SG | AGTGGA | 1623.36 | 2932 | 1.806 | 0.591 |
| SG | TCAGGA | 1597.19 | 2760 | 1.728 | 0.547 |
| SG | TCTGGA | 1975.64 | 3391 | 1.716 | 0.540 |
| SG | AGTGGG | 1584.81 | 2584 | 1.630 | 0.489 |
| SG | TCTGGG | 1928.73 | 2974 | 1.542 | 0.433 |
| SG | AGTGGC | 2201.05 | 3314 | 1.506 | 0.409 |
| SG | TCTGGT | 1279.07 | 1902 | 1.487 | 0.397 |
| SG | TCAGGG | 1559.26 | 2161 | 1.386 | 0.326 |
| SG | TCAGGT | 1034.06 | 1351 | 1.307 | 0.267 |
| SG | TCGGGA | 600.57 | 684 | 1.139 | 0.130 |
| SG | TCGGGT | 388.82 | 410 | 1.054 | 0.053 |
| SG | TCTGGC | 2678.70 | 2734 | 1.021 | 0.020 |
| SG | TCAGGC | 2165.57 | 2114 | 0.976 | −0.024 |
| SG | AGCGGC | 3488.72 | 2475 | 0.709 | −0.343 |
| SG | AGCGGG | 2511.96 | 1464 | 0.583 | −0.540 |
| SG | TCCGGG | 2217.18 | 1117 | 0.504 | −0.686 |
| SG | TCCGGC | 3079.31 | 1163 | 0.378 | −0.974 |
| SG | AGCGGT | 1665.85 | 536 | 0.322 | −1.134 |
| SG | AGCGGA | 2573.06 | 663 | 0.258 | −1.356 |
| SG | TCCGGA | 2271.11 | 560 | 0.247 | −1.400 |
| SG | TCCGGT | 1470.37 | 359 | 0.244 | −1.410 |
| SH | AGCCAC | 2202.27 | 3210 | 1.458 | 0.377 |
| SH | TCTCAT | 1226.22 | 1426 | 1.163 | 0.151 |
| SH | TCCCAC | 1943.83 | 2233 | 1.149 | 0.139 |
| SH | AGTCAT | 1007.57 | 1082 | 1.074 | 0.071 |
| SH | AGCCAT | 1597.01 | 1606 | 1.006 | 0.006 |
| SH | TCGCAC | 514.03 | 512 | 0.996 | −0.004 |
| SH | TCCCAT | 1409.60 | 1349 | 0.957 | −0.044 |
| SH | TCACAT | 991.32 | 929 | 0.937 | −0.065 |
| SH | AGTCAC | 1389.42 | 1077 | 0.775 | −0.255 |
| SH | TCACAC | 1367.03 | 956 | 0.699 | −0.358 |
| SH | TCTCAC | 1690.94 | 1158 | 0.685 | −0.379 |
| SH | TCGCAT | 372.75 | 174 | 0.467 | −0.762 |
| SI | TCCATC | 2374.96 | 4526 | 1.906 | 0.645 |
| SI | AGCATC | 2690.72 | 4471 | 1.662 | 0.508 |
| SI | TCCATT | 1878.09 | 2383 | 1.269 | 0.238 |
| SI | AGCATT | 2127.79 | 2384 | 1.120 | 0.114 |
| SI | TCCATA | 863.76 | 963 | 1.115 | 0.109 |
| SI | AGTATA | 617.40 | 640 | 1.037 | 0.036 |
| SI | TCAATA | 607.45 | 618 | 1.017 | 0.017 |
| SI | AGTATT | 1342.43 | 1299 | 0.968 | −0.033 |
| SI | AGCATA | 978.60 | 943 | 0.964 | −0.037 |
| SI | TCTATA | 751.38 | 658 | 0.876 | −0.133 |
| SI | TCTATT | 1633.75 | 1215 | 0.744 | −0.296 |
| SI | TCAATT | 1320.79 | 957 | 0.725 | −0.322 |
| SI | AGTATC | 1697.59 | 924 | 0.544 | −0.608 |
| SI | TCGATA | 228.41 | 109 | 0.477 | −0.740 |
| SI | TCTATC | 2065.98 | 958 | 0.464 | −0.769 |
| SI | TCGATT | 496.64 | 185 | 0.373 | −0.988 |
| SI | TCAATC | 1670.22 | 557 | 0.333 | −1.098 |
| SI | TCGATC | 628.03 | 184 | 0.293 | −1.228 |
| SK | TCCAAG | 3563.99 | 5021 | 1.409 | 0.343 |
| SK | TCCAAA | 2751.88 | 3634 | 1.321 | 0.278 |
| SK | AGCAAG | 4037.83 | 5128 | 1.270 | 0.239 |
| SK | AGCAAA | 3117.75 | 3736 | 1.198 | 0.181 |
| SK | TCAAAA | 1935.30 | 2282 | 1.179 | 0.165 |
| SK | AGTAAA | 1967.01 | 2149 | 1.093 | 0.088 |
| SK | TCAAAG | 2506.42 | 2082 | 0.831 | −0.186 |
| SK | TCTAAA | 2393.86 | 1838 | 0.768 | −0.264 |
| SK | TCGAAG | 942.46 | 522 | 0.554 | −0.591 |
| SK | AGTAAG | 2547.49 | 1300 | 0.510 | −0.673 |
| SK | TCTAAG | 3100.32 | 1569 | 0.506 | −0.681 |
| SK | TCGAAA | 727.71 | 331 | 0.455 | −0.788 |
| SL | AGTTTA | 709.05 | 1103 | 1.556 | 0.442 |
| SL | TCGCTG | 1355.42 | 2104 | 1.552 | 0.440 |
| SL | TCCTTG | 1666.44 | 2462 | 1.477 | 0.390 |
| SL | TCTTTA | 862.92 | 1267 | 1.468 | 0.384 |
| SL | AGCCTC | 2794.39 | 4013 | 1.436 | 0.362 |
| SL | TCTTTG | 1449.64 | 2009 | 1.386 | 0.326 |
| SL | TCATTA | 697.62 | 862 | 1.236 | 0.212 |
| SL | AGCCTG | 5807.08 | 7014 | 1.208 | 0.189 |
| SL | AGTTTG | 1191.15 | 1427 | 1.198 | 0.181 |
| SL | TCGCTC | 652.23 | 777 | 1.191 | 0.175 |
| SL | TCTCTA | 797.87 | 950 | 1.191 | 0.175 |
| SL | TCTCTT | 1479.47 | 1750 | 1.183 | 0.168 |
| SL | TCCCTG | 5125.62 | 6034 | 1.177 | 0.163 |
| SL | TCCCTC | 2466.46 | 2805 | 1.137 | 0.129 |
| SL | TCCTTA | 991.98 | 1076 | 1.085 | 0.081 |
| SL | AGTCTT | 1215.66 | 1242 | 1.022 | 0.021 |
| SL | AGCCTT | 1926.85 | 1959 | 1.017 | 0.017 |
| SL | TCACTA | 645.03 | 630 | 0.977 | −0.024 |
| SL | AGCTTG | 1888.00 | 1786 | 0.946 | −0.056 |
| SL | TCACTT | 1196.06 | 1111 | 0.929 | −0.074 |
| SL | TCCCTT | 1700.73 | 1545 | 0.908 | −0.096 |
| SL | TCCCTA | 917.19 | 810 | 0.883 | −0.124 |
| SL | AGTCTA | 655.60 | 569 | 0.868 | −0.142 |
| SL | TCATTG | 1171.95 | 1015 | 0.866 | −0.144 |
| SL | AGCCTA | 1039.14 | 875 | 0.842 | −0.172 |
| SL | TCTCTC | 2145.58 | 1760 | 0.820 | −0.198 |
| SL | TCTCTG | 4458.78 | 3418 | 0.767 | −0.266 |
| SL | AGCTTA | 1123.86 | 758 | 0.674 | −0.394 |
| SL | AGTCTC | 1763.00 | 1158 | 0.657 | −0.420 |
| SL | TCGTTG | 440.67 | 280 | 0.635 | −0.454 |
| SL | TCACTC | 1734.58 | 1100 | 0.634 | −0.455 |
| SL | TCACTG | 3604.66 | 2254 | 0.625 | −0.470 |
| SL | TCGCTT | 449.74 | 279 | 0.620 | −0.477 |
| SL | TCGCTA | 242.54 | 143 | 0.590 | −0.528 |
| SL | TCGTTA | 262.32 | 140 | 0.534 | −0.628 |
| SL | AGTCTG | 3663.72 | 1808 | 0.493 | −0.706 |
| SM | TCCATG | 2282.65 | 3908 | 1.712 | 0.538 |
| SM | AGCATG | 2586.13 | 3300 | 1.276 | 0.244 |
| SM | TCAATG | 1605.31 | 1129 | 0.703 | −0.352 |
| SM | TCGATG | 603.62 | 365 | 0.605 | −0.503 |
| SM | AGTATG | 1631.61 | 966 | 0.592 | −0.524 |
| SM | TCTATG | 1985.68 | 1027 | 0.517 | −0.659 |
| SN | AGCAAC | 2539.42 | 3717 | 1.464 | 0.381 |
| SN | TCCAAC | 2241.42 | 3216 | 1.435 | 0.361 |
| SN | TCAAAT | 1431.22 | 1883 | 1.316 | 0.274 |
| SN | AGCAAT | 2305.68 | 2513 | 1.090 | 0.086 |
| SN | TCCAAT | 2035.11 | 2000 | 0.983 | −0.017 |
| SN | AGTAAT | 1454.67 | 1425 | 0.980 | −0.021 |
| SN | AGTAAC | 1602.14 | 1339 | 0.836 | −0.179 |
| SN | TCAAAC | 1576.31 | 1194 | 0.757 | −0.278 |
| SN | TCTAAT | 1770.34 | 1297 | 0.733 | −0.311 |
| SN | TCTAAC | 1949.81 | 955 | 0.490 | −0.714 |
| SN | TCGAAT | 538.16 | 258 | 0.479 | −0.735 |
| SN | TCGAAC | 592.72 | 240 | 0.405 | −0.904 |
| SP | TCGCCG | 282.21 | 549 | 1.945 | 0.665 |
| SP | TCGCCC | 778.87 | 1221 | 1.568 | 0.450 |
| SP | TCCCCG | 1067.21 | 1621 | 1.519 | 0.418 |
| SP | TCTCCA | 2214.76 | 3119 | 1.408 | 0.342 |
| SP | AGCCCC | 3336.96 | 4654 | 1.395 | 0.333 |
| SP | TCTCCT | 2294.78 | 2888 | 1.259 | 0.230 |
| SP | AGCCCG | 1209.10 | 1432 | 1.184 | 0.169 |
| SP | TCCCCA | 2545.99 | 2968 | 1.166 | 0.153 |
| SP | TCACCA | 1790.50 | 1869 | 1.044 | 0.043 |
| SP | AGCCCT | 2988.71 | 3086 | 1.033 | 0.032 |
| SP | AGTCCT | 1885.59 | 1904 | 1.010 | 0.010 |
| SP | TCACCT | 1855.20 | 1752 | 0.944 | −0.057 |
| SP | AGCCCA | 2884.48 | 2607 | 0.904 | −0.101 |
| SP | TCCCCT | 2637.98 | 2238 | 0.848 | −0.164 |
| SP | AGTCCA | 1819.84 | 1473 | 0.809 | −0.211 |
| SP | TCGCCT | 697.59 | 562 | 0.806 | −0.216 |
| SP | TCGCCA | 673.26 | 541 | 0.804 | −0.219 |
| SP | TCTCCC | 2562.18 | 2036 | 0.795 | −0.230 |
| SP | TCACCC | 2071.37 | 1568 | 0.757 | −0.278 |
| SP | AGTCCC | 2105.31 | 1534 | 0.729 | −0.317 |
| SP | TCTCCG | 928.37 | 664 | 0.715 | −0.335 |
| SP | TCCCCC | 2945.37 | 2058 | 0.699 | −0.358 |
| SP | TCACCG | 750.53 | 426 | 0.568 | −0.566 |
| SP | AGTCCG | 762.83 | 319 | 0.418 | −0.872 |
| SQ | TCCCAG | 4427.95 | 5592 | 1.263 | 0.233 |
| SQ | AGCCAG | 5016.65 | 6041 | 1.204 | 0.186 |
| SQ | TCTCAA | 1379.40 | 1644 | 1.192 | 0.175 |
| SQ | AGTCAA | 1133.44 | 1293 | 1.141 | 0.132 |
| SQ | TCACAA | 1115.16 | 1196 | 1.072 | 0.070 |
| SQ | AGCCAA | 1796.52 | 1819 | 1.013 | 0.012 |
| SQ | TCCCAA | 1585.70 | 1474 | 0.930 | −0.073 |
| SQ | TCTCAG | 3851.88 | 3430 | 0.890 | −0.116 |
| SQ | TCGCAG | 1170.92 | 1015 | 0.867 | −0.143 |
| SQ | TCACAG | 3114.02 | 2271 | 0.729 | −0.316 |
| SQ | AGTCAG | 3165.04 | 2215 | 0.700 | −0.357 |
| SQ | TCGCAA | 419.32 | 186 | 0.444 | −0.813 |
| SR | AGCCGC | 1540.23 | 2828 | 1.836 | 0.608 |
| SR | TCCAGG | 1472.14 | 2309 | 1.568 | 0.450 |
| SR | AGCCGG | 1684.56 | 2353 | 1.397 | 0.334 |
| SR | TCCCGG | 1486.87 | 1976 | 1.329 | 0.284 |
| SR | AGCAGG | 1667.87 | 2186 | 1.311 | 0.271 |
| SR | AGCCGT | 659.43 | 857 | 1.300 | 0.262 |
| SR | TCGCGC | 359.50 | 446 | 1.241 | 0.216 |
| SR | TCCAGA | 1499.54 | 1850 | 1.234 | 0.210 |
| SR | TCAAGA | 1054.57 | 1294 | 1.227 | 0.205 |
| SR | TCGCGG | 393.19 | 481 | 1.223 | 0.202 |
| SR | TCCCGC | 1359.49 | 1605 | 1.181 | 0.166 |
| SR | TCTCGA | 701.14 | 826 | 1.178 | 0.164 |
| SR | AGTCGT | 416.04 | 484 | 1.163 | 0.151 |
| SR | TCCCGA | 806.00 | 937 | 1.163 | 0.151 |
| SR | AGCAGA | 1698.90 | 1925 | 1.133 | 0.125 |
| SR | AGCCGA | 913.16 | 1020 | 1.117 | 0.111 |
| SR | TCTCGT | 506.32 | 493 | 0.974 | −0.027 |
| SR | AGTCGA | 576.12 | 553 | 0.960 | −0.041 |
| SR | TCCCGT | 582.04 | 553 | 0.950 | −0.051 |
| SR | TCAAGG | 1035.31 | 922 | 0.891 | −0.116 |
| SR | TCGAGG | 389.29 | 324 | 0.832 | −0.184 |
| SR | TCTCGG | 1293.43 | 1062 | 0.821 | −0.197 |
| SR | TCACGT | 409.33 | 323 | 0.789 | −0.237 |
| SR | AGTAGA | 1071.85 | 746 | 0.696 | −0.362 |
| SR | TCGCGT | 153.92 | 102 | 0.663 | −0.411 |
| SR | AGTCGG | 1062.80 | 675 | 0.635 | −0.454 |
| SR | AGTCGC | 971.74 | 591 | 0.608 | −0.497 |
| SR | TCACGA | 566.83 | 344 | 0.607 | −0.499 |
| SR | TCGAGA | 396.54 | 240 | 0.605 | −0.502 |
| SR | TCTAGA | 1304.45 | 750 | 0.575 | −0.553 |
| SR | TCGCGA | 213.14 | 115 | 0.540 | −0.617 |
| SR | TCTCGC | 1182.62 | 636 | 0.538 | −0.620 |
| SR | TCACGG | 1045.66 | 534 | 0.511 | −0.672 |
| SR | TCTAGG | 1280.62 | 574 | 0.448 | −0.802 |
| SR | TCACGC | 956.08 | 406 | 0.425 | −0.856 |
| SR | AGTAGG | 1052.27 | 443 | 0.421 | −0.865 |
| SS | AGCAGC | 3919.72 | 7160 | 1.827 | 0.602 |
| SS | TCGTCG | 213.54 | 376 | 1.761 | 0.566 |
| SS | TCCTCG | 807.53 | 1302 | 1.612 | 0.478 |
| SS | TCCAGC | 3459.74 | 4832 | 1.397 | 0.334 |
| SS | TCTTCA | 1868.19 | 2596 | 1.390 | 0.329 |
| SS | AGCAGT | 2472.97 | 3417 | 1.382 | 0.323 |
| SS | TCCTCC | 3053.74 | 4162 | 1.363 | 0.310 |
| SS | TCTTCT | 2310.85 | 2896 | 1.253 | 0.226 |
| SS | TCCAGT | 2182.77 | 2691 | 1.233 | 0.209 |
| SS | TCATCA | 1510.32 | 1795 | 1.188 | 0.173 |
| SS | AGCTCC | 3459.74 | 4024 | 1.163 | 0.151 |
| SS | TCATCT | 1868.19 | 2118 | 1.134 | 0.126 |
| SS | TCCTCA | 2147.58 | 2413 | 1.124 | 0.117 |
| SS | AGCTCG | 914.89 | 1001 | 1.094 | 0.090 |
| SS | TCCTCT | 2656.45 | 2744 | 1.033 | 0.032 |
| SS | TCGTCC | 807.53 | 818 | 1.013 | 0.013 |
| SS | TCTTCC | 2656.45 | 2600 | 0.979 | −0.021 |
| SS | AGTTCT | 1898.79 | 1856 | 0.977 | −0.023 |
| SS | AGTTCA | 1535.06 | 1498 | 0.976 | −0.024 |
| SS | TCAAGT | 1535.06 | 1404 | 0.915 | −0.089 |
| SS | AGCTCA | 2433.11 | 2075 | 0.853 | −0.159 |
| SS | AGCTCT | 3009.63 | 2465 | 0.819 | −0.200 |
| SS | TCTTCG | 702.47 | 556 | 0.791 | −0.234 |
| SS | TCATCC | 2147.58 | 1632 | 0.760 | −0.275 |
| SS | AGTAGT | 1560.21 | 1030 | 0.660 | −0.415 |
| SS | AGTTCC | 2182.77 | 1405 | 0.644 | −0.441 |
| SS | TCGTCT | 702.47 | 434 | 0.618 | −0.482 |
| SS | TCATCG | 567.91 | 343 | 0.604 | −0.504 |
| SS | TCGTCA | 567.91 | 313 | 0.551 | −0.596 |
| SS | TCTAGT | 1898.79 | 957 | 0.504 | −0.685 |
| SS | TCGAGC | 914.89 | 440 | 0.481 | −0.732 |
| SS | AGTAGC | 2472.97 | 1158 | 0.468 | −0.759 |
| SS | TCAAGC | 2433.11 | 1117 | 0.459 | −0.779 |
| SS | TCGAGT | 577.21 | 259 | 0.449 | −0.801 |
| SS | AGTTCG | 577.21 | 251 | 0.435 | −0.833 |
| SS | TCTAGC | 3009.63 | 899 | 0.299 | −1.208 |
| ST | TCCACG | 785.52 | 1434 | 1.826 | 0.602 |
| ST | AGCACC | 2709.18 | 4149 | 1.531 | 0.426 |
| ST | TCCACC | 2391.25 | 3527 | 1.475 | 0.389 |
| ST | AGCACG | 889.95 | 1180 | 1.326 | 0.282 |
| ST | AGCACA | 2193.18 | 2692 | 1.227 | 0.205 |
| ST | TCCACA | 1935.81 | 2329 | 1.203 | 0.185 |
| ST | TCCACT | 1711.89 | 1937 | 1.131 | 0.124 |
| ST | AGCACT | 1939.49 | 2193 | 1.131 | 0.123 |
| ST | TCAACA | 1361.39 | 1485 | 1.091 | 0.087 |
| ST | TCAACT | 1203.91 | 1270 | 1.055 | 0.053 |
| ST | TCTACT | 1489.18 | 1390 | 0.933 | −0.069 |
| ST | TCTACA | 1683.97 | 1461 | 0.868 | −0.142 |
| ST | AGTACT | 1223.64 | 1036 | 0.847 | −0.166 |
| ST | AGTACA | 1383.69 | 1061 | 0.767 | −0.266 |
| ST | TCGACG | 207.72 | 145 | 0.698 | −0.359 |
| ST | TCTACC | 2080.15 | 1218 | 0.586 | −0.535 |
| ST | TCGACC | 632.34 | 365 | 0.577 | −0.550 |
| ST | AGTACC | 1709.24 | 976 | 0.571 | −0.560 |
| ST | TCGACT | 452.69 | 240 | 0.530 | −0.635 |
| ST | TCAACC | 1681.68 | 873 | 0.519 | −0.656 |
| ST | TCAACG | 552.43 | 275 | 0.498 | −0.698 |
| ST | TCGACA | 511.90 | 236 | 0.461 | −0.774 |
| ST | TCTACG | 683.32 | 302 | 0.442 | −0.817 |
| ST | AGTACG | 561.48 | 201 | 0.358 | −1.027 |
| SV | TCGGTG | 935.47 | 1822 | 1.948 | 0.667 |
| SV | TCTGTA | 788.92 | 1398 | 1.772 | 0.572 |
| SV | TCTGTT | 1214.96 | 2136 | 1.758 | 0.564 |
| SV | TCAGTA | 637.79 | 1121 | 1.758 | 0.564 |
| SV | AGTGTT | 998.32 | 1719 | 1.722 | 0.543 |
| SV | TCAGTT | 982.23 | 1591 | 1.620 | 0.482 |
| SV | TCTGTC | 1555.54 | 2367 | 1.522 | 0.420 |
| SV | AGTGTC | 1278.17 | 1943 | 1.520 | 0.419 |
| SV | TCTGTG | 3077.33 | 4672 | 1.518 | 0.418 |
| SV | AGTGTA | 648.24 | 976 | 1.506 | 0.409 |
| SV | TCGGTC | 472.87 | 683 | 1.444 | 0.368 |
| SV | TCAGTG | 2487.84 | 2925 | 1.176 | 0.162 |
| SV | AGTGTG | 2528.60 | 2901 | 1.147 | 0.137 |
| SV | TCAGTC | 1257.56 | 1351 | 1.074 | 0.072 |
| SV | TCGGTA | 239.82 | 231 | 0.963 | −0.037 |
| SV | TCGGTT | 369.33 | 266 | 0.720 | −0.328 |
| SV | AGCGTC | 2025.93 | 1298 | 0.641 | −0.445 |
| SV | TCCGTG | 3537.57 | 2065 | 0.584 | −0.538 |
| SV | AGCGTG | 4007.89 | 2221 | 0.554 | −0.590 |
| SV | TCCGTC | 1788.18 | 829 | 0.464 | −0.769 |
| SV | AGCGTT | 1582.36 | 446 | 0.282 | −1.266 |
| SV | TCCGTA | 906.91 | 239 | 0.264 | −1.334 |
| SV | TCCGTT | 1396.67 | 329 | 0.236 | −1.446 |
| SV | AGCGTA | 1027.48 | 217 | 0.211 | −1.555 |
| SW | TCCTGG | 1756.97 | 2825 | 1.608 | 0.475 |
| SW | AGCTGG | 1990.56 | 2404 | 1.208 | 0.189 |
| SW | TCGTGG | 464.61 | 444 | 0.956 | −0.045 |
| SW | TCTTGG | 1528.39 | 1137 | 0.744 | −0.296 |
| SW | TCATGG | 1235.61 | 778 | 0.630 | −0.463 |
| SW | AGTTGG | 1255.86 | 644 | 0.513 | −0.668 |
| SY | TCCTAC | 1871.53 | 3038 | 1.623 | 0.484 |
| SY | AGCTAC | 2120.35 | 2864 | 1.351 | 0.301 |
| SY | TCCTAT | 1520.75 | 1869 | 1.229 | 0.206 |
| SY | AGCTAT | 1722.94 | 1609 | 0.934 | −0.068 |
| SY | AGTTAT | 1087.01 | 1010 | 0.929 | −0.073 |
| SY | AGTTAC | 1337.74 | 1153 | 0.862 | −0.149 |
| SY | TCATAT | 1069.49 | 897 | 0.839 | −0.176 |
| SY | TCTTAT | 1322.91 | 1100 | 0.832 | −0.185 |
| SY | TCTTAC | 1628.04 | 1204 | 0.740 | −0.302 |
| SY | TCGTAC | 494.91 | 304 | 0.614 | −0.487 |
| SY | TCGTAT | 402.15 | 204 | 0.507 | −0.679 |
| SY | TCATAC | 1316.18 | 642 | 0.488 | −0.718 |
| TA | ACGGCG | 348.71 | 734 | 2.105 | 0.744 |
| TA | ACAGCA | 1829.79 | 3283 | 1.794 | 0.585 |
| TA | ACGGCC | 1289.71 | 2090 | 1.621 | 0.483 |
| TA | ACTGCA | 1618.13 | 2557 | 1.580 | 0.458 |
| TA | ACAGCT | 2090.24 | 3295 | 1.576 | 0.455 |
| TA | ACTGCT | 1848.45 | 2764 | 1.495 | 0.402 |
| TA | ACAGCC | 3178.34 | 3912 | 1.231 | 0.208 |
| TA | ACGGCA | 742.49 | 804 | 1.083 | 0.080 |
| TA | ACTGCC | 2810.69 | 3015 | 1.073 | 0.070 |
| TA | ACGGCT | 848.18 | 804 | 0.948 | −0.053 |
| TA | ACAGCG | 859.36 | 803 | 0.934 | −0.068 |
| TA | ACTGCG | 759.96 | 623 | 0.820 | −0.199 |
| TA | ACCGCG | 1061.55 | 584 | 0.550 | −0.598 |
| TA | ACCGCC | 3926.11 | 1648 | 0.420 | −0.868 |
| TA | ACCGCA | 2260.29 | 561 | 0.248 | −1.394 |
| TA | ACCGCT | 2582.01 | 577 | 0.223 | −1.498 |
| TC | ACCTGC | 1892.82 | 3247 | 1.715 | 0.540 |
| TC | ACCTGT | 1594.30 | 1994 | 1.251 | 0.224 |
| TC | ACGTGC | 621.78 | 691 | 1.111 | 0.106 |
| TC | ACGTGT | 523.72 | 484 | 0.924 | −0.079 |
| TC | ACTTGT | 1141.35 | 1033 | 0.905 | −0.100 |
| TC | ACATGT | 1290.64 | 938 | 0.727 | −0.319 |
| TC | ACTTGC | 1355.07 | 815 | 0.601 | −0.508 |
| TC | ACATGC | 1532.31 | 750 | 0.489 | −0.714 |
| TD | ACAGAT | 2415.25 | 4195 | 1.737 | 0.552 |
| TD | ACAGAC | 2728.31 | 3765 | 1.380 | 0.322 |
| TD | ACTGAT | 2135.87 | 2913 | 1.364 | 0.310 |
| TD | ACGGAC | 1107.10 | 1446 | 1.306 | 0.267 |
| TD | ACTGAC | 2412.71 | 2615 | 1.084 | 0.081 |
| TD | ACGGAT | 980.07 | 922 | 0.941 | −0.061 |
| TD | ACCGAC | 3370.20 | 1547 | 0.459 | −0.779 |
| TD | ACCGAT | 2983.49 | 730 | 0.245 | −1.408 |
| TE | ACAGAA | 3127.33 | 5307 | 1.697 | 0.529 |
| TE | ACGGAG | 1697.07 | 2517 | 1.483 | 0.394 |
| TE | ACTGAA | 2765.58 | 4093 | 1.480 | 0.392 |
| TE | ACAGAG | 4182.23 | 5419 | 1.296 | 0.259 |
| TE | ACTGAG | 3698.46 | 4124 | 1.115 | 0.109 |
| TE | ACGGAA | 1269.01 | 1080 | 0.851 | −0.161 |
| TE | ACCGAG | 5166.20 | 2450 | 0.474 | −0.746 |
| TE | ACCGAA | 3863.10 | 779 | 0.202 | −1.601 |
| TF | ACCTTC | 3026.54 | 4955 | 1.637 | 0.493 |
| TF | ACATTT | 2140.61 | 2275 | 1.063 | 0.061 |
| TF | ACTTTT | 1893.00 | 1904 | 1.006 | 0.006 |
| TF | ACCTTT | 2644.23 | 2518 | 0.952 | −0.049 |
| TF | ACTTTC | 2166.69 | 1822 | 0.841 | −0.173 |
| TF | ACGTTT | 868.62 | 650 | 0.748 | −0.290 |
| TF | ACGTTC | 994.21 | 666 | 0.670 | −0.401 |
| TF | ACATTC | 2450.10 | 1394 | 0.569 | −0.564 |
| TG | ACTGGA | 1710.74 | 3660 | 2.139 | 0.761 |
| TG | ACTGGT | 1107.57 | 1887 | 1.704 | 0.533 |
| TG | ACAGGA | 1934.51 | 2970 | 1.535 | 0.429 |
| TG | ACGGGC | 1064.34 | 1583 | 1.487 | 0.397 |
| TG | ACTGGG | 1670.12 | 2322 | 1.390 | 0.330 |
| TG | ACGGGG | 766.35 | 1049 | 1.369 | 0.314 |
| TG | ACAGGT | 1252.44 | 1694 | 1.353 | 0.302 |
| TG | ACAGGG | 1888.57 | 2148 | 1.137 | 0.129 |
| TG | ACTGGC | 2319.53 | 2620 | 1.130 | 0.122 |
| TG | ACAGGC | 2622.93 | 2664 | 1.016 | 0.016 |
| TG | ACGGGT | 508.22 | 484 | 0.952 | −0.049 |
| TG | ACGGGA | 784.99 | 710 | 0.904 | −0.100 |
| TG | ACCGGG | 2332.90 | 1093 | 0.469 | −0.758 |
| TG | ACCGGC | 3240.03 | 1373 | 0.424 | −0.859 |
| TG | ACCGGT | 1547.11 | 355 | 0.229 | −1.472 |
| TG | ACCGGA | 2389.65 | 528 | 0.221 | −1.510 |
| TH | ACTCAT | 1054.95 | 1291 | 1.224 | 0.202 |
| TH | ACCCAC | 2032.09 | 2408 | 1.185 | 0.170 |
| TH | ACGCAC | 667.53 | 764 | 1.145 | 0.135 |
| TH | ACACAT | 1192.94 | 1186 | 0.994 | −0.006 |
| TH | ACTCAC | 1454.76 | 1384 | 0.951 | −0.050 |
| TH | ACCCTT | 1473.60 | 1287 | 0.873 | −0.135 |
| TH | ACACAC | 1645.05 | 1383 | 0.841 | −0.174 |
| TH | ACGCAT | 484.07 | 302 | 0.624 | −0.472 |
| TI | ACCATC | 2842.70 | 5915 | 2.081 | 0.733 |
| TI | ACCATT | 2247.97 | 2878 | 1.280 | 0.247 |
| TI | ACAATA | 836.96 | 980 | 1.171 | 0.158 |
| TI | ACCATA | 1033.87 | 1137 | 1.100 | 0.095 |
| TI | ACAATT | 1819.82 | 1579 | 0.868 | −0.142 |
| TI | ACTATA | 740.14 | 642 | 0.867 | −0.142 |
| TI | ACTATT | 1609.31 | 1337 | 0.831 | −0.185 |
| TI | ACGATA | 339.62 | 190 | 0.559 | −0.581 |
| TI | ACGATT | 738.45 | 389 | 0.527 | −0.641 |
| TI | ACGATC | 933.81 | 463 | 0.496 | −0.702 |
| TI | ACTATC | 2035.08 | 942 | 0.463 | −0.770 |
| TI | ACAATC | 2301.27 | 1027 | 0.446 | −0.807 |
| TK | ACCAAG | 3878.56 | 6678 | 1.722 | 0.543 |
| TK | ACCAAA | 2994.77 | 3789 | 1.265 | 0.235 |
| TK | ACAAAA | 2424.38 | 2546 | 1.050 | 0.049 |
| TK | ACAAAG | 3139.84 | 2507 | 0.798 | −0.225 |
| TK | ACTAAA | 2143.95 | 1684 | 0.785 | −0.241 |
| TK | ACGAAG | 1274.09 | 708 | 0.556 | −0.588 |
| TK | ACGAAA | 983.77 | 511 | 0.519 | −0.655 |
| TK | ACTAAG | 2776.65 | 1193 | 0.430 | −0.845 |
| TL | ACGCTG | 1815.48 | 3357 | 1.849 | 0.615 |
| TL | ACTTTA | 765.72 | 1207 | 1.576 | 0.455 |
| TL | ACTTTG | 1286.34 | 1876 | 1.458 | 0.377 |
| TL | ACATTA | 865.87 | 1115 | 1.288 | 0.253 |
| TL | ACCTTG | 1796.82 | 2257 | 1.256 | 0.228 |
| TL | ACTCTA | 707.99 | 876 | 1.237 | 0.213 |
| TL | ACGCTC | 873.61 | 1057 | 1.210 | 0.191 |
| TL | ACCCTC | 2659.44 | 3133 | 1.178 | 0.164 |
| TL | ACCCTG | 5526.65 | 6354 | 1.150 | 0.140 |
| TL | ACTCTT | 1312.81 | 1469 | 1.119 | 0.112 |
| TL | ACACTA | 800.60 | 799 | 0.998 | −0.002 |
| TL | ACGCTA | 324.87 | 307 | 0.945 | −0.057 |
| TL | ACCTTA | 1069.59 | 957 | 0.895 | −0.111 |
| TL | ACACTT | 1484.53 | 1316 | 0.886 | −0.121 |
| TL | ACGTTG | 590.25 | 505 | 0.856 | −0.156 |
| TL | ACATTG | 1454.60 | 1210 | 0.832 | −0.184 |
| TL | ACCCTT | 1833.80 | 1515 | 0.826 | −0.191 |
| TL | ACCCTA | 988.95 | 802 | 0.811 | −0.210 |
| TL | ACTCTG | 3956.51 | 3120 | 0.789 | −0.238 |
| TL | ACGTTA | 351.36 | 262 | 0.746 | −0.293 |
| TL | ACTCTC | 1903.88 | 1391 | 0.731 | −0.314 |
| TL | ACGCTT | 602.39 | 427 | 0.709 | −0.344 |
| TL | ACACTG | 4474.03 | 3013 | 0.673 | −0.395 |
| TL | ACACTC | 2152.92 | 1274 | 0.592 | −0.525 |
| TM | ACCATG | 2733.42 | 4467 | 1.634 | 0.491 |
| TM | ACAATG | 2212.81 | 1641 | 0.742 | −0.299 |
| TM | ACGATG | 897.92 | 655 | 0.729 | −0.315 |
| TM | ACTATG | 1956.85 | 1038 | 0.530 | −0.634 |
| TN | ACCAAC | 2378.62 | 4300 | 1.808 | 0.592 |
| TN | ACAAAT | 1748.34 | 2194 | 1.255 | 0.227 |
| TN | ACCAAT | 2159.68 | 2454 | 1.136 | 0.128 |
| TN | ACAAAC | 1925.59 | 1486 | 0.772 | −0.259 |
| TN | ACTAAT | 1546.11 | 1077 | 0.697 | −0.362 |
| TN | ACGAAT | 709.45 | 336 | 0.474 | −0.747 |
| TN | ACTAAC | 1702.85 | 789 | 0.463 | −0.769 |
| TN | ACGAAC | 781.37 | 316 | 0.404 | −0.905 |
| TP | ACGCCG | 349.03 | 632 | 1.811 | 0.594 |
| TP | ACGCCC | 963.29 | 1491 | 1.548 | 0.437 |
| TP | ACTCCA | 1814.66 | 2359 | 1.300 | 0.262 |
| TP | ACCCCG | 1062.52 | 1331 | 1.253 | 0.225 |
| TP | ACTCCT | 1880.23 | 2186 | 1.163 | 0.151 |
| TP | ACACCA | 2052.02 | 2361 | 1.151 | 0.140 |
| TP | ACCCCA | 2534.80 | 2784 | 1.098 | 0.094 |
| TP | ACACCT | 2126.17 | 2104 | 0.990 | −0.010 |
| TP | ACCCCT | 2626.39 | 2415 | 0.920 | −0.084 |
| TP | ACGCCA | 832.67 | 748 | 0.898 | −0.107 |
| TP | ACCCCC | 2932.43 | 2380 | 0.812 | −0.209 |
| TP | ACACCC | 2373.91 | 1922 | 0.810 | −0.211 |
| TP | ACGCCT | 862.76 | 697 | 0.808 | −0.213 |
| TP | ACTCCC | 2099.31 | 1649 | 0.785 | −0.241 |
| TP | ACTCCG | 760.66 | 538 | 0.707 | −0.346 |
| TP | ACACCG | 860.15 | 534 | 0.621 | −0.477 |
| TQ | ACTCAA | 1103.35 | 1368 | 1.240 | 0.215 |
| TQ | ACCCAG | 4303.71 | 5173 | 1.202 | 0.184 |
| TQ | ACGCAG | 1413.75 | 1518 | 1.074 | 0.071 |
| TQ | ACACAA | 1247.67 | 1328 | 1.064 | 0.062 |
| TQ | ACTCAG | 3081.01 | 2839 | 0.921 | −0.082 |
| TQ | ACCCAA | 1541.21 | 1410 | 0.915 | −0.089 |
| TQ | ACACAG | 3484.02 | 2765 | 0.794 | −0.231 |
| TQ | ACGCAA | 506.28 | 280 | 0.553 | −0.592 |
| TR | ACCAGG | 1331.08 | 2049 | 1.539 | 0.431 |
| TR | ACGCGC | 403.79 | 605 | 1.498 | 0.404 |
| TR | ACGCGG | 441.63 | 661 | 1.497 | 0.403 |
| TR | ACTCGA | 521.72 | 717 | 1.374 | 0.318 |
| TR | ACAAGA | 1097.61 | 1429 | 1.302 | 0.264 |
| TR | ACCCGC | 1229.22 | 1547 | 1.259 | 0.230 |
| TR | ACCCGG | 1344.40 | 1668 | 1.241 | 0.216 |
| TR | ACTCGT | 376.76 | 448 | 1.189 | 0.173 |
| TR | ACCAGA | 1355.85 | 1599 | 1.179 | 0.165 |
| TR | ACCCGA | 728.77 | 758 | 1.040 | 0.039 |
| TR | ACCCGT | 526.27 | 535 | 1.017 | 0.016 |
| TR | ACAAGG | 1077.56 | 1072 | 0.995 | −0.005 |
| TR | ACGAGG | 437.25 | 433 | 0.990 | −0.010 |
| TR | ACTCGG | 962.45 | 823 | 0.855 | −0.157 |
| TR | ACGCGT | 172.88 | 141 | 0.816 | −0.204 |
| TR | ACACGT | 426.04 | 329 | 0.772 | −0.258 |
| TR | ACGAGA | 445.39 | 331 | 0.743 | −0.297 |
| TR | ACACGA | 589.97 | 432 | 0.732 | −0.312 |
| TR | ACACGG | 1088.34 | 756 | 0.695 | −0.364 |
| TR | ACTCGC | 879.99 | 607 | 0.690 | −0.371 |
| TR | ACTAGA | 970.65 | 624 | 0.643 | −0.442 |
| TR | ACGCGA | 239.40 | 150 | 0.627 | −0.468 |
| TR | ACACGC | 995.10 | 498 | 0.500 | −0.692 |
| TR | ACTAGG | 952.91 | 383 | 0.402 | −0.911 |
| TS | ACCAGC | 2807.29 | 4575 | 1.630 | 0.488 |
| TS | ACCTCG | 655.24 | 1060 | 1.618 | 0.481 |
| TS | ACGTCG | 215.24 | 348 | 1.617 | 0.480 |
| TS | ACTTCA | 1247.51 | 1844 | 1.478 | 0.391 |
| TS | ACTTCT | 1543.11 | 1974 | 1.279 | 0.246 |
| TS | ACATCA | 1410.69 | 1754 | 1.243 | 0.218 |
| TS | ACCAGT | 1771.14 | 2194 | 1.239 | 0.214 |
| TS | ACCTCC | 2477.85 | 3050 | 1.231 | 0.208 |
| TS | ACCTCA | 1742.59 | 1938 | 1.112 | 0.106 |
| TS | ACATCT | 1744.95 | 1911 | 1.095 | 0.091 |
| TS | ACGTCC | 813.96 | 840 | 1.032 | 0.031 |
| TS | ACCTCT | 2155.49 | 2072 | 0.961 | −0.040 |
| TS | ACAAGT | 1433.80 | 1335 | 0.931 | −0.071 |
| TS | ACTTCC | 1773.89 | 1524 | 0.859 | −0.152 |
| TS | ACGTCA | 572.43 | 450 | 0.786 | −0.241 |
| TS | ACATCC | 2005.92 | 1570 | 0.783 | −0.245 |
| TS | ACTTCG | 469.09 | 353 | 0.753 | −0.284 |
| TS | ACGTCT | 708.07 | 527 | 0.744 | −0.295 |
| TS | ACATCG | 530.44 | 361 | 0.681 | −0.385 |
| TS | ACTAGT | 1267.95 | 725 | 0.572 | −0.559 |
| TS | ACAAGC | 2272.61 | 1275 | 0.561 | −0.578 |
| TS | ACGAGT | 581.81 | 297 | 0.510 | −0.672 |
| TS | ACGAGC | 922.18 | 469 | 0.509 | −0.676 |
| TS | ACTAGC | 2009.73 | 687 | 0.342 | −1.073 |
| TT | ACCACG | 875.88 | 1567 | 1.789 | 0.582 |
| TT | ACCACC | 2666.32 | 4767 | 1.788 | 0.581 |
| TT | ACCACA | 2158.49 | 2882 | 1.335 | 0.289 |
| TT | ACCACT | 1908.81 | 2309 | 1.210 | 0.190 |
| TT | ACAACA | 1747.38 | 1793 | 1.026 | 0.026 |
| TT | ACAACT | 1545.26 | 1567 | 1.014 | 0.014 |
| TT | ACGACG | 287.72 | 252 | 0.876 | −0.133 |
| TT | ACTACT | 1366.51 | 1065 | 0.779 | −0.249 |
| TT | ACTACA | 1545.26 | 1196 | 0.774 | −0.256 |
| TT | ACGACC | 875.88 | 575 | 0.656 | −0.421 |
| TT | ACGACA | 709.06 | 437 | 0.616 | −0.484 |
| TT | ACAACC | 2158.49 | 1310 | 0.607 | −0.499 |
| TT | ACGACT | 627.04 | 357 | 0.569 | −0.563 |
| TT | ACTACC | 1908.81 | 992 | 0.520 | −0.655 |
| TT | ACAACG | 709.06 | 365 | 0.515 | −0.664 |
| TT | ACTACG | 627.04 | 283 | 0.451 | −0.796 |
| TV | ACTGTA | 845.20 | 1425 | 1.686 | 0.522 |
| TV | ACTGTT | 1301.64 | 2058 | 1.581 | 0.458 |
| TV | ACGGTG | 1512.80 | 2306 | 1.524 | 0.422 |
| TV | ACAGTA | 955.76 | 1371 | 1.434 | 0.361 |
| TV | ACTGTC | 1666.51 | 2289 | 1.374 | 0.317 |
| TV | ACAGTT | 1471.90 | 2019 | 1.372 | 0.316 |
| TV | ACTGTG | 3296.87 | 4505 | 1.366 | 0.312 |
| TV | ACGGTC | 764.70 | 911 | 1.191 | 0.175 |
| TV | ACAGTG | 3728.11 | 4108 | 1.102 | 0.097 |
| TV | ACAGTC | 1884.50 | 1933 | 1.026 | 0.025 |
| TV | ACGGTA | 387.83 | 286 | 0.737 | −0.305 |
| TV | ACGGTT | 597.27 | 415 | 0.695 | −0.364 |
| TV | ACCGTG | 4605.23 | 2640 | 0.573 | −0.556 |
| TV | ACCGTC | 2327.87 | 1285 | 0.552 | −0.594 |
| TV | ACCGTT | 1818.19 | 496 | 0.273 | −1.299 |
| TV | ACCGTA | 1180.62 | 298 | 0.252 | −1.377 |
| TW | ACGTGG | 606.25 | 837 | 1.381 | 0.323 |
| TW | ACCTGG | 1845.52 | 2403 | 1.302 | 0.264 |
| TW | ACATGG | 1494.02 | 1089 | 0.729 | −0.316 |
| TW | ACTTGG | 1321.21 | 938 | 0.710 | −0.343 |
| TY | ACCTAC | 2130.11 | 3648 | 1.713 | 0.538 |
| TY | ACCTAT | 1730.88 | 1778 | 1.027 | 0.027 |
| TY | ACTTAC | 1524.94 | 1383 | 0.907 | −0.098 |
| TY | ACGTAC | 699.73 | 621 | 0.887 | −0.119 |
| TY | ACATAT | 1401.21 | 1136 | 0.811 | −0.210 |
| TY | ACTTAT | 1239.13 | 907 | 0.732 | −0.312 |
| TY | ACGTAT | 568.59 | 408 | 0.718 | −0.332 |
| TY | ACATAC | 1724.41 | 1138 | 0.660 | −0.416 |
| VA | GTGGCC | 6082.92 | 9316 | 1.532 | 0.426 |
| VA | GTAGCA | 897.78 | 1347 | 1.500 | 0.406 |
| VA | GTTGCT | 1579.41 | 2217 | 1.404 | 0.339 |
| VA | GTAGCT | 1025.57 | 1407 | 1.372 | 0.316 |
| VA | GTGGCT | 4000.44 | 5252 | 1.313 | 0.272 |
| VA | GTGGCG | 1644.71 | 2099 | 1.276 | 0.244 |
| VA | GTTGCA | 1382.62 | 1728 | 1.250 | 0.223 |
| VA | GTGGCA | 3501.98 | 3859 | 1.102 | 0.097 |
| VA | GTAGCC | 1559.44 | 1363 | 0.874 | −0.135 |
| VA | GTTGCC | 2401.60 | 1808 | 0.753 | −0.284 |
| VA | GTAGCG | 421.64 | 216 | 0.512 | −0.669 |
| VA | GTTGCG | 649.35 | 234 | 0.360 | −1.021 |
| VA | GTCGCG | 831.37 | 284 | 0.342 | −1.074 |
| VA | GTCGCC | 3074.82 | 992 | 0.323 | −1.131 |
| VA | GTCGCT | 2022.16 | 406 | 0.201 | −1.606 |
| VA | GTCGCA | 1770.19 | 318 | 0.180 | −1.717 |
| VC | GTCTGC | 1410.66 | 2160 | 1.531 | 0.426 |
| VC | GTCTGT | 1188.18 | 1572 | 1.323 | 0.280 |
| VC | GTTTGT | 928.03 | 942 | 1.015 | 0.015 |
| VC | GTATGT | 602.60 | 594 | 0.986 | −0.014 |
| VC | GTGTGC | 2790.71 | 2583 | 0.926 | −0.077 |
| VC | GTGTGT | 2350.57 | 1996 | 0.849 | −0.164 |
| VC | GTTTGC | 1101.80 | 830 | 0.753 | −0.283 |
| VC | GTATGC | 715.44 | 411 | 0.574 | −0.554 |
| VD | GTAGAT | 1225.65 | 1924 | 1.570 | 0.451 |
| VD | GTGGAC | 5400.58 | 7734 | 1.432 | 0.359 |
| VD | GTTGAT | 1887.55 | 2389 | 1.266 | 0.236 |
| VD | GTGGAT | 4780.91 | 5727 | 1.198 | 0.181 |
| VD | GTAGAC | 1384.52 | 1346 | 0.972 | −0.028 |
| VD | GTTGAC | 2132.21 | 1791 | 0.840 | −0.174 |
| VD | GTCGAC | 2729.91 | 602 | 0.221 | −1.512 |
| VD | GTCGAT | 2416.67 | 445 | 0.184 | −1.692 |
| VE | GTAGAA | 1456.83 | 2855 | 1.960 | 0.673 |
| VE | GTGGAG | 7599.48 | 11579 | 1.524 | 0.421 |
| VE | GTTGAA | 2243.56 | 2905 | 1.295 | 0.258 |
| VE | GTGGAA | 5682.64 | 6229 | 1.096 | 0.092 |
| VE | GTAGAG | 1948.24 | 2002 | 1.028 | 0.027 |
| VE | GTTGAG | 3000.36 | 1987 | 0.662 | −0.412 |
| VE | GTCGAG | 3841.42 | 721 | 0.188 | −1.673 |
| VE | GTCGAA | 2872.48 | 367 | 0.128 | −2.058 |
| VF | GTCTTC | 2309.08 | 4216 | 1.826 | 0.602 |
| VF | GTATTT | 1023.16 | 1512 | 1.478 | 0.391 |
| VF | GTCTTT | 2017.40 | 2238 | 1.109 | 0.104 |
| VF | GTTTTT | 1575.70 | 1706 | 1.083 | 0.079 |
| VF | GTTTTC | 1803.52 | 1604 | 0.889 | −0.117 |
| VF | GTGTTT | 3991.02 | 3257 | 0.816 | −0.203 |
| VF | GTGTTC | 4568.05 | 3205 | 0.702 | −0.354 |
| VF | GTATTC | 1171.09 | 721 | 0.616 | −0.485 |
| VG | GTTGGT | 779.74 | 1617 | 2.074 | 0.729 |
| VG | GTTGGA | 1204.37 | 2315 | 1.922 | 0.653 |
| VG | GTGGGC | 4136.07 | 5977 | 1.445 | 0.368 |
| VG | GTAGGA | 782.04 | 1089 | 1.393 | 0.331 |
| VG | GTTGGG | 1175.77 | 1510 | 1.284 | 0.250 |
| VG | GTTGGC | 1632.96 | 1794 | 1.099 | 0.094 |
| VG | GTAGGT | 506.31 | 554 | 1.094 | 0.090 |
| VG | GTGGGG | 2978.07 | 3255 | 1.093 | 0.089 |
| VG | GTGGGT | 1974.96 | 2009 | 1.017 | 0.017 |
| VG | GTAGGG | 763.47 | 683 | 0.895 | −0.111 |
| VG | GTGGGA | 3050.51 | 2599 | 0.852 | −0.160 |
| VG | GTAGGC | 1060.34 | 676 | 0.638 | −0.450 |
| VG | GTCGGG | 1505.36 | 734 | 0.488 | −0.718 |
| VG | GTCGGC | 2090.72 | 734 | 0.351 | −1.047 |
| VG | GTCGGT | 998.31 | 292 | 0.292 | −1.229 |
| VG | GTCGGA | 1541.98 | 343 | 0.222 | −1.503 |
| VH | GTTCAT | 911.79 | 1418 | 1.555 | 0.442 |
| VH | GTACAT | 592.06 | 773 | 1.306 | 0.267 |
| VH | GTCCAC | 1609.82 | 2085 | 1.295 | 0.259 |
| VH | GTCCAT | 1167.39 | 1313 | 1.125 | 0.118 |
| VH | GTTCAC | 1257.35 | 1319 | 1.049 | 0.048 |
| VH | GTGCAC | 3184.70 | 2856 | 0.897 | −0.109 |
| VH | GTACAC | 816.44 | 613 | 0.751 | −0.287 |
| VH | GTGCAT | 2309.44 | 1472 | 0.637 | −0.450 |
| VI | GTCATC | 2367.78 | 5207 | 2.199 | 0.788 |
| VI | GTCATT | 1872.41 | 2827 | 1.510 | 0.412 |
| VI | GTAATA | 436.74 | 614 | 1.406 | 0.341 |
| VI | GTAATT | 949.63 | 1074 | 1.131 | 0.123 |
| VI | GTTATT | 1462.46 | 1595 | 1.091 | 0.087 |
| VI | GTCATA | 861.15 | 904 | 1.050 | 0.049 |
| VI | GTTATA | 672.60 | 702 | 1.044 | 0.043 |
| VI | GTGATT | 3704.20 | 2742 | 0.740 | −0.301 |
| VI | GTGATC | 4684.19 | 3353 | 0.716 | −0.334 |
| VI | GTGATA | 1703.61 | 1117 | 0.656 | −0.422 |
| VI | GTTATC | 1849.37 | 1053 | 0.569 | −0.563 |
| VI | GTAATC | 1200.86 | 577 | 0.480 | −0.733 |
| VK | GTAAAA | 1288.46 | 1945 | 1.510 | 0.412 |
| VK | GTCAAG | 3290.24 | 3982 | 1.210 | 0.191 |
| VK | GTGAAG | 6509.08 | 7513 | 1.154 | 0.143 |
| VK | GTAAAG | 1668.70 | 1704 | 1.021 | 0.021 |
| VK | GTCAAA | 2540.51 | 2376 | 0.935 | −0.067 |
| VK | GTTAAA | 1984.27 | 1777 | 0.896 | −0.110 |
| VK | GTGAAA | 5025.89 | 4409 | 0.877 | −0.131 |
| VK | GTTAAG | 2569.85 | 1171 | 0.456 | −0.786 |
| VL | GTTTTA | 668.83 | 1311 | 1.960 | 0.673 |
| VL | GTTCTT | 1146.70 | 1859 | 1.621 | 0.483 |
| VL | GTTTTG | 1123.58 | 1737 | 1.546 | 0.436 |
| VL | GTATTA | 434.30 | 646 | 1.487 | 0.397 |
| VL | GTCCTC | 2129.16 | 3019 | 1.418 | 0.349 |
| VL | GTTCTA | 618.41 | 832 | 1.345 | 0.297 |
| VL | GTCCTG | 4424.65 | 5574 | 1.260 | 0.231 |
| VL | GTCCTT | 1468.14 | 1722 | 1.173 | 0.159 |
| VL | GTGCTG | 8753.31 | 10107 | 1.155 | 0.144 |
| VL | GTCTTG | 1438.54 | 1628 | 1.132 | 0.124 |
| VL | GTACTA | 401.55 | 447 | 1.113 | 0.107 |
| VL | GTCCTA | 791.76 | 874 | 1.104 | 0.099 |
| VL | GTCTTA | 856.32 | 863 | 1.008 | 0.008 |
| VL | GTATTG | 729.58 | 711 | 0.975 | −0.026 |
| VL | GTACTT | 744.59 | 693 | 0.931 | −0.072 |
| VL | GTTCTC | 1662.99 | 1501 | 0.903 | −0.102 |
| VL | GTGCTC | 4212.12 | 3765 | 0.894 | −0.112 |
| VL | GTGCTA | 1566.34 | 1286 | 0.821 | −0.197 |
| VL | GTTCTG | 3455.90 | 2350 | 0.680 | −0.386 |
| VL | GTGTTG | 2845.87 | 1910 | 0.671 | −0.399 |
| VL | GTGCTT | 2904.43 | 1933 | 0.666 | −0.407 |
| VL | GTGTTA | 1694.06 | 965 | 0.570 | −0.563 |
| VL | GTACTC | 1079.84 | 541 | 0.501 | −0.691 |
| VL | GTACTG | 2244.04 | 1121 | 0.500 | −0.694 |
| VM | GTCATG | 2149.52 | 3308 | 1.539 | 0.431 |
| VM | GTGATG | 4252.41 | 3872 | 0.911 | −0.094 |
| VM | GTAATG | 1090.17 | 935 | 0.858 | −0.154 |
| VM | GTTATG | 1678.90 | 1056 | 0.629 | −0.464 |
| VN | GTCAAC | 2052.00 | 3311 | 1.614 | 0.478 |
| VN | GTAAAT | 944.92 | 1518 | 1.606 | 0.474 |
| VN | GTCAAT | 1863.13 | 2155 | 1.157 | 0.146 |
| VN | GTTAAT | 1455.20 | 1325 | 0.911 | −0.094 |
| VN | GTGAAC | 4059.49 | 3551 | 0.875 | −0.134 |
| VN | GTGAAT | 3685.83 | 3110 | 0.844 | −0.170 |
| VN | GTAAAC | 1040.71 | 854 | 0.821 | −0.198 |
| VN | GTTAAC | 1602.73 | 880 | 0.549 | −0.600 |
| VP | GTTCCT | 1434.04 | 2257 | 1.574 | 0.454 |
| VP | GTTCCA | 1384.03 | 1911 | 1.381 | 0.323 |
| VP | GTGCCC | 4055.45 | 4998 | 1.232 | 0.209 |
| VP | GTACCT | 931.17 | 1048 | 1.125 | 0.118 |
| VP | GTCCCC | 2049.96 | 2260 | 1.102 | 0.098 |
| VP | GTCCCT | 1836.02 | 2014 | 1.097 | 0.093 |
| VP | GTACCA | 898.70 | 963 | 1.072 | 0.069 |
| VP | GTCCCG | 742.77 | 786 | 1.058 | 0.057 |
| VP | GTTCCC | 1601.13 | 1506 | 0.941 | −0.061 |
| VP | GTCCCA | 1772.00 | 1596 | 0.901 | −0.105 |
| VP | GTGCCT | 3632.21 | 3062 | 0.843 | −0.171 |
| VP | GTGCCG | 1469.43 | 1228 | 0.836 | −0.179 |
| VP | GTACCC | 1039.67 | 809 | 0.778 | −0.251 |
| VP | GTGCCA | 3505.55 | 2431 | 0.693 | −0.366 |
| VP | GTTCCG | 580.15 | 279 | 0.481 | −0.732 |
| VP | GTACCG | 376.71 | 161 | 0.427 | −0.850 |
| VQ | GTACAA | 633.37 | 1049 | 1.656 | 0.505 |
| VQ | GTTCAA | 975.42 | 1485 | 1.522 | 0.420 |
| VQ | GTCCAG | 3487.32 | 3907 | 1.120 | 0.114 |
| VQ | GTACAG | 1768.65 | 1752 | 0.991 | −0.009 |
| VQ | GTTCAG | 2723.79 | 2689 | 0.987 | −0.013 |
| VQ | GTGCAG | 6898.98 | 6734 | 0.976 | −0.024 |
| VQ | GTCCAA | 1248.85 | 1067 | 0.854 | −0.157 |
| VQ | GTGCAA | 2470.60 | 1524 | 0.617 | −0.483 |
| VR | GTTCGA | 463.33 | 867 | 1.871 | 0.627 |
| VR | GTTCGT | 334.59 | 580 | 1.733 | 0.550 |
| VR | GTCCGA | 593.21 | 805 | 1.357 | 0.305 |
| VR | GTCCGC | 1000.57 | 1332 | 1.331 | 0.286 |
| VR | GTGCGC | 1979.43 | 2543 | 1.285 | 0.251 |
| VR | GTCCGT | 428.38 | 549 | 1.282 | 0.248 |
| VR | GTCCGG | 1094.32 | 1346 | 1.230 | 0.207 |
| VR | GTACGA | 300.86 | 361 | 1.200 | 0.182 |
| VR | GTAAGA | 559.73 | 660 | 1.179 | 0.165 |
| VR | GTGCGG | 2164.91 | 2552 | 1.179 | 0.164 |
| VR | GTCAGA | 1103.65 | 1291 | 1.170 | 0.157 |
| VR | GTACGT | 217.26 | 253 | 1.165 | 0.152 |
| VR | GTCAGG | 1083.48 | 1238 | 1.143 | 0.133 |
| VR | GTGAGG | 2143.46 | 1986 | 0.927 | −0.076 |
| VR | GTGCGT | 847.46 | 761 | 0.898 | −0.108 |
| VR | GTAAGG | 549.51 | 444 | 0.808 | −0.213 |
| VR | GTTCGG | 854.73 | 650 | 0.760 | −0.274 |
| VR | GTGCGA | 1173.55 | 826 | 0.704 | −0.351 |
| VR | GTTCGC | 781.50 | 545 | 0.697 | −0.360 |
| VR | GTGAGA | 2183.35 | 1511 | 0.692 | −0.368 |
| VR | GTACGG | 555.00 | 377 | 0.679 | −0.387 |
| VR | GTTAGA | 862.01 | 556 | 0.645 | −0.438 |
| VR | GTACGC | 507.46 | 286 | 0.564 | −0.573 |
| VR | GTTAGG | 846.26 | 309 | 0.365 | −1.007 |
| VS | GTTTCT | 1206.81 | 2161 | 1.791 | 0.583 |
| VS | GTCTCC | 1776.18 | 2936 | 1.653 | 0.503 |
| VS | GTCAGC | 2012.32 | 3223 | 1.602 | 0.471 |
| VS | GTTTCA | 975.63 | 1465 | 1.502 | 0.407 |
| VS | GTCAGT | 1269.59 | 1841 | 1.450 | 0.372 |
| VS | GTATCT | 783.62 | 1093 | 1.395 | 0.333 |
| VS | GTATCA | 633.51 | 806 | 1.272 | 0.241 |
| VS | GTCTCT | 1545.10 | 1847 | 1.195 | 0.178 |
| VS | GTTTCC | 1387.29 | 1604 | 1.156 | 0.145 |
| VS | GTCTCG | 469.69 | 542 | 1.154 | 0.143 |
| VS | GTCTCA | 1249.12 | 1333 | 1.067 | 0.065 |
| VS | GTGTCC | 3513.81 | 3722 | 1.059 | 0.058 |
| VS | GTGTCG | 929.19 | 860 | 0.926 | −0.077 |
| VS | GTGTCT | 3056.67 | 2784 | 0.911 | −0.093 |
| VS | GTATCC | 900.82 | 763 | 0.847 | −0.166 |
| VS | GTAAGT | 643.89 | 499 | 0.775 | −0.255 |
| VS | GTGAGC | 3980.98 | 2901 | 0.729 | −0.316 |
| VS | GTGTCA | 2471.14 | 1710 | 0.692 | −0.368 |
| VS | GTTAGT | 991.62 | 640 | 0.645 | −0.438 |
| VS | GTATCG | 238.21 | 138 | 0.579 | −0.546 |
| VS | GTTTCG | 366.85 | 202 | 0.551 | −0.597 |
| VS | GTGAGT | 2511.63 | 1371 | 0.546 | −0.605 |
| VS | GTAAGC | 1020.58 | 514 | 0.504 | −0.686 |
| VS | GTTAGC | 1571.73 | 551 | 0.351 | −1.048 |
| VT | GTCACC | 2294.69 | 4477 | 1.951 | 0.668 |
| VT | GTCACT | 1642.76 | 2452 | 1.493 | 0.401 |
| VT | GTCACG | 753.80 | 997 | 1.323 | 0.280 |
| VT | GTAACT | 833.15 | 1046 | 1.255 | 0.228 |
| VT | GTCACA | 1857.64 | 2207 | 1.188 | 0.172 |
| VT | GTAACA | 942.13 | 1096 | 1.163 | 0.151 |
| VT | GTTACT | 1283.09 | 1208 | 0.941 | −0.060 |
| VT | GTGACC | 4539.59 | 4223 | 0.930 | −0.072 |
| VT | GTGACG | 1491.24 | 1318 | 0.884 | −0.123 |
| VT | GTGACT | 3249.88 | 2758 | 0.849 | −0.164 |
| VT | GTGACA | 3674.98 | 2947 | 0.802 | −0.221 |
| VT | GTTACA | 1450.92 | 1111 | 0.766 | −0.267 |
| VT | GTAACC | 1163.79 | 758 | 0.651 | −0.429 |
| VT | GTTACC | 1792.28 | 969 | 0.541 | −0.615 |
| VT | GTAACG | 382.30 | 191 | 0.500 | −0.694 |
| VT | GTTACG | 588.76 | 183 | 0.311 | −1.169 |
| VV | GTTGTA | 655.54 | 1109 | 1.692 | 0.526 |
| VV | GTTGTT | 1009.55 | 1701 | 1.685 | 0.522 |
| VV | GTAGTA | 425.66 | 698 | 1.640 | 0.495 |
| VV | GTGGTG | 6476.64 | 9025 | 1.393 | 0.332 |
| VV | GTGGTC | 3273.84 | 4256 | 1.300 | 0.262 |
| VV | GTAGTT | 655.54 | 800 | 1.220 | 0.199 |
| VV | GTTGTC | 1292.55 | 1561 | 1.208 | 0.189 |
| VV | GTGGTA | 1660.38 | 1777 | 1.070 | 0.068 |
| VV | GTGGTT | 2557.05 | 2613 | 1.022 | 0.022 |
| VV | GTTGTG | 2557.05 | 2261 | 0.884 | −0.123 |
| VV | GTAGTG | 1660.38 | 1161 | 0.699 | −0.358 |
| VV | GTAGTC | 839.30 | 553 | 0.659 | −0.417 |
| VV | GTCGTC | 1654.87 | 858 | 0.518 | −0.657 |
| VV | GTCGTG | 3273.84 | 1250 | 0.382 | −0.963 |
| VV | GTCGTA | 839.30 | 213 | 0.254 | −1.371 |
| VV | GTCGTT | 1292.55 | 288 | 0.223 | −1.501 |
| VW | GTCTGG | 1316.29 | 1763 | 1.339 | 0.292 |
| VW | GTGTGG | 2604.03 | 2451 | 0.941 | −0.061 |
| VW | GTATGG | 667.58 | 578 | 0.866 | −0.144 |
| VW | GTTTGG | 1028.10 | 824 | 0.801 | −0.221 |
| VY | GTCTAC | 1602.79 | 2490 | 1.554 | 0.441 |
| VY | GTTTAT | 1017.23 | 1438 | 1.414 | 0.346 |
| VY | GTATAT | 660.53 | 875 | 1.325 | 0.281 |
| VY | GTCTAT | 1302.39 | 1544 | 1.186 | 0.170 |
| VY | GTGTAC | 3170.80 | 2654 | 0.837 | −0.178 |
| VY | GTTTAC | 1251.87 | 1008 | 0.805 | −0.217 |
| VY | GTATAC | 812.88 | 582 | 0.716 | −0.334 |
| VY | GTGTAT | 2576.51 | 1804 | 0.700 | −0.356 |
| WA | TGGGCA | 1469.77 | 1535 | 1.044 | 0.043 |
| WA | TGGGCG | 690.28 | 695 | 1.007 | 0.007 |
| WA | TGGGCT | 1678.97 | 1664 | 0.991 | −0.009 |
| WA | TGGGCC | 2552.98 | 2498 | 0.978 | −0.022 |
| WC | TGGTGC | 1057.38 | 1066 | 1.008 | 0.008 |
| WC | TGGTGT | 890.62 | 882 | 0.990 | −0.010 |
| WD | TGGGAC | 2699.37 | 2807 | 1.040 | 0.039 |
| WD | TGGGAT | 2389.63 | 2282 | 0.955 | −0.046 |
| WE | TGGGAG | 3580.00 | 3650 | 1.020 | 0.019 |
| WE | TGGGAA | 2677.00 | 2607 | 0.974 | −0.026 |
| WF | TGGTTT | 1639.95 | 1735 | 1.058 | 0.056 |
| WF | TGGTTC | 1877.05 | 1782 | 0.949 | −0.052 |
| WG | TGGGGT | 955.95 | 1064 | 1.113 | 0.107 |
| WG | TGGGGC | 2002.00 | 2179 | 1.088 | 0.085 |
| WG | TGGGGA | 1476.56 | 1454 | 0.985 | −0.015 |
| WG | TGGGGG | 1441.49 | 1179 | 0.818 | −0.201 |
| WH | TGGCAT | 971.42 | 1000 | 1.029 | 0.029 |
| WH | TGGCAC | 1339.58 | 1311 | 0.979 | −0.022 |
| WI | TGGATT | 1537.91 | 1627 | 1.058 | 0.056 |
| WI | TGGATA | 707.30 | 714 | 1.009 | 0.009 |
| WI | TGGATC | 1944.78 | 1849 | 0.951 | −0.051 |
| WK | TGGAAG | 3491.83 | 3645 | 1.044 | 0.043 |
| WK | TGGAAA | 2696.17 | 2543 | 0.943 | −0.058 |
| WL | TGGCTA | 683.88 | 798 | 1.167 | 0.154 |
| WL | TGGCTG | 3821.78 | 4228 | 1.106 | 0.101 |
| WL | TGGCTT | 1268.11 | 1334 | 1.052 | 0.051 |
| WL | TGGCTC | 1839.05 | 1879 | 1.022 | 0.021 |
| WL | TGGTTG | 1242.54 | 855 | 0.688 | −0.374 |
| WL | TGGTTA | 739.64 | 501 | 0.677 | −0.390 |
| WM | TGGCTG | 2335.00 | 2335 | 1.000 | 0.000 |
| WN | TGGAAT | 1978.70 | 2005 | 1.013 | 0.013 |
| WN | TGGAAC | 2179.30 | 2153 | 0.988 | −0.012 |
| WP | TGGCCC | 1302.21 | 1381 | 1.061 | 0.059 |
| WP | TGGCCG | 471.84 | 486 | 1.030 | 0.030 |
| WP | TGGCCA | 1125.64 | 1123 | 0.998 | −0.002 |
| WP | TGGCCT | 1166.31 | 1076 | 0.923 | −0.081 |
| WQ | TGGCAG | 2983.56 | 2997 | 1.005 | 0.004 |
| WQ | TGGCAA | 1068.44 | 1055 | 0.987 | −0.013 |
| WR | TGGAGG | 1198.99 | 1665 | 1.389 | 0.328 |
| WR | TGGAGA | 1221.30 | 1472 | 1.205 | 0.187 |
| WR | TGGCGG | 1210.98 | 979 | 0.808 | −0.213 |
| WR | TGGCGC | 1107.23 | 895 | 0.808 | −0.213 |
| WR | TGGCGT | 474.05 | 377 | 0.795 | −0.229 |
| WR | TGGCGA | 656.45 | 481 | 0.733 | −0.311 |
| WS | TGGAGT | 1031.75 | 1239 | 1.201 | 0.183 |
| WS | TGGAGC | 1635.35 | 1956 | 1.196 | 0.179 |
| WS | TGGTCA | 1015.12 | 898 | 0.885 | −0.123 |
| WS | TGGTCC | 1443.44 | 1271 | 0.881 | −0.127 |
| WS | TGGTCT | 1255.65 | 1076 | 0.857 | −0.154 |
| WS | TGGTCG | 381.70 | 323 | 0.846 | −0.167 |
| WT | TGGACG | 598.07 | 674 | 1.127 | 0.120 |
| WT | TGGACA | 1473.88 | 1559 | 1.058 | 0.056 |
| WT | TGGACT | 1303.39 | 1240 | 0.951 | −0.050 |
| WT | TGGACC | 1820.65 | 1723 | 0.946 | −0.055 |
| WV | TGGGTC | 1318.64 | 1378 | 1.045 | 0.044 |
| WV | TGGGTG | 2608.66 | 2633 | 1.009 | 0.009 |
| WV | TGGGTA | 668.77 | 665 | 0.994 | −0.006 |
| WV | TGGGTT | 1029.93 | 950 | 0.922 | −0.081 |
| WW | TGGTGG | 1559.00 | 1559 | 1.000 | 0.000 |
| WY | TGGTAC | 1444.91 | 1520 | 1.052 | 0.051 |
| WY | TGGTAT | 1174.09 | 1099 | 0.936 | −0.066 |
| YA | TATGCA | 1120.39 | 2249 | 2.007 | 0.697 |
| YA | TATGCT | 1279.86 | 2296 | 1.794 | 0.584 |
| YA | TATGCC | 1946.11 | 2862 | 1.471 | 0.386 |
| YA | TACGCG | 647.56 | 622 | 0.961 | −0.040 |
| YA | TATGCG | 526.19 | 482 | 0.916 | −0.088 |
| YA | TACGCC | 2395.00 | 1402 | 0.585 | −0.535 |
| YA | TACGCA | 1378.81 | 512 | 0.371 | −0.991 |
| YA | TACGCT | 1575.07 | 444 | 0.282 | −1.266 |
| YC | TACTGC | 1588.07 | 2411 | 1.518 | 0.418 |
| YC | TACTGT | 1337.61 | 1587 | 1.186 | 0.171 |
| YC | TATTGT | 1086.90 | 659 | 0.606 | −0.500 |
| YC | TATTGC | 1290.42 | 646 | 0.501 | −0.692 |
| YD | TATGAT | 2091.17 | 3707 | 1.773 | 0.572 |
| YD | TATGAC | 2362.22 | 3731 | 1.579 | 0.457 |
| YD | TACGAC | 2907.08 | 1653 | 0.569 | −0.565 |
| YD | TACGAT | 2573.52 | 843 | 0.328 | −1.116 |
| YE | TATGAA | 2515.85 | 5225 | 2.077 | 0.731 |
| YE | TATGAG | 3364.48 | 4722 | 1.403 | 0.339 |
| YE | TACGAG | 4140.53 | 2309 | 0.558 | −0.584 |
| YE | TACGAA | 3096.14 | 861 | 0.278 | −1.280 |
| YF | TACTTC | 2766.63 | 3380 | 1.222 | 0.200 |
| YF | TATTTT | 1964.12 | 2124 | 1.081 | 0.078 |
| YF | TACTTT | 2417.16 | 2201 | 0.911 | −0.094 |
| YF | TATTTC | 2248.09 | 1691 | 0.752 | −0.285 |
| YG | TATGGA | 1472.35 | 2874 | 1.952 | 0.669 |
| YG | TATGGT | 953.23 | 1665 | 1.747 | 0.558 |
| YG | TATGGG | 1437.38 | 2129 | 1.481 | 0.393 |
| YG | TATGGC | 1996.30 | 2749 | 1.377 | 0.320 |
| YG | TACGGG | 1768.93 | 1088 | 0.615 | −0.486 |
| YG | TACGGC | 2456.76 | 1484 | 0.604 | −0.504 |
| YG | TACGGT | 1173.10 | 448 | 0.382 | −0.963 |
| YG | TACGGA | 1811.96 | 633 | 0.349 | −1.052 |
| YH | TACCAC | 1862.81 | 2378 | 1.277 | 0.244 |
| YH | TACCAT | 1350.85 | 1420 | 1.051 | 0.050 |
| YH | TATCAT | 1097.67 | 1021 | 0.930 | −0.072 |
| YH | TATCAC | 1513.67 | 1006 | 0.665 | −0.409 |
| YI | TACATC | 2684.66 | 3935 | 1.466 | 0.382 |
| YI | TACATT | 2122.99 | 2162 | 1.018 | 0.018 |
| YI | TATATT | 1725.09 | 1554 | 0.901 | −0.104 |
| YI | TACATA | 976.39 | 846 | 0.866 | −0.143 |
| YI | TATATA | 793.39 | 648 | 0.817 | −0.202 |
| YI | TATATC | 2181.48 | 1339 | 0.614 | −0.488 |
| YK | TACGAG | 3508.58 | 4372 | 1.246 | 0.220 |
| YK | TACGAA | 2709.10 | 2847 | 1.051 | 0.050 |
| YK | TATGAA | 2201.34 | 2262 | 1.028 | 0.027 |
| YK | TATAAG | 2850.98 | 1789 | 0.628 | −0.466 |
| YL | TACCTG | 4522.42 | 6324 | 1.398 | 0.335 |
| YL | TATTTA | 711.20 | 966 | 1.358 | 0.306 |
| YL | TACCTC | 2176.20 | 2598 | 1.194 | 0.177 |
| YL | TACTTG | 1470.33 | 1701 | 1.157 | 0.146 |
| YL | TATTTG | 1194.75 | 1358 | 1.137 | 0.128 |
| YL | TACCTA | 809.25 | 876 | 1.082 | 0.079 |
| YL | TACCTT | 1500.58 | 1449 | 0.966 | −0.035 |
| YL | TATCTT | 1219.33 | 1166 | 0.956 | −0.045 |
| YL | TACTTA | 875.24 | 763 | 0.872 | −0.137 |
| YL | TATCTA | 657.58 | 541 | 0.823 | −0.195 |
| YL | TATCTC | 1768.32 | 1087 | 0.615 | −0.487 |
| YL | TATCTG | 3674.80 | 1751 | 0.476 | −0.741 |
| YM | TACATG | 2325.97 | 3055 | 1.313 | 0.273 |
| YM | TATATG | 1890.03 | 1161 | 0.614 | −0.487 |
| YN | TACAGC | 2442.24 | 3341 | 1.368 | 0.313 |
| YN | TACAAT | 2217.44 | 2200 | 0.992 | −0.008 |
| YN | TATAAT | 1801.83 | 1629 | 0.904 | −0.101 |
| YN | TATAAC | 1984.50 | 1276 | 0.643 | −0.442 |
| YP | TACCCG | 668.65 | 1004 | 1.502 | 0.406 |
| YP | TACCCA | 1595.15 | 1925 | 1.207 | 0.188 |
| YP | TATCCA | 1296.18 | 1438 | 1.109 | 0.104 |
| YP | TACCCC | 1845.38 | 1961 | 1.063 | 0.061 |
| YP | TATCCT | 1343.02 | 1379 | 1.027 | 0.026 |
| YP | TACCCT | 1652.79 | 1558 | 0.943 | −0.059 |
| YP | TATCCC | 1499.51 | 937 | 0.625 | −0.470 |
| YP | TATCCG | 543.32 | 242 | 0.445 | −0.809 |
| YQ | TACCAG | 3987.12 | 5013 | 1.257 | 0.229 |
| YQ | TATCTA | 1160.22 | 1179 | 1.016 | 0.016 |
| YQ | TACCAA | 1427.83 | 1397 | 0.978 | −0.022 |
| YQ | TATCAG | 3239.83 | 2226 | 0.687 | −0.375 |
| YR | TACCGC | 1307.70 | 2153 | 1.646 | 0.499 |
| YR | TACCGA | 775.30 | 990 | 1.277 | 0.244 |
| YR | TACAGA | 1442.41 | 1834 | 1.271 | 0.240 |
| YR | TACCGG | 1430.23 | 1796 | 1.256 | 0.228 |
| YR | TACAGG | 1416.06 | 1671 | 1.180 | 0.166 |
| YR | TACCGT | 559.87 | 642 | 1.147 | 0.137 |
| YR | TATCGA | 629.99 | 570 | 0.905 | −0.100 |
| YR | TATCGT | 454.94 | 383 | 0.842 | −0.172 |
| YR | TATAGA | 1172.07 | 827 | 0.706 | −0.349 |
| YR | TATCGG | 1162.17 | 629 | 0.541 | −0.614 |
| YR | TATAGG | 1150.66 | 560 | 0.487 | −0.720 |
| YR | TATCGC | 1062.60 | 509 | 0.479 | −0.736 |
| YS | TACAGC | 2204.13 | 3590 | 1.629 | 0.488 |
| YS | TACTCG | 514.46 | 783 | 1.522 | 0.420 |
| YS | TACAGT | 1390.60 | 1887 | 1.357 | 0.305 |
| YS | TATTCA | 1111.75 | 1210 | 1.088 | 0.085 |
| YS | TACTCC | 1945.47 | 2088 | 1.073 | 0.071 |
| YS | TATTCT | 1375.18 | 1466 | 1.066 | 0.064 |
| YS | TACTCA | 1368.18 | 1188 | 0.868 | −0.141 |
| YS | TATTCC | 1580.84 | 1306 | 0.826 | −0.191 |
| YS | TACTCT | 1692.37 | 1173 | 0.693 | −0.367 |
| YS | TATAGT | 1129.96 | 728 | 0.644 | −0.440 |
| YS | TATTCG | 418.04 | 229 | 0.548 | −0.602 |
| YS | TATAGC | 1791.02 | 874 | 0.488 | −0.717 |
| YT | TACACG | 697.26 | 1311 | 1.880 | 0.631 |
| YT | TACACC | 2122.58 | 2696 | 1.270 | 0.239 |
| YT | TACACA | 1718.31 | 2158 | 1.256 | 0.228 |
| YT | TACACT | 1519.54 | 1409 | 0.927 | −0.076 |
| YT | TATACT | 1234.74 | 1049 | 0.850 | −0.163 |
| YT | TATACA | 1396.25 | 1049 | 0.751 | −0.286 |
| YT | TATACC | 1724.75 | 1063 | 0.616 | −0.484 |
| YT | TATACG | 566.57 | 245 | 0.432 | −0.838 |
| YV | TATGTT | 986.79 | 1723 | 1.746 | 0.557 |
| YV | TATGTA | 640.76 | 1113 | 1.737 | 0.552 |
| YV | TATGTC | 1263.40 | 1862 | 1.474 | 0.388 |
| YV | TATGTG | 2499.39 | 3382 | 1.353 | 0.302 |
| YV | TACGTG | 3075.90 | 2279 | 0.741 | −0.300 |
| YV | TACGTC | 1554.82 | 991 | 0.637 | −0.450 |
| YV | TACGTA | 788.55 | 284 | 0.360 | −1.021 |
| YV | TACGTT | 1214.40 | 390 | 0.321 | −1.136 |
| YW | TACTGG | 1609.87 | 2212 | 1.374 | 0.318 |
| YW | TATTGG | 1308.13 | 706 | 0.540 | −0.617 |
| YY | TACTAC | 2256.03 | 2854 | 1.265 | 0.235 |
| YY | TATTAT | 1489.60 | 1459 | 0.979 | −0.021 |
| YY | TACTAT | 1833.19 | 1760 | 0.960 | −0.041 |
| YY | TATTAC | 1833.19 | 1339 | 0.730 | −0.314 |

We claim:

1. An attenuated poliovirus genome comprising:
a single active cis acting replication element (cre), said cre element located in the spacer region of the 5′-NTR between the cloverleaf and internal ribosome entry site (IRES), and
a poliovirus protein encoding sequence having a codon pair bias less than the codon pair bias of the parent poliovirus protein encoding sequence from which it is derived.

2. The attenuated poliovirus genome of claim 1, which comprises a single active cre element inserted at nucleotide 102/103.

3. The attenuated poliovirus genome of any one of claims 1-2, wherein the cre element is positioned as in SEQ ID NO:1.

4. The attenuated poliovirus genome of any one of claims 1-3, wherein the protein encoding sequence has a codon pair bias at least about 0.05 less, preferably at least about 0.1 less, more preferably at least about 0.2 less, than the codon pair bias of the parent protein encoding sequence.

5. The attenuated poliovirus genome of any one of claims 1-4, wherein the protein encoding sequence has a codon pair bias of about −0.05 or less, preferably about −0.1 or less, more preferably about −0.2 or less, more preferably about −0.3 or less, more preferably about −0.4 or less.

6. The attenuated poliovirus genome of any one of claims 1-5, wherein the protein encoding sequence is less than 90% identical, preferably less than 80% identical, to the protein encoding sequence of the parent virus.

7. The attenuated poliovirus genome of any one of claims 1-6, wherein the protein encoding sequence and the parent protein encoding sequence encode the same protein.

8. The attenuated poliovirus genome of any one of claims 1-7, wherein the parent protein encoding sequence encodes a natural isolate of the protein.

9. The attenuated poliovirus genome of any one of claims 1-8, wherein the parent protein encoding sequence is that of strain Mahoney.

10. The attenuated poliovirus genome of any one of claims 1-9, wherein the protein encoding sequence encodes a protein that differs from a natural isolate by about 10 amino acids or fewer, preferably by about 20 amino acids or fewer.

11. The attenuated poliovirus genome of any one of claims 1-10, wherein the modified protein encoding sequence comprises the sequence of PV-Min (SEQ ID NO:2) from nucleotide 755 to nucleotide 1514.

12. The attenuated poliovirus genome of any one of claims 1-11, which comprises SEQ ID NO:3.

13. An attenuated poliovirus comprising the poliovirus genome of any one of claims 1-12.

14. A method of making an attenuated poliovirus genome, which comprises:
preparing a nucleic acid sequence which comprises
a cis acting replication element (cre) located in the spacer region of the 5′-NTR between the cloverleaf and internal ribosome entry site (IRES), and
a poliovirus protein encoding sequence having a codon pair bias less than the codon pair bias of the parent poliovirus protein encoding sequence from which it is derived.

15. The method of claim 14, wherein the poliovirus protein encoding sequence of the attenuated poliovirus genome is made by rearranging the codons of the parent poliovirus nucleotide sequence to obtain a mutated nucleotide sequence that encodes the same amino acid sequence as the parent poliovirus nucleotide sequence.

16. The method of any one of claim 14 or 15, which further comprises introducing the poliovirus in to an appropriate host cell and culturing the host cell to produce the poliovirus.


SEQUENCE LISTING (3 sequences)

SEQ ID NO: 1 - DNA, 7570 bp, Artificial Sequence
  PV-1(mono-cre) attenuated  poliovirus
ttaaaacagctctggggttgtacccaccccagaggcccacgtggcggctagtactccggtattgcggtacccttgtacgcctgttttatactcccttcccgctagcactattaacaactacatacagttcaagagcaaacaccgtattgaaccagtatgtttgctagtagctagcttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatgggtgctcaggtttcatcacagaaagtgggcgcacatgaaaactcaaatagagcgtatggtagttctaccattaattacaccaccattaattattatagagattcagctagtaacgcggcttcgaaacaggacttctctcaagacccttccaagttcaccgagcccatcaaggatgtcctgataaaaacagccccaatgctaaactcgccaaacatagaggcttgcgggtatagcgatagagtactgcaattaacactgggaaactccactataaccacacaggaggcggctaattcagtagtcgcttatgggcgttggcctgaatatctgagggacagcgaagccaatccagtggaccagccgacagaaccagacgtcgctgcatgcaggttttatacgctagacaccgtgtcttggacgaaagagtcgcgagggtggtggtggaagttgcctgatgcactgagggacatgggactctttgggcaaaatatgtactaccactacctaggtaggtccgggtacaccgtgcatgtacagtgtaacgcctccaaattccaccagggggcactaggggtattcgccgtaccagagatgtgtctggccggggatagcaacaccactaccatgcacaccagctatcaaaatgccaatcctggcgagaaaggaggcactttcacgggtacgttcactcctgacaacaaccagacatcacctgcccgcaggttctgcccggtggattacctccttggaaatggcacgttgttggggaatgcctttgtgttcccgcaccagataataaacctacggaccaacaactgtgctacactggtactcccttacgtgaactccctctcgatagatagtatggtaaagcacaataattggggaattgcaatattaccattggccccattaaattttgctagtgagtcctccccagagattccaatcaccttgaccatagcccctatgtgctgtgagttcaatggattaagaaacatcaccctgccacgcttacagggcctgccggtcatgaacacccctggtagcaatcaatatcttactgcagacaacttccagtcaccgtgtgcgctgcctgaatttgatgtgaccccacctattgacatacccggtgaagtaaagaacatgatggaattggcagaaatcgacaccatgattccctttgacttaagtgccacaaaaaagaacaccatggaaatgtatagggttcggttaagtgacaaaccacatacagacgatcccatactctgcctgtcactctctccagcctcagatcctaggttgtcacatactatgcttggagaaatcctaaattactacacacactgggcaggatccctgaagttcacgtttctgttctgtggatccatgatggcaactggcaaactgttggtgtcatacgcgcctcctggagccgacccaccaaagaagcgtaaggaggcgatgttgggaacacatgtgatctgggacataggactgcagtcctcatgtactatggtagtgccatggattagcaacaccacgtatcggcaaaccatagatgatagtttcaccgaaggcggatacatcagcgtcttctaccaaactagaatagtcgtccctctttcgacacccagagagatggacatccttggttttgtgtcagcgtgtaatgacttcagcgtgcgcttgttgcgagataccacacatatagagcaaaaagcgctagcacaggggttaggtcagatgcttgaaagcatgattgacaacacagtccgtgaaacggtgggggcggcaacatctagagacgctctcccaaacactgaagccagtggaccaacacactccaaggaaattccggcactcaccgcagtggaaactggggccacaaatccactagtcccttctgatacagtgcaaaccagacatgttgtacaacataggtcaaggtcagagtctagcatagagtctttcttcgcgcggggtgcatgcgtgaccattatgaccgtggataacccagcttccaccacgaataaggataagctttttgcagtgtggaagatcacttataaagatactgtccagttacggaggaaattggagttcttcacctattctagatttgatatggaacttacctttgtggttactgcaaatttcactgagactaacaatggccatgcattaaatcaagtgtaccaaattatgtacgtaccaccaggcgctccagtgcccgaaaaatgggacgactacacatggcaaacctcatcaaatccatcaatcttttacacctacgggacagctccagcccggatctcggtaccgtatgttggtatttcgaacgcctattcacacttttacgacggtttttccaaagtaccactgaaggaccagtcggcagcactaggtgactccctttatggtgcagcatctctaaatgacttcggtattttggctgttagagtagtcaatgatcacaacccgaccaaggtcacctccaaaatcagagtgtatctaaaacccaaacacatcagagtctggtgcccgcgtccaccgagggcagtggcgtactacggccctggagtggattacaaggatggtacgcttacacccctctccaccaaggagctcaccacatatggattcggacaccaaaacaaagcggtgtacactgcaggttacaaaatttgcaactaccacttggccactcaggatgatttgcaaaacgcagtgaacgtcatgtggagtagagacctcttagtcacagaatcaagagcccagggcaccgattcaatcgcaaggtgcaattgcaacgcaggggtgtactactgcgagtctagaaggaaatactacccagtatccttcgttggcccaacgttccagtacatggaggctaataactattacccagctaggtaccagtcccatatgctcattggccatggattcgcatctccaggggattgtggtggcatactcagatgtcaccacggggtgatagggatcattactgctggtggagaagggttggttgcattttcagacattagagacttgtatgcctacgaagaagaagccatggaacaaggcctcaccaattacatagagtcacttggggccgcatttggaagtggatttactcagcagattagcgacaaaataacagagttgaccaatatggtgaccagtaccatcactgaaaagctacttaagaacttgatcaagatcatatcctcactagttattataactaggaactatgaagacaccacaacagtgctcgctaccctggcccttcttgggtgtgatgcttcaccatggcagtggcttagaaagaaagcatgcgatgttctggagataccttatgtcatcaagcaaggtgacagttggttgaagaagtttactgaagcatgcaacgcagctaagggactggagtgggtgtcaaacaaaatctcaaaattcattgattggctcaaggagaaaattatcccacaagctagagataagttggaatttgtaacaaaacttagacaactagaaatgctggaaaaccaaatctcaactatacaccaatcatgccctagtcaggaacaccaggaaattctattcaataatgtcagatggttatccatccagtctaagaggtttgcccctctttacgcagtggaagccaaaagaatacagaaactcgagcatactattaacaactacatacaatttaagagccaacaccgtatcgaaccagtatgtttgctagtacatggcagccccggaacaggtaaatctgtagcaaccaacctgattgctagagccatagctgaaagagaaaacacgtccacgtactcgctacccccggatccatcacacttcgacggatacaaacaacagggagtggtgattatggacgacctgaatcaaaacccagatggtgcggacatgaagctgttctgtcagatggtatcaacagtggagtttataccacccatggcatccctggaggagaaaggaatcctgtttacttcaaattacgttctagcatccacaaactcaagcagaatttccccccccactgtggcacacagtgacgcgttagccaggcgctttgcgttcgacatggacattcaggtcatgaatgagtattctagagatgggaaattgaacatggccatggctactgaaatgtgtaagaactgtcaccaaccagcaaactttaagagatgctgtcctttagtgtgtggtaaggcaattcaattaatggacaaatcttccagagttagatacagtattgaccagatcactacaatgattatcaatgagagaaacagaagatccaacattggcaattgtatggaggctttgtttcaaggaccactccagtataaagacttgaaaattgacatcaagacgagtccccctcctgaatgtatcaatgacttgctccaagcagttgactcccaggaggtgagagattactgtgagaagaagggttggatagttaacatcaccagccaggttcaaacagaaaggaacatcaacagggcaatgacaattctacaagcggtgacaaccttcgccgcagtggctggagttgtctatgtcatgtataaactgtttgctggacaccagggagcatacactggtttaccaaacaaaaaacccaacgtgcccaccattcggacagcaaaggtacaaggaccagggttcgattacgcagtggctatggctaaaagaaacattgttacagcaactactagcaagggagagttcactatgttaggagtccacgacaacgtggctattttaccaacccacgcttcacctggtgaaagcattgtgatcgatggcaaagaagtggagatcttggatgccaaagcgctcgaagatcaagcaggaaccaatcttgaaatcactataatcactctaaagagaaatgaaaagttcagagacattagaccacatatacctactcaaatcactgagacaaatgatggggtcttgatcgtgaacactagcaagtaccccaatatgtatgttcctgtcggtgctgtgactgaacagggatatctaaatctcggtgggcgccaaactgctcgtactctaatgtacaactttccaaccagagcaggacagtgtggtggagtcatcacatgtactgggaaagtcatcgggatgcatgttggtgggaacggttcacacgggtttgcagcggccctgaagcgatcatacttcactcagagtcaaggtgaaatccagtggatgagaccttcgaaggaagtgggatatccaatcataaatgccccgtccaaaaccaagcttgaacccagtgctttccactatgtgtttgaaggggtgaaggaaccagcagtcctcactaaaaacgatcccaggcttaagacagactttgaggaggcaattttctccaagtacgtgggtaacaaaattactgaagtggatgagtacatgaaagaggcagtagaccactatgctggccagctcatgtcactagacatcaacatagaacaaatgtgcttggaggatgccatgtatggcactgatggtctagaagcacttgatttgtccaccagtgctggctacccttatgtagcaatgggaaagaagaagagagacatcttgaacaaacaaaccagagacactaaggaaatgcaaaaactgctcgacacatatggaatcaacctcccactggtgacttatgtaaaggatgaacttagatccaaaacaaaggttgagcaggggaaatccagattaattgaagcttctagtttgaatgactcagtggcaatgagaatggcttttgggaacctatatgctgcttttcacaaaaacccaggagtgataacaggttcagcagtggggtgcgatccagatttgttttggagcaaaattccggtattgatggaagagaagctgtttgcttttgactacacagggtatgatgcatctctcagccctgcttggttcgaggcactaaagatggtgcttgagaaaatcggattcggagacagagttgactacatcgactacctaaaccactcacaccacctgtacaagaataaaacatactgtgtcaagggcggtatgccatctggctgctcaggcacttcaatttttaactcaatgattaacaacttgattatcaggacactcttactgaaaacctacaagggcatagatttagaccacctaaaaatgattgcctatggtgatgatgtaattgcttcctacccccatgaagttgacgctagtctcctagcccaatcaggaaaagactatggactaactatgactccagctgacaaatcagctacatttgaaacagtcacatgggagaatgtaacattcttgaagagattcttcagggcagacgagaaatacccatttcttattcatccagtaatgccaatgaaggaaattcatgaatcaattagatggactaaagatcctaggaacactcaggatcacgttcgctctctgtgccttttagcttggcacaatggcgaagaagaatataacaaattcctagctaaaatcaggagtgtgccaattggaagagctttattgctcccagagtactcaacattgtaccgccgttggcttgactcattttagtaaccctacctcagtcgaattggattgggtcatactgttgtaggggtaaatttttctttaattcggaggaaaaaaaaaaaaaaaaaaaaaaaaaaaaaaaaaaaaaaaaaaaaaaaaaaaaaaaaaaa

SEQ ID NO: 2 - DNA, 2643 bp, Artificial Sequence
  attenuated poliovirus
atgggagctcaggtgtcatcccaaaaagtaggcgcacacgaaaactcgaatcgggcatacggatctagtacgattaactatactacaatcaattactatagggactccgcctctaacgccgcttcgaaacaggacttttcgcaagaccctagcaagtttaccgaaccgattaaggacgtgttgatcaaaaccgcacctatgcttaactcacctaacatagaggcatgcggatactccgatagggtgttgcaactgacactcggcaatagtacgattacgacacaggaagccgctaatagcgtagtcgcatacggaaggtggcccgaataccttagagactccgaagcgaatccagtcgatcagcctaccgaacctgacgtcgccgcatgtcggttttacacactcgataccgtgtcatggactaaggaatcgcgagggtggtggtggaaactgccagacgcattgcgcgatatggggttgttcggacagaatatgtactatcactatctcggaagatccgggtataccgtacacgtgcaatgtaacgcctctaagtttcaccagggagcgttaggcgtattcgcagtgccagagatgtgtctagccggagactccaatacgactactatgcatacctcataccaaaacgctaacccaggcgaaaagggggggacatttaccggaacattcacacccgataacaatcagacatcccccgctagacggttttgcccagtcgactatctactcggaaacggtacactgttagggaatgccttcgtattcccacaccagataattaacttacggactaacaattgcgcaaccctagtgttgccatacgttaactcactgtcaatcgatagtatggtgaaacataacaattgggggatcgcaatattaccgttagcgccactgaatttcgccagcgaatcgtcacctgagataccgattacccttacaatcgcacctatgtgttgcgagttcaacggattgcgtaatataaccctaccacggttgcaggggttgcccgttatgaataccccagggtctaaccaataccttaccgccgataatttccaatccccttgcgcactgccagagttcgacgtaacccctccaatcgacatacccggcgaggttaagaatatgatggagttagccgaaatcgatactatgataccgttcgatctatccgctacgaaaaagaatactatggagatgtatcgcgtgagattgtccgataagccacataccgacgatccgatactgtgtctgtcactgtcacccgccagcgatcctaggttgtcccatacaatgttaggcgagatactgaattactatacccattgggccggtagtctgaaattcacatttctgttttgcggatctatgatggcgaccggaaagctgttagtgtcatacgctccacccggagccgatccacctaaaaaacgcaaggaagcgatgctcggtacacacgtgatatgggatatcggactgcaatcgtcatgtactatggtcgtgccatggatatcgaatacgacttatagacagacaatcgacgatagctttaccgagggggggtatattagcgtattctatcagacacgtatcgtagtgccactgtcaacccctagagagatggacatactcggattcgtatccgcatgtaacgactttagcgtgagactgttacgcgatactacccatatcgaacagaaagcgctagcacaggggttagggcaaatgctagagtcaatgatcgacaatactgtacgcgaaaccgtaggcgcagcgaccagtagggacgcactaccgaataccgaagcgagcggacctacccactctaaagagataccggcactaaccgccgtcgaaaccggagcgactaacccactagtcccatccgataccgtgcaaaccagacacgtagtgcaacatcggtctagatccgagtcatcaatcgaatcctttttcgctagaggcgcatgcgttacgattatgaccgtcgataacccagcgtcaacgactaacaaagacaaattgttcgccgtatggaaaattacctataaggataccgtgcaattgcgacgtaaactagagttctttacatactctagattcgatatggagcttacattcgtagtgaccgctaactttaccgagactaataacggtcacgcccttaatcaggtgtatcagattatgtacgtaccgccaggcgcaccagtgcccgaaaaatgggacgactatacatggcagactagctctaatccgtcaattttctatacatacggtaccgcaccagctaggattagcgtgccatacgtcggtatatcgaacgcttactcacacttttacgacggattctctaaagtgccacttaaggaccaatccgccgcactaggcgatagcctatacggagccgctagtcttaacgatttcggtatactcgccgttagggtcgtgaacgaccataaccctactaaggtcacctctaagattagggtgtatcttaagcctaagcatattagggtgtggtgtcctagaccgcctagagccgttgcgtattacggaccaggcgtcgactataaggacggtacattgaccccactgtcaacgaaagaccttacgacgtac

SEQ ID NO: 3 - DNA, 7579 bp, Artificial Sequence
  PV-1(mono-cre-X) attenuated poliovirus
ttaaaacagctctggggttgtacccaccccagaggcccacgtggcggctagtactccggtattgcggtacccttgtacgcctgttttatactcccttcccgctagcactattaacaactacatacagttcaagagcaaacaccgtattgaaccagtatgtttgctagtagctagcttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatgggagctcaggtgtcatcccaaaaagtaggcgcacacgaaaactcgaatcgggcatacggatctagtacgattaactatactacaatcaattactatagggactccgcctctaacgccgcttcgaaacaggacttttcgcaagaccctagcaagtttaccgaaccgattaaggacgtgttgatcaaaaccgcacctatgcttaactcacctaacatagaggcatgcggatactccgatagggtgttgcaactgacactcggcaatagtacgattacgacacaggaagccgctaatagcgtagtcgcatacggaaggtggcccgaataccttagagactccgaagcgaatccagtcgatcagcctaccgaacctgacgtcgccgcatgtcggttttacacactcgataccgtgtcatggactaaggaatcgcgagggtggtggtggaaactgccagacgcattgcgcgatatggggttgttcggacagaatatgtactatcactatctcggaagatccgggtataccgtacacgtgcaatgtaacgcctctaagtttcaccagggagcgttaggcgtattcgcagtgccagagatgtgtctagccggagactccaatacgactactatgcatacctcataccaaaacgctaacccaggcgaaaagggggggacatttaccggaacattcacacccgataacaatcagacatcccccgctagacggttttgcccagtcgactatctactcggaaacggtacactgttagggaatgcctttgtgttcccgcaccagataataaacctacggaccaacaactgtgctacactggtactcccttacgtgaactccctctcgatagatagtatggtaaagcacaataattggggaattgcaatattaccattggccccattaaattttgctagtgagtcctccccagagattccaatcaccttgaccatagcccctatgtgctgtgagttcaatggattaagaaacatcaccctgccacgcttacagggcctgccggtcatgaacacccctggtagcaatcaatatcttactgcagacaacttccagtcaccgtgtgcgctgcctgaatttgatgtgaccccacctattgacatacccggtgatgatggaattggcagaaatcgacaccatgattccctttgacttaagtgccacaaaaaagaacaccatggaaatgtatagggttcggttaagtgacaaaccacatacagacgatcccatactctgcctgtcactctctccagcctcagatcctaggttgtcacatactatgcttggagaaatcctaaattactacacacactgggcaggatccctgaagttcacgtttctgttctgtggatccatgatggcaactggcaaactgttggtgtcatacgcgcctcctggagccgacccaccaaagaagcgtaaggaggcgatgttgggaacacatgtgatctgggacataggactgcagtcctcatgtactatggtagtgccatggattagcaacaccacgtatcggcaaaccatagatgatagtttcaccgaaggcggatacatcagcgtcttctaccaaactagaatagtcgtccctctttcgacacccagagagatggacatccttggttttgtgtcagcgtgtaatgacttcagcgtgcgcttgttgcgagataccacacatatagagcaaaaagcgctagcacaggggttaggtcagatgcttgaaagcatgattgacaacacagtccgtgaaacggtgggggcggcaacatctagagacgctctcccaaacactgaagccagtggaccaacacactccaaggaaattccggcactcaccgcagtggaaactggggccacaaatccactagtcccttctgatacagtgcaaaccagacatgttgtacaacataggtcaaggtcagagtctagcatagagtctttcttcgcgcggggtgcatgcgtgaccattatgaccgtggataacccagcttccaccacgaataaggataagctttttgcagtgtggaagatcacttataaagatactgtccagttacggaggaaattggagttcttcacctattctagatttgatatggaacttacctttgtggttactgcaaatttcactgagactaacaatggccatgcattaaatcaagtgtaccaaattatgtacgtaccaccaggcgctccagtgcccgaaaaatgggacgactacacatggcaaacctcatcaaatccatcaatcttttacacctacgggacagctccagcccggatctcggtaccgtatgttggtatttcgaacgcctattcacacttttacgacggtttttccaaagtaccactgaaggaccagtcggcagcactaggtgactccctttatggtgcagcatctctaaatgacttcggtattttggctgttagagtagtcaatgatcacaacccgaccaaggtcacctccaaaatcagagtgtatctaaaacccaaacacatcagagtctggtgcccgcgtccaccgagggcagtggcgtactacggccctggagtggattacaaggatggtacgcttacacccctctccaccaaggatctgaccacatatggattcggacaccaaaacaaagcggtgtacactgcaggttacaaaatttgcaactaccacttggccactcaggatgatttgcaaaacgcagtgaacgtcatgtggagtagagacctcttagtcacagaatcaagagcccagggcaccgattcaatcgcaaggtgcaattgcaacgcaggggtgtactactgcgagtctagaaggaaatactacccagtatccttcgttggcccaacgttccagtacatggaggctaataactattacccagctaggtaccagtcccatatgctcattggccatggattcgcatctccaggggattgtggtggcatactcagatgtcaccacggggtgatagggatcattactgctggtggagaagggttggttgcattttcagacattagagacttgtatgcctacgaagaagaagccatggaacaaggcctcaccaattacatagagtcacttggggccgcatttggaagtggatttactcagcagattagcgacaaaataacagagttgaccaatatggtgaccagtaccatcactgaaaagctacttaagaacttgatcaagatcatatcctcactagttattataactaggaactatgaagacaccacaacagtgctcgctaccctggcccttcttgggtgtgatgcttcaccatggcagtggcttagaaagaaagcatgcgatgttctggagataccttatgtcatcaagcaaggtgacagttggttgaagaagtttactgaagcatgcaacgcagctaagggcctggagtgggtgtcaaacaaaatctcaaaattcattgattggctcaaggagaaaattatcccacaagctagagataagttggaatttgtaacaaaacttagacaactagaaatgctggaaaaccaaatctcaactatacaccaatcatgccctagtcaggaacaccaggaaattctattcaataatgtcagatggttatccatccagtctaagaggtttgcccctctttacgcagtggaagccaaaagaatacagaaactcgagcatactattaacaactacatacaatttaagagccaacaccgtatcgaaccagtatgtttgctagtacatggcagccccggaacaggtaaatctgtagcaaccaacctgattgctagagccatagctgaaagagaaaacacgtccacgtactcgctacccccggatccatcacacttcgacggatacaaacaacagggagtggtgattatggacgacctgaatcaaaacccagatggtgcggacatgaagctgttctgtcagatggtatcaacagtggagtttataccacccatggcatccctggaggagaaaggaatcctgtttacttcaaattacgttctagcatccacaaactcaagcagaatttccccccccactgtggcacacagtgacgcgttagccaggcgctttgcgttcgacatggacattcaggtcatgaatgagtattctagagatgggaaattgaacatggccatggctactgaaatgtgtaagaactgtcaccaaccagcaaactttaagagatgctgtcctttagtgtgtggtaaggcaattcaattaatggacaaatcttccagagttagatacagtattgaccagatcactacaatgattatcaatgagagaaacagaagatccaacattggcaattgtatggaggctttgtttcaaggaccactccagtataaagacttgaaaattgacatcaagacgagtccccctcctgaatgtatcaatgacttgctccaagcagttgactcccaggaggtgagagattactgtgagaagaagggttggatagttaacatcaccagccaggttcaaacagaaaggaacatcaacagggcaatgacaattctacaagcggtgacaaccttcgccgcagtggctggagttgtctatgtcatgtataaactgtttgctggacaccagggagcatacactggtttaccaaacaaaaaacccaacgtgcccaccattcggacagcaaaggtacaaggaccagggttcgattacgcagtggctatggctaaaagaaacattgttacagcaactactagcaagggagagttcactatgttaggagtccacgacaacgtggctattttaccaacccacgcttcacctggtgaaagcattgtgatcgatggcaaagaagtggagatcttggatgccaaagcgctcgaagatcaagcaggaaccaatcttgaaatcactataatcactctaaagagaaatgaaaagttcagagacattagaccacatatacctactcaaatcactgagacaaatgatggagtcttgatcgtgaacactagcaagtaccccaatatgtatgttcctgtcggtgctgtgactgaacagggatatctaaatctcggtgggcgccaaactgctcgtactctaatgtacaactttccaaccagagcaggacagtgtggtggagtcatcacatgtactgggaaagtcatcgggatgcatgttggtgggaacggttcacacgggtttgcagcggccctgaagcgatcatacttcactcagagtcaaggtgaaatccagtggatgagaccttcgaaggaagtgggatatccaatcataaatgccccgtccaaaaccaagcttgaacccagtgctttccactatgtgtttgaaggggtgaaggaaccagcagtcctcactaaaaacgatcccaggcttaagacagactttgaggaggcaattttctccaagtacgtgggtaacaaaattactgaagtggatgagtacatgaaagaggcagtagaccactatgctggccagctcatgtcactagacatcaacacagaacaaatgtgcttggaggatgccatgtatggcactgatggtctagaagcacttgatttgtccaccagtgctggctacccttatgtagcaatgggaaagaagaagagagacatcttgaacaaacaaaccagagacactaaggaaatgcaaaaactgctcgacacatatggaatcaacctcccactggtgacttatgtaaaggatgaacttagatccaaaacaaaggttgagcaggggaaatccagattaattgaagcttctagtttgaatgactcagtggcaatgagaatggcttttgggaacctatatgctgcttttcacaaaaacccaggagtgataacaggttcagcagtggggtgcgatccagatttgttttggagcaaaattccggtattgatggaagagaagctgtttgcttttgactacacagggtatgatgcatctctcagccctgcttggttcgaggcactaaagatggtgcttgagaaaatcggattcggagacagagttgactacatcgactacctaaaccactcacaccacctgtacaagaataaaacatactgtgtcaagggcggtatgccatctggctgctcaggcacttcaatttttaactcaatgattaacaacttgattatcaggacactcttactgaaaacctacaagggcatagatttagaccacctaaaaatgattgcctatggtgatgatgtaattgcttcctacccccatgaagttgacgctagtctcctagcccaatcaggaaaagactatggactaactatgactccagctgacaaatcagctacatttgaaacagtcacatgggagaatgtaacattcttgaagagattcttcagggcagacgagaaatacccatttcttattcatccagtaatgccaatgaaggaaattcatgaatcaattagatggactaaagatcctaggaacactcaggatcacgttcgctctctgtgccttttagcttggcacaatggcgaagaagaatataacaaattcctagctaaaatcaggagtgtgccaattggaagagctttattgctcccagagtactcaacattgtaccgccgttggcttgactcattttagtaaccctacctcagtcgaattggattgggtcatactgttgtaggggtaaatttttctttaattcggagaaaaaaaaaaaaaaaaaaaaaaaaaaaaaaaaaaaaaaaaaaaaaaaaaaaaaaaaaaaaaaaaaaaaaaaaaaaaaaaa